SEQUENCE LISTING (96 sequences)

SEQ ID NO: 1 - protein, 80 aa, synthetic construct
GSPSGESSAPGSSSAESPGPGSESPSGSAPGESSPSGSAPGSPSGESSAPGSPESGSASPGSSSAESPGPGSPSGESSAP

SEQ ID NO: 2 - protein, 100 aa, synthetic construct
GSPSGESSAPGSSSAESPGPGSESPSGSAPGESSPSGSAPGSPSGESSAPGSPESGSASPGSESPSGSAPGSSSAESPGPGSPSGESSAPGESSPSGSAP

SEQ ID NO: 3 - protein, 96 aa, synthetic construct
GPEGPSPEGPESEPGGSPESPEGPSPEGPESPSGPGSGGESPGEPSGPEGPESPGSPSGPGGSGPEPSGGESSGEGPSSPEGPGSPGGSPEGPSGP

SEQ ID NO: 4 - protein, 163 aa, synthetic construct
GSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 5 - protein, 240 aa, synthetic construct
GSPSGESSAPGSSSAESPGPGSESPSGSAPGESSPSGSAPGSPSGESSAPGSPESGSASPGSSSAESPGPGSPSGESSAPGSPSGESSAPGSSSAESPGPGSESPSGSAPGESSPSGSAPGSPSGESSAPGSPESGSASPGSSSAESPGPGSPSGESSAPGSPSGESSAPGSSSAESPGPGSESPSGSAPGESSPSGSAPGSPSGESSAPGSPESGSASPGSSSAESPGPGSPSGESSAP

SEQ ID NO: 6 - protein, 62 aa, synthetic construct
GSPSGESSAPGSSSAESPGPGSESPSGSAPGESSPSGSAPGSPSGESSAPGSPESGSASPGS

SEQ ID NO: 7 - protein, 96 aa, synthetic construct
APSAPSPSAPESSPAASPESPSAPSPEAPSSPSAPASAPESPASPSAPSAPESPASPSAPAPSAPSPSAPESSPEAPSSPSAPASPAASPESPSAP

SEQ ID NO: 8 - protein, 10 aa, synthetic construct
MVPSALTASG

SEQ ID NO: 9 - protein, 10 aa, synthetic construct
MVLSAVTASG

SEQ ID NO: 10 - protein, 10 aa, synthetic construct
MVVLALTASG

SEQ ID NO: 11 - protein, 10 aa, synthetic construct
MVLSAPTASG

SEQ ID NO: 12 - protein, 10 aa, synthetic construct
MVPSAVTASG

SEQ ID NO: 13 - protein, 10 aa, synthetic construct
MVPSAYTASG

SEQ ID NO: 14 - protein, 10 aa, synthetic construct
MVASALTASG

SEQ ID NO: 15 - protein, 10 aa, synthetic construct
MVVSALTASG

SEQ ID NO: 16 - protein, 10 aa, synthetic construct
MVPSALTVSG

SEQ ID NO: 17 - skipped

SEQ ID NO: 18 - protein, 5 aa, synthetic construct
GGGGS

SEQ ID NO: 19 - protein, 10 aa, synthetic construct
GGGGSGGGGS

SEQ ID NO: 20 - protein, 15 aa, synthetic construct
GGGGSGGGGSGGGGS

SEQ ID NO: 21 - protein, 25 aa, synthetic construct
GGGGSGGGGSGGGGSGGGGSGGGGS

SEQ ID NO: 22 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 24 - protein, 65 aa, synthetic construct
ITCPPPMSVEHADIWVKSYSLYSRERYICNSGFKRKAGTSSLTECVLNKATNVAHWTTPSLKCIR

SEQ ID NO: 25 - protein, 64 aa, synthetic construct
TCPPPMSVEHADIWVKSYSLYSRERYICNSGFKRKAGTSSLTECVLNKATNVAHWTTPSLKCIR

SEQ ID NO: 26 - protein, 77 aa, synthetic construct
ITCPPPMSVEHADIWVKSYSLYSRERYICNSGFKRKAGTSSLTECVLNKATNVAHWTTPSLKCIRDPALVHQRPAPP

SEQ ID NO: 27 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 348 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 32 - protein, 321 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSGGGGSGGGGSGGGGSGGGGSDIITCPPPMSVEHADIWVKSYSLYSRERYICNSGFKRKAGTSSLTECVLNKATNVAHWTTPSLKCIR

SEQ ID NO: 33 - protein, 461 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAP

SEQ ID NO: 34 - protein, 461 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVLSAVTASGGGGGSGGGGSRGSPSGESSAPGSSSAESPGPGSESPSGSAPGESSPSGSAPGSPSGESSAPGSPESGSASPGSSSAESPGPGSPSGESSAP

SEQ ID NO: 35 - protein, 441 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSRGSPSGESSAPGSSSAESPGPGSESPSGSAPGESSPSGSAPGSPSGESSAPGSPESGSASPGSSSAESPGPGSPSGESSAP

SEQ ID NO: 36 - protein, 461 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVVLALTASGGGGGSGGGGSRGSPSGESSAPGSSSAESPGPGSESPSGSAPGESSPSGSAPGSPSGESSAPGSPESGSASPGSSSAESPGPGSPSGESSAP

SEQ ID NO: 37 - protein, 477 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGPEGPSPEGPESEPGGSPESPEGPSPEGPESPSGPGSGGESPGEPSGPEGPESPGSPSGPGGSGPEPSGGESSGEGPSSPEGPGSPGGSPEGPSGP

SEQ ID NO: 38 - protein, 481 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSESPSGSAPGSSSAESPGPGSPSGESSAPGESSPSGSAP

SEQ ID NO: 39 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 40 - protein, 294 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGTCPPPMSVEHADIWVKSYSLYSRERYICNSGFKRKAGTSSLTECVLNKATNVAHWTTPSLKCIR

SEQ ID NO: 41 - protein, 311 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSGGGGSGGGGSDIITCPPPMSVEHADIWVKSYSLYSRERYICNSGFKRKAGTSSLTECVLNKATNVAHWTTPSLKCIR

SEQ ID NO: 42 - protein, 348 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 43 - protein, 311 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSGGGGSGGGGSDIITCPPPMSVEHADIWVKSYSLYSRERYICNSGFKRKAGTSSLTECVLNKATNVAHWTTPSLKCIR

SEQ ID NO: 44 - protein, 348 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 45 - protein, 301 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSDIITCPPPMSVEHADIWVKSYSLYSRERYICNSGFKRKAGTSSLTECVLNKATNVAHWTTPSLKCIR

SEQ ID NO: 46 - protein, 544 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 47 - protein, 621 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSPSGESSAPGSSSAESPGPGSESPSGSAPGESSPSGSAPGSPSGESSAPGSPESGSASPGSSSAESPGPGSPSGESSAPGSPSGESSAPGSSSAESPGPGSESPSGSAPGESSPSGSAPGSPSGESSAPGSPESGSASPGSSSAESPGPGSPSGESSAP

SEQ ID NO: 48 - protein, 446 aa, synthetic construct
EEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTSRGSPSGESSAPGSSSAESPGPGSESPSGSAPGESSPSGSAPGSPSGESSAPGSPESGSASPGSSSAESPGPGSPSGESSAP

SEQ ID NO: 49 - protein, 10 aa, synthetic construct
  VARIANT 3: note = X is P, L, V or A
  VARIANT 4: note = X is S or L
  VARIANT 6: note = X is L, V, P or Y
  VARIANT 8: note = X is A or V
MVXXAXTXSG

SEQ ID NO: 50 - protein, 5 aa, synthetic construct
DDYID

SEQ ID NO: 51 - protein, 17 aa, synthetic construct
EIYPGSGNTYYNEKFKG

SEQ ID NO: 52 - protein, 11 aa, synthetic construct
SQVYGKIAMDH

SEQ ID NO: 53 - protein, 15 aa, synthetic construct
RASESVDNSGNSFMH

SEQ ID NO: 54 - protein, 7 aa, synthetic construct
RASNLES

SEQ ID NO: 55 - protein, 9 aa, synthetic construct
QQSKEDPYT

SEQ ID NO: 56 - protein, 120 aa, synthetic construct
QVQLVQSGAEVKKPGASVKVSCKASGYTFTDDYIDWVRQATGQGLEWIGEIYPGSGNTYYNEKFKGRVTMTRDKSISTAYMELSSLRSEDTAVYYCARSQVYGKIAMDHWGQGTLVTVSS

SEQ ID NO: 57 - protein, 111 aa, synthetic construct
DIVMTQSPDSLAVSLGERATINCRASESVDNSGNSFMHWYQQKPGQPPKLLIYRASNLESGVPDRFSGSGSRTDFTLTISSLQAEDVAVYYCQQSKEDPYTFGQGTKVEIK

SEQ ID NO: 58 - protein, 560 aa, synthetic construct
GSPSGESSAPGSSSAESPGPGSESPSGSAPGESSPSGSAPGSPSGESSAPGSPESGSASPGSSSAESPGPGSPSGESSAPGGGGSGGGGSMVPSALTASGGGGGSGGGGSQVQLVQSGAEVKKPGASVKVSCKASGYTFTDDYIDWVRQATGQGLEWIGEIYPGSGNTYYNEKFKGRVTMTRDKSISTAYMELSSLRSEDTAVYYCARSQVYGKIAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 218 aa, synthetic construct
DIVMTQSPDSLAVSLGERATINCRASESVDNSGNSFMHWYQQKPGQPPKLLIYRASNLESGVPDRFSGSGSRTDFTLTISSLQAEDVAVYYCQQSKEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 450 aa, synthetic construct
QVQLVQSGAEVKKPGASVKVSCKASGYTFTDDYIDWVRQATGQGLEWIGEIYPGSGNTYYNEKFKGRVTMTRDKSISTAYMELSSLRSEDTAVYYCARSQVYGKIAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 328 aa, synthetic construct
GSPSGESSAPGSSSAESPGPGSESPSGSAPGESSPSGSAPGSPSGESSAPGSPESGSASPGSSSAESPGPGSPSGESSAPGGGGSGGGGSMVPSALTASGGGGGSGGGGSDIVMTQSPDSLAVSLGERATINCRASESVDNSGNSFMHWYQQKPGQPPKLLIYRASNLESGVPDRFSGSGSRTDFTLTISSLQAEDVAVYYCQQSKEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 321 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSGGGGSGGGGSGGGGSGGGGSDIITCPPPMSVEHADIWVKSYSLYSRERYICNSGFKRKAGTSSLTECVLNKATNVAHWTTPSLKCIR

SEQ ID NO: 63 - protein, 461 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAP

SEQ ID NO: 64 - protein, 294 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGSGTCPPPMSVEHADIWVKSYSLYSRERYICNSGFKRKAGTSSLTECVLNKATNVAHWTTPSLKCIR

SEQ ID NO: 65 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 66 - protein, 301 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSDIITCPPPMSVEHADIWVKSYSLYSRERYICNSGFKRKAGTSSLTECVLNKATNVAHWTTPSLKCIR

SEQ ID NO: 67 - protein, 114 aa, synthetic construct
NWVNVISDLKKIEDLIQSMHIDATLYTESDVHPSCKVTAMKCFLDELQVISLESGDASIHDTVENLIILANNSLSSNGNVTESGCKECEELEEKNIKEFLQSFVHIVQMFINTS

SEQ ID NO: 68 - protein, 114 aa, synthetic construct
NWVNVISDLKKIEDLIQSMHIDATLYTESDVHPSCKVTAMKCFLEELQVISLESGDASIHDTVENLIILANNSLSSNGNVTESGCKECEELEEKNIKEFLQSFVHIVQMFINTS

SEQ ID NO: 69 - protein, 114 aa, synthetic construct
NWVNVISDLKKIEDLIQSMHIDATLYTESDVHPSCKVTAMKCFLLELKVISLESGDASIHDTVENLIILANNSLSSNGNVTESGCKECEELEEKNIKEFLQSFVHIVQMFINTS

SEQ ID NO: 70 - protein, 114 aa, synthetic construct
NWVNVISDLKKIEDLIQSMHIDATLYTESDVHPSCKVTAMKCFLLELQVIDLESGDASIHDTVENLIILANNSLSSNGNVTESGCKECEELEEKNIKEFLQSFVHIVQMFINTS

SEQ ID NO: 71 - protein, 114 aa, synthetic construct
NWVNVISDLKKIEDLIQSMHIDATLYTESDVHPSCKVTAMKCFLLELQVISDESGDASIHDTVENLIILANNSLSSNGNVTESGCKECEELEEKNIKEFLQSFVHIVQMFINTS

SEQ ID NO: 72 - protein, 114 aa, synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 73 - protein, 114 aa, synthetic construct
NWVNVISDLKKIEDLIQSMHIDATLYTESDVHPSCKVTAMKCFLLELQVIQLESGDASIHDTVKNLIILANNSLSSNGNVTESGCKECEELEEKNIKEFLQSFVHIVQMFINTS

SEQ ID NO: 74 - protein, 114 aa, synthetic construct
NWVNVISDLKKIEDLIQSMHIDATLYTESDVHPSCKVTAMKCFLLELQVIQLESGDASIHDTVENLDILANNSLSSNGNVTESGCKECEELEEKNIKEFLQSFVHIVQMFINTS

SEQ ID NO: 75 - protein, 114 aa, synthetic construct
NWVNVISDLKKIEDLIQSMHIDATLYTESDVHPSCKVTAMKCFLLELQVIQLESGDASIHDTVENLEILANNSLSSNGNVTESGCKECEELEEKNIKEFLQSFVHIVQMFINTS

SEQ ID NO: 76 - protein, 114 aa, synthetic construct
NWVNVISDLKKIEDLIQSMHIDATLYTESDVHPSCKVTAMKCFLLELQVIQLESGDASIHDTVENLIDLANNSLSSNGNVTESGCKECEELEEKNIKEFLQSFVHIVQMFINTS

SEQ ID NO: 77 - protein, 348 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLDELQVISLESGDASIHDTVENLIILANNSLSSNGNVTESGCKECEELEEKNIKEFLQSFVHIVQMFINTS

SEQ ID NO: 78 - protein, 348 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLEELQVISLESGDASIHDTVENLIILANNSLSSNGNVTESGCKECEELEEKNIKEFLQSFVHIVQMFINTS

SEQ ID NO: 79 - protein, 348 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELKVISLESGDASIHDTVENLIILANNSLSSNGNVTESGCKECEELEEKNIKEFLQSFVHIVQMFINTS

SEQ ID NO: 80 - protein, 348 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVIDLESGDASIHDTVENLIILANNSLSSNGNVTESGCKECEELEEKNIKEFLQSFVHIVQMFINTS

SEQ ID NO: 81 - protein, 348 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISDESGDASIHDTVENLIILANNSLSSNGNVTESGCKECEELEEKNIKEFLQSFVHIVQMFINTS

SEQ ID NO: 82 - protein, 348 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVKNLIILANNSLSSNGNVTESGCKECEELEEKNIKEFLQSFVHIVQMFINTS

SEQ ID NO: 83 - protein, 348 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLDILANNSLSSNGNVTESGCKECEELEEKNIKEFLQSFVHIVQMFINTS

SEQ ID NO: 84 - protein, 348 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLEILANNSLSSNGNVTESGCKECEELEEKNIKEFLQSFVHIVQMFINTS

SEQ ID NO: 85 - protein, 348 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIDLANNSLSSNGNVTESGCKECEELEEKNIKEFLQSFVHIVQMFINTS

SEQ ID NO: 86 - protein, 348 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 87 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLDELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 88 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLEELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 89 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELKVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 90 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVIDLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 91 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISDESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 92 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVKNLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 93 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLDILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 94 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLEILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 95 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIDLANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 96 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

(19) United States
(12) Patent Application Publication
(10) Pub. No.: US 2026/0035426 A1
(43) Pub. Date: Feb. 5, 2026

(54) IL-15 PRODRUG AND USES THEREOF

(71) Applicant: STAIDSON BIOPHARMA INC., Richmond, CA (US)

(72) Inventors: Yong YIN, Richmond, CA (US); Wenwu ZHAI, Richmond, CA (US); Zuoan YI, Mountain View, CA (US)

(73) Assignee: STAIDSON BIOPHARMA INC., Richmond, CA (US)

(21) Appl. No.: 18/997,030

(22) PCT Filed: Aug. 4, 2023
(86) PCT No.: PCT/US2023/071645

§ 371 Date: Jan. 18, 2025

Related U.S. Application Data
(60) Provisional application No. 63/370,605, filed on Aug. 5, 2022.
(60) Provisional application No. 63/370,606, filed on Aug. 5, 2022.
(60) Provisional application No. 63/370,607, filed on Aug. 5, 2022.
(60) Provisional application No. 63/477,993, filed on Dec. 30, 2022.

(51) Int. Cl. C07K 14/54; A61K 38/00; A61P 35/00; A61P 37/04; C07K 14/715

(52) U.S. Cl. CPC C07K 14/5443; A61P 35/00; A61P 37/04; C07K 14/7155; A61K 38/00; C07K 2319/30; C07K 2319/50; C07K 2319/70

(57) ABSTRACT

The present application provides IL-15 prodrugs comprising the masking polypeptide (MP) and the cleavable moiety (CM). The isolated nucleic acid molecules encoding the IL-15 prodrugs; vectors comprising the nucleic acid molecules; host cells containing the nucleic acid molecules or vectors; pharmaceutical compositions containing the IL-15 prodrugs, the isolated nucleic acids molecules, the vectors, or the host cells. And methods of producing and using the IL-15 prodrugs or pharmaceutical compositions.

### CROSS REFERENCE TO RELATED APPLICATIONS

This application claims the benefit of U.S. Provisional Application No. 63/370,605, filed on Aug. 5, 2022; 63/370,606, filed on Aug. 5, 2022; 63/370,607, filed on Aug. 5, 2022; and 63/477,993, filed on Dec. 30, 2022, the contents of each of which are incorporated herein by reference in their entirety.

### SUBMISSION OF SEQUENCE LISTING

The contents of the electronic sequence listing (IL-15 prodrug and uses thereof SEQ-230728.xml; Size: 106 KB; and Date of Creation: Jul. 28, 2023) is herein incorporated by reference in its entirety.

### FIELD

The present invention relates to the IL-15 prodrugs and methods of making, and using thereof. This invention also relates to cleavage products of said activatable IL-15 prodrugs and methods of using thereof.

### BACKGROUND

Cytokines are potent immune agonists, which lead to them being considered as promising therapeutic agents for oncology. For example, the antitumoral activity of interleukin-15 (IL-15) is currently under investigation and have already been used therapeutically in human. However, cytokines probed to have a very narrow therapeutic window and a short serum half-life. Consequently, therapeutic administration of cytokines produced undesirable systemic effects and toxicities. These were aggravated by the need to administer large quantities of cytokines to achieve the desired levels of cytokines at the intended site of cytokines action (e.g., a tumor).

Antibodies have been viewed as ideal candidates for use in therapy in the fields of cancer, autoimmunity, and chronic inflammatory disorders, but sometimes the antibody therapy is limited by their cross-reactivity to healthy tissue. Several approaches have been described for overcoming these “off-target” effects by engineering antibodies to improve tumor targeting, for example, by generating masked antibodies that are selectively activated in the tumor microenvironment (see, e.g., WO2003/068934, WO2004/009638, WO 2009/025846, WO2101/081173 and WO2014103973).

The present application refers to conditionally activatable prodrugs (e.g., cytokine prodrug or antibody prodrug) that have a cleavable moiety linked to a masking polypeptide (MP) for use in the treatment of cancer or other diseases. Masking polypeptides (MP) can act via steric hindrance to the biologically active moiety. The cleavable moiety can be designed to be cleaved by proteases that are specific to certain tissues or pathologies, thus enabling the prodrugs to be preferentially activated in desired locations (e.g., a tumor) to overcome the dosing amounts limitation of the cytokines or the “off-target” effects of the antibody.

### BRIEF SUMMARY

The present application provides an IL-15 prodrug, wherein the IL-15 prodrug comprises: (i) one or more IL-15 cytokine (I), (ii) one or more cleavable moiety (CM), and (iii) one or more masking polypeptide (MP).

In some embodiments, the IL-15 prodrug provided herein, wherein the masking polypeptide (MP) attenuates the activity of IL-15 cytokine (I), and the cleavable moiety (CM) is susceptible to cleave at or near a tumor or a target cell.

In some embodiments, the IL-15 prodrug provided herein, wherein the IL-15 cytokine (I) and the masking polypeptide (MP) are linked through the cleavable moiety (CM).

In some embodiments, the IL-15 prodrug provided herein, wherein the IL-15 prodrug further comprises an IL-15Rα or a functional fragment thereof (S), and wherein the IL-15Rα or a functional fragment thereof is selected from an extracellular domain of IL-15Rα or a sushi domain or functional analogs.

In some embodiments, the IL-15 prodrug provided herein, wherein the IL-15Rα or a functional fragment thereof (S) and masking polypeptide (MP) are linked through the cleavable moiety (CM).

In some embodiments, the IL-15 prodrug provided herein, wherein the IL-15 prodrug further comprises one or more half-life extension moiety (C).

In some embodiments, the IL-15 prodrug provided herein, wherein the IL-15 cytokine (I) is linked to the half-life extension moiety (C).

In some embodiments, the IL-15 prodrug provided herein, wherein the IL-15Rα or a functional fragment thereof (S) is linked to the half-life extension moiety (C).

In some embodiments, the IL-15 prodrug provided herein, wherein the masking polypeptide (MP) is linked to the half-life extension moiety (C) through the cleavable moiety (CM).

In some embodiments, the IL-15 prodrug provided herein, wherein the IL-15 cytokine (I) is linked to the half-life extension moiety (C), the IL-15Rα or a functional fragment thereof (S) is linked to the IL-15 cytokine (I), and the masking polypeptide (MP) is linked to the IL-15Rα or a functional fragment thereof (S) through the cleavable moiety (CM).

In some embodiments, the IL-15 prodrug provided herein, wherein the IL-15 cytokine (I) is linked to the half-life extension moiety (C), the IL-15Rα or a functional fragment thereof (S) is linked to the half-life extension moiety (C), and the masking polypeptide (MP) is linked to the IL-15 cytokine (I) through the cleavable moiety (CM).

In some embodiments, the IL-15 prodrug provided herein, wherein the IL-15Rα or a functional fragment thereof (S) is linked to the half-life extension moiety (C), the IL-15 cytokine (I) is linked to the IL-15Rα or a functional fragment thereof (S), and the masking polypeptide (MP) is linked to the IL-15 cytokine (I) through the cleavable moiety (CM).

In some embodiments, the IL-15 prodrug provided herein, wherein the IL-15 cytokine (I) is linked to the half-life extension moiety (C), the IL-15Rα or a functional fragment thereof (S) is linked to the half-life extension moiety (C), and the masking polypeptide (MP) is linked to the IL-15Rα or a functional fragment thereof (S) through the cleavable moiety (CM).

In some embodiments, the IL-15 prodrug provided herein, wherein the masking polypeptide (MP) is linked to the half-life extension moiety (C) through the cleavable moiety (CM), the IL-15Rα or a functional fragment thereof (S) is linked to the half-life extension moiety (C), and the IL-15 cytokine (I) is linked to the IL-15Rα or a functional fragment thereof (S).

In some embodiments, the IL-15 prodrug provided herein, wherein the masking polypeptide (MP) is linked to the half-life extension moiety (C) through the cleavable moiety (CM), the IL-15 cytokine (I) is linked to the half-life extension moiety (C), and the IL-15Rα or a functional fragment thereof (S) is linked to the IL-15 cytokine (I).

In some embodiments, the IL-15 prodrug provided herein is a monomer or a dimer.

In some embodiments, the IL-15 prodrug provided herein comprises two monomers, wherein one monomer comprises the first half-life extension moiety (C), the IL-15Rα or a functional fragment thereof (S), the IL-15 cytokine (I); and the other monomer comprises the second half-life extension moiety (C), the masking polypeptide (MP) and the cleavable moiety (CM), wherein the masking polypeptide (MP) is linked to the second half-life extension moiety (C) through the cleavable moiety (CM).

In some embodiments, the IL-15 prodrug provided herein comprises two monomers, wherein one monomer comprises the first half-life extension moiety (C), the IL-15Rα or a functional fragment thereof (S), the masking polypeptide (MP) and the cleavable moiety (CM); and the other monomer comprises the second half-life extension moiety (C) and the IL-15 cytokine (I).

In some embodiments, the IL-15 prodrug provided herein comprises two monomers, wherein one monomer comprises the first half-life extension moiety (C), the IL-15 cytokine (I), the masking polypeptide (MP) and a cleavable moiety (CM); and the other monomer comprises the second half-life extension moiety (C) and the IL-15Rα or a functional fragment thereof (S).

In some embodiments, the IL-15 prodrug provided herein comprises two monomers, wherein one monomer and the other monomer each comprises the half-life extension moiety (C), the IL-15 cytokine (I), the IL-15Rα or a functional fragment thereof (S), the cleavable moiety (CM) and the masking polypeptide (MP).

In some embodiments, the IL-15 prodrug provided herein comprises two monomers, wherein in one monomer: both the IL-15 cytokine (I) and the IL-15Rα or a functional fragment thereof (S) are linked to the first half-life extension moiety (C); and in the other monomer: the masking polypeptide (MP) is linked to the second half-life extension moiety (C) through the cleavable moiety (CM).

In some embodiments, the IL-15 prodrug provided herein comprises two monomers, wherein in one monomer: the IL-15 cytokine (I) is linked to the first half-life extension moiety (C) and the IL-15Rα or a functional fragment thereof (S) is linked to the IL-15 cytokine (I); and in the other monomer: the masking polypeptide (MP) is linked to the second half-life extension moiety (C) through the cleavable moiety (CM).

In some embodiments, the IL-15 prodrug provided herein comprises two monomers, wherein in one monomer: the IL-15Rα or a functional fragment thereof (S) is linked to the first half-life extension moiety (C) and the IL-15 cytokine (I) is linked to the IL-15Rα or a functional fragment thereof (S); and in the other monomer: the masking polypeptide (MP) is linked to the second half-life extension moiety (C) through the cleavable moiety (CM).

In some embodiments, the IL-15 prodrug provided herein comprises two monomers, wherein in one monomer: the IL-15Rα or a functional fragment thereof (S) is linked to the first half-life extension moiety (C) and the masking polypeptide (MP) is linked to the first half-life extension moiety (C) through the cleavable moiety (CM); and in the other monomer: the IL-15 cytokine (I) is linked to the second half-life extension moiety (C).

In some embodiments, the IL-15 prodrug provided herein comprises two monomers, wherein in one monomer: the IL-15Rα or a functional fragment thereof (S) is linked to the first half-life extension moiety (C) and the masking polypeptide (MP) is linked to the IL-15Rα or a functional fragment thereof (S) through the cleavable moiety (CM); and in the other monomer: the IL-15 cytokine (I) is linked to the second half-life extension moiety (C).

In some embodiments, the IL-15 prodrug provided herein comprises two monomers, wherein in one monomer: the IL-15 cytokine (I) is linked to the first half-life extension moiety (C) and the masking polypeptide (MP) is linked to the first half-life extension moiety (C) through the cleavable moiety (CM); and in the other monomer: the IL-15Rα or a functional fragment thereof (S) is linked to the second half-life extension moiety (C).

In some embodiments, the IL-15 prodrug provided herein comprises two monomers, wherein in one monomer: the IL-15 cytokine (I) is linked to the first half-life extension moiety (C) and the masking polypeptide (MP) is linked to the IL-15 cytokine (I) through the cleavable moiety (CM); and in the other monomer: the IL-15Rα or a functional fragment thereof (S) is linked to the second half-life extension moiety (C).

In some embodiments, the IL-15 prodrug provided herein, wherein the IL-15Rα or a functional fragment thereof (S) and the IL-15 cytokine (I) are covalently linked; or the IL-15Rα or a functional fragment thereof (S) and the IL-15 cytokine (I) are non-covalently linked, and form an IL-15/IL-15Rα complex.

In some embodiments, the IL-15 prodrug provided herein, wherein the IL-15 cytokine (I) comprises the one or more amino acids mutations selected from the group consisting of L45D, L45E, Q48K, S51D, L52D, E64K, I67D, I67E, I68D and N72D.

In some embodiments, the IL-15 prodrug provided herein, wherein the IL-15 cytokine (I) comprises the amino acid sequence of any one of SEQ ID NOs: 22-23 and 67-76, or a variant thereof having at least about 90% (such as at least about any one of 91%, 92%, 93%, 94%, 95%, 96%, 97%, 98%, or 99%) sequence identity to the amino acid sequence of any one of SEQ ID NOs: 22-23 and 67-76.

In some embodiments, the IL-15 prodrug provided herein, wherein the IL-15Rα or a functional fragment thereof (S) comprises the amino acid sequence of any one of SEQ ID NOs: 24-26, or a variant thereof having at least about 90% (such as at least about any one of 91%, 92%, 93%, 94%, 95%, 96%, 97%, 98%, or 99%) sequence identity to the amino acid sequence of any one of SEQ ID NOs: 24-26.

In some embodiments, the IL-15 prodrug provided herein, wherein the half-life extension moiety (C) comprises an Fc domain; preferably, the Fc domain is selected from the group consisting of a human IgG1 Fc domain, a human IgG2 Fc domain, a human IgG3 Fc domain, a human IgG4 Fc domain, an IgA Fc domain, an IgD Fc domain, an IgE Fc domain, and an IgM Fc domain; more preferably, the Fc domain is a human IgG1 Fc domain.

In some embodiments, the IL-15 prodrug provided herein, wherein the Fc domain is a human IgG1 Fc domain having L234A and L235A mutations, according to EU Numbering system.

In some embodiments, the IL-15 prodrug provided herein, wherein the Fc domains comprises knobs-into-holes mutations (Fc knob and Fc hole).

In some embodiments, the IL-15 prodrug provided herein, wherein the Fc knob comprises a T366W mutation in the Fc domain, and the Fc hole comprises T366S, L368A, and Y407V mutations in the Fc domain, according to EU Numbering system.

In some embodiments, the IL-15 prodrug provided herein, wherein the Fc knob further comprises S354C mutation, and the Fc hole further comprises Y349C mutation, according to EU Numbering system.

In some embodiments, the IL-15 prodrug provided herein, wherein the masking polypeptide (MP) is composed of four or five types of amino acid residues selected from a group consisting of proline (P), alanine (A), serine (S), glycine (G), and glutamic acid (E).

In some embodiments, the IL-15 prodrug provided herein, wherein the masking polypeptide (MP) is composed of five types of amino acids G, S, P, E, and A, and further wherein the percentage of amino acid residue G in the masking polypeptide is about 15%-30%, preferably about 20%; the percentage of amino acid residue S in the masking polypeptide is about 20%-40%, preferably about 40%; the percentage of amino acid residue P in the masking polypeptide is about 15%-40%, preferably about 20%; the percentage of amino acid residue E in the masking polypeptide is about 1%-20%, preferably about 10%; and the percentage of amino acid residue A in the masking polypeptide is about 5%-20%, preferably about 10%; and when the number of amino acids is not an integer, take the integer value.

In some embodiments, the IL-15 prodrug provided herein, wherein the masking polypeptide (MP) is composed of four types of amino acids S, P, E, and G, and further wherein the percentage of amino acid residue S in the masking polypeptide is about 20%-40%, preferably about 23%; the percentage of amino acid residue P in the masking polypeptide is about 15%-40%, preferably about 29%, the percentage of amino acid residue E in the masking polypeptide is about 1%-20%, preferably about 18%, and the percentage of amino acid residue G in the masking polypeptide is about 15%-30%, preferably about 30%; and when the number of amino acids is not an integer, take the integer value.

In some embodiments, the IL-15 prodrug provided herein, wherein the masking polypeptide (MP) comprises about 40 to 720 amino acid residues; preferably comprises 80 to 320 amino acid residues; and more preferably comprises 80 to 240 amino acid residues.

In some embodiments, the IL-15 prodrug provided herein, wherein the masking polypeptide (MP) comprises the amino acid sequence of SEQ ID NO: 6.

In some embodiments, the IL-15 prodrug provided herein, wherein the masking polypeptide (MP) comprises the amino acid sequence of SEQ ID NO: 1.

In some embodiments, the IL-15 prodrug provided herein, wherein the masking polypeptide (MP) comprises the amino acid sequence of any one of SEQ ID NOs: 1-5, or a variant thereof having at least about 90% (such as at least about any one of 91%, 92%, 93%, 94%, 95%, 96%, 97%, 98%, or 99%) sequence identity to the amino acid sequence of any one of SEQ ID NOs: 1-5, or a variant thereof comprising one or more amino acid substitutions, additions and/or deletions.

In some embodiments, the IL-15 prodrug provided herein, wherein the cleavable moiety (CM) comprises the amino acid sequence MVX₁X₂AX₃TX₄SG (SEQ ID NO: 49), wherein X₁ is selected from P, L, V, or A, X₂ is selected from L or S, X₃ is selected from L V, P, or Y and X₄ is selected from A or V.

In some embodiments, the IL-15 prodrug provided herein, wherein the cleavable moiety (CM) is a substrate of urokinase-type plasminogen activator (uPA), matrix metallopeptidase (MMP) 1, MMP2, MMP3, MMP4, MMP5, MMP6, MMP7, MMP8, MMP9, MMP10, MMP11, MMP12, MMP13, MMP14, fibroblast activation protein (FAP), matriptase, cathepsin, caspase, thrombin, metalloprotease, serine protease, cysteine protease, aspartic acid protease, Legumain, Kallikrein, Cathepsin A, Cathepsin B, chymase, protease located at a tumor site or its surrounding environment or any combination thereof.

In some embodiments, the IL-15 prodrug provided herein, wherein the cleavable moiety (CM) comprises the amino acid sequence of any one of SEQ ID NOs: 8-16, or a variant thereof having at least about 90% (such as at least about any one of 91%, 92%, 93%, 94%, 95%, 96%, 97%, 98%, or 99%) sequence identity to the amino acid sequence of any one of SEQ ID NOs: 8-16.

In some embodiments, the IL-15 prodrug provided herein comprises two monomers, wherein in one monomer, the IL-15Rα or a functional fragment thereof (S) is linked to the first Fc domain, and in the other monomer, the IL-15 cytokine (I) is linked to the second Fc domain, and the masking polypeptide (MP) is linked to the IL-15 cytokine (I) through the cleavable moiety (CM).

In some embodiments, the IL-15 prodrug provided herein comprises two monomers, wherein one monomer comprises the amino acid sequence of SEQ ID NO: 33 or a variant thereof having at least about 80% (such as at least about any one of 80%, 85%, 90%, 95%, 96%, 97%, 98%, or 99%) sequence identity to the amino acid sequence of SEQ ID NO: 33, and the other monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein one monomer comprises the amino acid sequence of SEQ ID NO: 34 or a variant thereof having at least about 80% (such as at least about any one of 80%, 85%, 90%, 95%, 96%, 97%, 98%, or 99%) sequence identity to the amino acid sequence of SEQ ID NO: 34, and the other monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein one monomer comprises the amino acid sequence of SEQ ID NO: 36 or a variant thereof having at least about 80% (such as at least about any one of 80%, 85%, 90%, 95%, 96%, 97%, 98%, or 99%) sequence identity to the amino acid sequence of SEQ ID NO: 36, and the other monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein one monomer comprises the amino acid sequence of SEQ ID NO: 37 or a variant thereof having at least about 80% (such as at least about any one of 80%, 85%, 90%, 95%, 96%, 97%, 98%, or 99%) sequence identity to the amino acid sequence of SEQ ID NO: 37, and the other monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein one monomer comprises the amino acid sequence of SEQ ID NO: 38 or a variant thereof having at least about 80% (such as at least about any one of 80%, 85%, 90%, 95%, 96%, 97%, 98%, or 99%) sequence identity to the amino acid sequence of SEQ ID NO: 38, and the other monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein one monomer comprises the amino acid sequence of SEQ ID NO: 39 or a variant thereof having at least about 80% (such as at least about any one of 80%, 85%, 90%, 95%, 96%, 97%, 98%, or 99%) sequence identity to the amino acid sequence of SEQ ID NO: 39, and the other monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein one monomer comprises the amino acid sequence of SEQ ID NO: 45 or a variant thereof having at least about 80% (such as at least about any one of 80%, 85%, 90%, 95%, 96%, 97%, 98%, or 99%) sequence identity to the amino acid sequence of SEQ ID NO: 45, and the other monomer comprises the amino acid sequence of SEQ ID NO: 46 or a variant thereof having at least about 80% (such as at least about any one of 80%, 85%, 90%, 95%, 96%, 97%, 98%, or 99%) sequence identity to the amino acid sequence SEQ ID NO: 46.

In some embodiments, the IL-15 prodrug provided herein comprises two monomers, wherein one monomer comprises the amino acid sequence of SEQ ID NO: 47 or a variant thereof having at least about 80% (such as at least about any one of 80%, 85%, 90%, 95%, 96%, 97%, 98%, or 99%) sequence identity to the amino acid sequence of SEQ ID NO: 47, and the other monomer comprises the amino acid sequence of SEQ ID NO: 40 or a variant thereof having at least about 80% (such as at least about any one of 80%, 85%, 90%, 95%, 96%, 97%, 98%, or 99%) sequence identity to the amino acid sequence SEQ ID NO: 40.

In some embodiments, the IL-15 prodrug provided herein comprises two monomers, wherein the first monomer comprises the amino acid sequence of SEQ ID NO: 33 or a variant thereof having at least about 80% (such as at least about any one of 80%, 85%, 90%, 95%, 96%, 97%, 98%, or 99%) sequence identity to the amino acid sequence of SEQ ID NO: 33, and the second monomer comprises the amino acid sequence of SEQ ID NO: 40 or a variant thereof having at least about 80% (such as at least about any one of 80%, 85%, 90%, 95%, 96%, 97%, 98%, or 99%) sequence identity to the amino acid sequence SEQ ID NO: 40.

In some embodiments, the IL-15 prodrug provided herein comprises two monomers, wherein the first monomer comprises the amino acid sequence of SEQ ID NO: 62 or a variant thereof having at least about 80% (such as at least about any one of 80%, 85%, 90%, 95%, 96%, 97%, 98%, or 99%) sequence identity to the amino acid sequence of SEQ ID NO: 62, and the second monomer comprises the amino acid sequence of SEQ ID NO: 63 or a variant thereof having at least about 80% (such as at least about any one of 80%, 85%, 90%, 95%, 96%, 97%, 98%, or 99%) sequence identity to the amino acid sequence SEQ ID NO: 63.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 63 or a variant thereof having at least about 80% (such as at least about any one of 80%, 85%, 90%, 95%, 96%, 97%, 98%, or 99%) sequence identity to the amino acid sequence SEQ ID NO: 63.

In some embodiments, the IL-15 prodrug provided herein comprises two monomers, wherein the first monomer comprises the amino acid sequence of SEQ ID NO: 64 or a variant thereof having at least about 80% (such as at least about any one of 80%, 85%, 90%, 95%, 96%, 97%, 98%, or 99%) sequence identity to the amino acid sequence of SEQ ID NO: 64, and the second monomer comprises the amino acid sequence of SEQ ID NO: 65 or a variant thereof having at least about 80% (such as at least about any one of 80%, 85%, 90%, 95%, 96%, 97%, 98%, or 99%) sequence identity to the amino acid sequence SEQ ID NO: 65.

In some embodiments, the IL-15 prodrug provided herein comprises two monomers, wherein the first monomer comprises the amino acid sequence of SEQ ID NO: 62 or a variant thereof having at least about 80% (such as at least about any one of 80%, 85%, 90%, 95%, 96%, 97%, 98%, or 99%) sequence identity to the amino acid sequence of SEQ ID NO: 62, and the second monomer comprises the amino acid sequence of SEQ ID NO: 65 or a variant thereof having at least about 80% (such as at least about any one of 80%, 85%, 90%, 95%, 96%, 97%, 98%, or 99%) sequence identity to the amino acid sequence SEQ ID NO: 65.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 65 or a variant thereof having at least about 80% (such as at least about any one of 80%, 85%, 90%, 95%, 96%, 97%, 98%, or 99%) sequence identity to the amino acid sequence SEQ ID NO: 65.

In some embodiments, the IL-15 prodrug provided herein comprises two monomers, wherein the first monomer comprises the amino acid sequence of SEQ ID NO: 66 or a variant thereof having at least about 80% (such as at least about any one of 80%, 85%, 90%, 95%, 96%, 97%, 98%, or 99%) sequence identity to the amino acid sequence of SEQ ID NO: 66, and the second monomer comprises the amino acid sequence of SEQ ID NO: 65 or a variant thereof having at least about 80% (such as at least about any one of 80%, 85%, 90%, 95%, 96%, 97%, 98%, or 99%) sequence identity to the amino acid sequence SEQ ID NO: 65.

In some embodiments, the IL-15 prodrug provided herein comprises two monomers, wherein the first monomer comprises the amino acid sequence of SEQ ID NO: 43 or a variant thereof having at least about 80% (such as at least about any one of 80%, 85%, 90%, 95%, 96%, 97%, 98%, or 99%) sequence identity to the amino acid sequence of SEQ ID NO: 43, and the second monomer comprises the amino acid sequence of SEQ ID NO: 46 or a variant thereof having at least about 80% (such as at least about any one of 80%, 85%, 90%, 95%, 96%, 97%, 98%, or 99%) sequence identity to the amino acid sequence SEQ ID NO: 46.

In some embodiments, the IL-15 prodrug provided herein comprises two monomers, wherein the first monomer comprises the amino acid sequence of SEQ ID NO: 39 or a variant thereof having at least about 80% (such as at least about any one of 80%, 85%, 90%, 95%, 96%, 97%, 98%, or 99%) sequence identity to the amino acid sequence of SEQ ID NO: 39, and the second monomer comprises the amino acid sequence of SEQ ID NO: 40 or a variant thereof having at least about 80% (such as at least about any one of 80%, 85%, 90%, 95%, 96%, 97%, 98%, or 99%) sequence identity to the amino acid sequence SEQ ID NO: 40.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 87 or a variant thereof having at least about 80% (such as at least about any one of 80%, 85%, 90%, 95%, 96%, 97%, 98%, or 99%) sequence identity to the amino acid sequence SEQ ID NO: 87.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 91 or a variant thereof having at least about 80% (such as at least about any one of 80%, 85%, 90%, 95%, 96%, 97%, 98%, or 99%) sequence identity to the amino acid sequence SEQ ID NO: 91.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 93 or a variant thereof having at least about 80% (such as at least about any one of 80%, 85%, 90%, 95%, 96%, 97%, 98%, or 99%) sequence identity to the amino acid sequence SEQ ID NO: 93.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 94 or a variant thereof having at least about 80% (such as at least about any one of 80%, 85%, 90%, 95%, 96%, 97%, 98%, or 99%) sequence identity to the amino acid sequence SEQ ID NO: 94.

In some embodiments, also provided is the IL-15 drug comprises two monomers, wherein in one monomer, the IL-15Rα or a functional fragment thereof (S) is linked to the first Fc domain, and in the other monomer, the IL-15 cytokine (I) is linked to the second Fc domain.

In some embodiments, the IL-15 drug provided herein comprises two monomers, wherein the first monomer comprises the amino acid sequence of SEQ ID NO: 43 or a variant thereof having at least about 80% (such as at least about any one of 80%, 85%, 90%, 95%, 96%, 97%, 98%, or 99%) sequence identity to the amino acid sequence of SEQ ID NO: 43, and the second monomer comprises the amino acid sequence of SEQ ID NO: 83 or a variant thereof having at least about 80% (such as at least about any one of 80%, 85%, 90%, 95%, 96%, 97%, 98%, or 99%) sequence identity to the amino acid sequence SEQ ID NO: 83.

In some embodiments, the IL-15 drug provided herein comprises two monomers, wherein the first monomer comprises the amino acid sequence of SEQ ID NO: 43 or a variant thereof having at least about 80% (such as at least about any one of 80%, 85%, 90%, 95%, 96%, 97%, 98%, or 99%) sequence identity to the amino acid sequence of SEQ ID NO: 43, and the second monomer comprises the amino acid sequence of SEQ ID NO: 84 or a variant thereof having at least about 80% (such as at least about any one of 80%, 85%, 90%, 95%, 96%, 97%, 98%, or 99%) sequence identity to the amino acid sequence SEQ ID NO: 84.

Also provided are isolated nucleic acid molecule encoding any of the IL-15 prodrugs or drugs provided herein, vectors comprising such nucleic acid molecule, host cell (e.g., CHO cells, HEK 293 cells, Hela cells, or COS cells) comprising such nucleic acids or vectors, compositions (e.g., pharmaceutical compositions), kits, and articles of manufacture comprising any of the masking polypeptides, cleavable moieties, prodrugs, or drugs provided herein. Methods of treating a disease (e.g., a tumor) in an individual (e.g., human) using any of the IL-15 prodrugs or drugs provided herein or pharmaceutical compositions thereof are also provided.

### BRIEF DESCRIPTION OF THE DRAWINGS

FIG. 1A depicts the alignment of the amino acid sequences of MP80 and MP100, the amino acid sequence marked in the box (the amino acid sequence marked in the first box is SEQ ID NO: 6) are identical between MP80 and MP100.

FIG. 1B depicts the alignment of the amino acid sequences of two repeats of MP80 and MP163, the amino acid sequence marked in the first box was the sequence of MP80, i.e., the MP163 comprises the amino acid sequence of MP80.

FIG. 1C depicts the alignment of the amino acid sequences of triple repeats of MP80 and MP240, the amino acid sequence marked in the first box was the sequence of MP80, the MP240 comprises 3 copies of the amino acid sequence of MP80.

FIG. 2A depicts an exemplary IL-15 prodrug with an Fc domain as a half-life extension moiety, showing that an IL-15Rα_sushi domain linked to the C-terminus of one Fc domain, optionally through a non-cleavable linker. An IL-15 is linked to the C-terminus of the other Fc domain, optionally through a non-cleavable linker. A masking polypeptide (MP) is linked to the IL-15 through a cleavable moiety (CM).

FIG. 2B is an exemplary schematic drawing illustrating the activation process of IL-15 prodrug by released off the masking polypeptide (MP) at the target tissue (e.g., tumor with high levels of MMPs).

FIG. 3 depicts photograph of non-reduced and reduced SDS-PAGE gels analyzing the purity of exemplary prodrugs SB1902-C2 and SB1902-C7, and drug SB1902-C1 (without masking polypeptide).

FIG. 4 depicts graphs of SEC-HPLC analyzing the homogeneity of exemplary prodrug SB1902-C2, and drug SB1902-C1 (without masking polypeptide).

FIG. 5A shows the photograph of polyacrylamide gel analyzing the exemplary cleavable moiety in prodrug SB1902-C2 which is sensitive to the MMP2 enzyme.

FIG. 5B shows the photograph of polyacrylamide gel analyzing the exemplary cleavable moiety in prodrug SB1902-C2 which is sensitive to the MMP9 enzyme.

FIG. 5C shows the photograph of polyacrylamide gel analyzing the SB1902-C4 with a non-cleavable (G4S)2 linker instead of cleavable moiety, which is kept intact after enzyme MMP2 digestion.

FIG. 5D shows the photograph of polyacrylamide gel analyzing SB1902-C4 with a non-cleavable (G4S)2 linker instead of cleavable moiety, which is kept intact after enzyme MMP9 digestion.

FIG. 5E shows the photograph of polyacrylamide gel analyzing the exemplary cleavable moiety in prodrug SB1902-C5 which is sensitive to the MMP9 enzyme.

FIG. 5F shows the photograph of polyacrylamide gel analyzing the exemplary cleavable moiety in prodrug SB1902-C5 which is sensitive to the MMP2 enzyme.

FIG. 6A depicts that the drug SB1902-C1 (without masking polypeptide and cleavable moiety), prodrug SB1902-C2, prodrug SB1902-C3, and SB1902-C4 (without cleavable moiety) show no non-specific binding with human serum protein.

FIG. 6B depicts that the drug SB1902-C1 (without masking polypeptide), prodrug SB1902-C2, prodrug SB1902-C3, and SB1902-C4 (without cleavable moiety) show no non-specific binding with cynomolgus monkey serum protein.

FIG. 6C depicts that the drug SB1902-C1 (without masking polypeptide), prodrug SB1902-C2, prodrug SB1902-C3, and SB1902-C4 (without cleavable moiety) show no non-specific binding with rat serum protein.

FIG. 6D depicts the bands of prodrug SB1902-C2, prodrug SB1902-C3, and SB1902-C4 (without cleavable moiety) on WB membrane before incubation with plasma or in PBS buffer.

FIG. 6E shows no detectable degradation bands on the WB membrane for both prodrug SB1902-C2, prodrug SB1902-C3, and SB1902-C4 (without cleavable moiety) after incubated with human plasma (abbreviated as plas in the figure) or in PBS buffer.

FIG. 7A shows the binding affinity of the exemplary prodrug SB1902-C2 and the exemplary drug SB1902-C1 to IL-2/IL-15Rβγ receptor; FIGS. 7B-7D show the binding affinity of the exemplary IL-15 drug SB1902-C1-variant3 with wild-type IL-15 and IL-15 variants to IL-2/IL-15Rβγ receptor; FIGS. 7E-7F show the binding affinity of the exemplary IL-15 prodrug SB1902-C9-variant2 with wild-type IL-15 and IL-15 variants to IL-2/IL-15Rβγ receptor.

FIG. 8 depicts the results of the immunogenicity assessment assay in Balbc mice, showing that the MP80 masking polypeptide is not immunogenic.

FIG. 9 depicts the results of the Mo7e cell proliferation assay, showing that the prodrug SB1902-C2, SB1902-C6, and SB1902-C7 significantly reduced the IL-15 function in stimulating Mo7e cell proliferation as compared to the drug SB1902-C1.

FIGS. 10A-10Q depict the results of the exemplary IL-15 drugs and IL-15 prodrugs in CD8+ T cell activation assay.

FIG. 11 shows the result of the exemplary prodrug SB1902-C2 and MMP-digested SB1902-C2 of which the masking polypeptide was removed off, in CD8+ T cell activation assay.

FIG. 12A depicts the results of the IFN-γ production assay in PBMCs, FIG. 12B depicts the results of the Granzyme B production assay in PBMCs. FIG. 12C and FIG. 12D depict the results of the IL-15 drug and IL-15 prodrug with wild-type IL-15 and IL-15 variants in IFN-γ production assay. FIG. 12E and FIG. 12F depict the results of the IL-15 drug and IL-15 prodrug with wild-type IL-15 and IL-15 variants in Granzyme B production assay.

FIG. 13 depicts the results of the IFN-γ production assay in Balb/c mouse, showing that mice treated with the prodrug SB1902-C2 have less IFN-γ production compared to the drug SB1902-C1.

FIGS. 14A-14B show that the animals with WEHI-164 tumor were treated with IgG1 isotype control antibody MOPC-21 (FIG. 14A) or prodrug SB1902-C2 (FIG. 14B) at a dose of 3 mg/kg, the tumor growth in SB19020-C2 treated animals was dramatically inhibited compared to the tumor growth in animals treated with isotype control antibody.

FIGS. 14C-14H show the anti-tumor activity of the different compounds in animals with WEHI-164 tumor that were treated with IgG1 isotype control antibody MOPC-21 (FIG. 14C), SB1902-C4 without cleavable moiety (FIG. 14D), drug SB1902-C1 (FIG. 14E), prodrug SB1902-C2 (FIG. 14F), prodrug SB1902-C5 (FIG. 14G), or prodrug SB1902-C3 (FIG. 14H) at a dose of 1 mg/kg, except for drug SB1902-C1 group at a dose of 0.3 mg/kg, at day 0, 4, 7, 10, and 14.

FIG. 15A shows the illustrative structure of the anti-TNFR2 antibody, FIGS. 15B-15D show the illustrative structure of masked antibody prodrug Pepbody-SB1901-H, Pepbody-SB1901-L and Pepbody-SB1901-HL respectively.

FIG. 16A shows the antigen binding results of the exemplary anti-TNFR2 antibody prodrugs and anti-TNFR2 antibody.

FIG. 16B shows the results of the exemplary anti-TNFR2 antibody prodrug Pepbody-SB1901-H in human primary Treg cell proliferation assay compared with the anti-TNFR2 antibody SB1901-72.

### DETAILED DESCRIPTION

Disclosed herein are IL-15 prodrugs with masking polypeptides (MP) and the cleavable moiety (CM). The IL-15 prodrugs overcome the toxicity that have severely limited the clinical use of IL-15. The activity of IL-15 in the prodrugs is attenuated. The cleavable moiety in the prodrug includes protease cleave sites, the masking polypeptides in the prodrug is cleaved off by proteases that are associated with the desired site, such as in a tumor or tumor microenvironment, to recover the activity of IL-15.

### Definitions

The practice of the present application will employ, unless indicated specifically to the contrary, conventional methods of virology, immunology, microbiology, molecular biology, and recombinant DNA techniques within the skills of the art, many of which are described below for illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3rd ed., John Wiley & Sons, 1995; Sambrook and Russell, Molecular Cloning: A Laboratory Manual (3rd Edition, 2001); Maniatis et al., Molecular Cloning: A Laboratory Manual (1982); DNA Cloning: A Practical Approach, vol. I&II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

As used herein, “treatment” or “treating” is an approach for obtaining beneficial or desired results including clinical results. For purposes of this application, beneficial or desired results including clinical results, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of a pathological consequence of the disease. The methods of the application contemplate any one or more of these aspects of treatment. For example, an individual is successfully “treated” if one or more symptoms associated with the disease are mitigated or eliminated, including, but are not limited to, decreasing symptoms resulting from the disease, increasing the quality of life of those suffering from the disease, decreasing the dose of other medications required to treat the disease, and/or prolonging survival of individuals.

The term “prevent,” and similar words such as “prevented,” “preventing” etc., indicate an approach for preventing, inhibiting, or reducing the likelihood of the occurrence or recurrence of, a disease or condition. It also refers to delaying the occurrence or recurrence of a disease or condition or delaying the occurrence or recurrence of the symptoms of a disease or condition. As used herein, “prevention” and similar words also include reducing the intensity, effect, symptoms, and/or burden of a disease or condition prior to recurrence of the disease or condition.

As used herein, “delaying” the development of a disease means to defer, hinder, slow, retard, stabilize, and/or postpone development of the disease. This delay can be of varying lengths of time, depending on the history of the disease and/or individual being treated. A method that “delays” development of a disease is a method that reduces probability of disease development in a given time frame and/or reduces the extent of the disease in a given time frame, when compared to not using the method. Such comparisons are typically based on clinical studies, using a statistically significant number of individuals.

The term “effective amount” used herein refers to an amount of an agent or a combination of agents, sufficient to treat a specified disorder, condition, or disease such as ameliorate, palliate, lessen, and/or delay one or more of its symptoms. In some embodiments, an effective amount is an amount sufficient to delay disease development. In some embodiments, an effective amount is an amount sufficient to prevent or delay disease recurrence. An effective amount can be administered in one or more administrations. In the case of a disease such as cancer, an effective amount may be an amount sufficient to delay cancer development or progression (e.g., decrease tumor growth rate, and/or delay or prevent tumor angiogenesis, metastasis, or infiltration of cancer cells into peripheral organs), reduce the number of epithelioid cells, cause cancer regression (e.g., shrink or eradicate a tumor), and/or prevent or delay cancer occurrence or recurrence. An effective amount can be administered in one or more administrations.

As used herein, an “individual” or a “subject” refers to a mammal, including, but not limited to, human, bovine, horse, feline, canine, rodent, or primate. In some embodiments, the individual is a human.

The term “antibody” includes full-length antibodies and antigen-binding fragments thereof. In some embodiments, a full-length antibody comprises two heavy chains and two light chains. The variable regions of the light and heavy chains are responsible for antigen binding.

The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgAQ1 (α1 heavy chain), or IgA2 (α2 heavy chain).

The term “antigen-binding fragment” as used herein includes an antibody fragment including, for example, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a VHH, a single-chain Fv (scFv), an scFv dimer (bivalent diabody), a multispecific antibody formed from a portion of an antibody comprising one or more CDRs, a single domain antibody, a nanobody, a domain antibody, a bivalent domain antibody, or any other antibody fragments that bind to an antigen but do not comprise a complete antibody structure. An antigen-binding fragment also includes a fusion protein comprising the antibody fragment described above. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al, J. Mol. Biol. 196:901-917 (1987); Al-Lazikani B. et al., J. Mol. Biot, 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2, and C_{H}3 domains (collectively, C_{H}) of the heavy chain and the C_{L} domain of the light chain. Depending on the amino acid sequence of the constant domain of immunoglobulin heavy chains (C_{H}), immunoglobulins can be assigned to different classes or isotypes. There are five classes of immunoglobulins: IgA, IgD, IgE, IgG, and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgAQ1, and IgA2.

The term “Fc,” “Fc region,” “fragment crystallizable region,” “Fc domain,” or “Fc moiety” herein is used to define a C-terminal region of an immunoglobulin heavy chain, including native-sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230 to the carboxyl-terminus thereof. The C-terminal lysine (residue 447 according to the EU numbering system) of the Fc region may be removed, for example, during production or purification of the protein, or by recombinantly engineering the nucleic acid encoding the protein. Suitable native-sequence Fc regions for use in the constructs described herein include human IgG1, IgG2 (IgG2A, IgG2B), IgG3, and IgG4.

The term IgG “isotype” or “subclass” as used herein is meant any of the subclasses of immunoglobulins defined by the chemical and antigenic characteristics of their constant regions. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000).

“Fc receptor” or “FcR” describes a receptor that binds the Fc region of an Fc-containing construct (e.g., antibody, or protein containing Fc region, referred to as Fc fusion protein hereafter). The preferred FcR is a native sequence of human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Dacron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

“Antibody effector functions” refer to those biological activities attributable to the Fc region (a native sequence Fc region or amino acid sequence variant Fc region) of an Fc-containing construct (e.g., antibody or Fc fusion protein), and vary with Fc isotype. Examples of antibody effector functions include C1q binding and complement dependent cytotoxicity (CDC); Fc receptor binding; antibody-dependent cell-mediated cytotoxicity (ADCC); phagocytosis; down regulation of cell surface receptors (e.g., B cell receptors); and B cell activation. “Reduced or minimized” antibody effector function means that which is reduced by at least 50% (alternatively 60%, 65%, 70%, 75%, 80%, 85%, 90%, 95%, 96%, 97%, 98%, or 99%) from the wild type or unmodified Fc-containing construct (e.g., antibody or Fc fusion protein). The determination of antibody effector function is readily determinable and measurable by one of ordinary skill in the art. In a preferred embodiment, the antibody effector functions of complement binding, complement dependent cytotoxicity and antibody dependent cytotoxicity are affected. In some embodiments, effector function is eliminated through a mutation in the constant region that eliminated glycosylation, e.g., “effectless mutation.” In some embodiments, the effectless mutation is an N297A or DANA mutation (D265A+N297A) in the C_{H}2 region. Shields et al., J. Biol. Chem. 276 (9): 6591-6604 (2001). Alternatively, additional mutations resulting in reduced or eliminated effector function include K322A and L234A/L235A (LALA). Alternatively, effector function can be reduced or eliminated through production techniques, such as expression in host cells that do not glycosylate (e.g., E. coli.) or in which result in an altered glycosylation pattern that is ineffective or less effective at promoting effector function (e.g., Shinkawa et al., J. Biol. Chem. 278(5): 3466-3473 (2003).

“Antibody-dependent cell-mediated cytotoxicity” or ADCC refers to a form of cytotoxicity in which secreted Ig (or Ligand-Fc construct) bound onto Fc receptors (FcRs) present on certain cytotoxic cells (e.g., natural killer (NK) cells, neutrophils, and macrophages) enable these cytotoxic effector cells to bind specifically to an antigen-bearing (or ligand receptor-bearing) target cell and subsequently kill the target cell with cytotoxins. The antibodies (or Fc-containing constructs) “arm” the cytotoxic cells and are required for killing the target cell by this mechanism. The primary cells for mediating ADCC, NK cells, express FcγRIII only, whereas monocytes express FcγRI, FcγRII, and FcγRIII. Fc expression on hematopoietic cells is summarized in Table 2 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

“Complement dependent cytotoxicity” or “CDC” refers to the lysis of a target cell in the presence of complement. Activation of the classical complement pathway is initiated by the binding of the first component of the complement system (C1q) to Fc-containing constructs (of the appropriate subclass) which are bound to their cognate receptor through the ligand fused to Fc. To assess complement activation, a CDC assay, e.g., as described in Gazzano-Santoro et al., J. Immunol. Methods 202: 163 (1996), may be performed. Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

As used herein, the term “specifically binds,” “specifically recognizes,” or is “specific for” refers to measurable and reproducible interactions such as binding between a ligand and a receptor, which is determinative of the presence of the ligand in the presence of a heterogeneous population of molecules including biological molecules. For example, a ligand that specifically binds a receptor is a ligand that binds this receptor with greater affinity, avidity, more readily, and/or with greater duration than it binds other receptors. In some embodiments, the extent of binding of a ligand to an unrelated receptor is less than about 10% of the binding of the ligand to the target receptor as measured, e.g., by a radioimmunoassay (RIA). In some embodiments, a ligand that specifically binds a target receptor has an equilibrium dissociation constant (Kd) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, a ligand specifically binds a receptor that is conserved among the receptors from different species. In some embodiments, specific binding can include, but does not require exclusive binding. Binding specificity of a ligand can be determined experimentally by methods known in the art. Such methods comprise, but are not limited to Western blots, ELISA-, RIA-, ECL-, IRMA-, EIA-, BIACORE™-tests and peptide scans.

As used herein, the term “substrate” when used in reference to a protease (e.g., metalloproteinase) is intended to mean any material or substance on which the protease (e.g., metalloproteinase) acts. The material or substance can be, for example, a naturally or non-naturally occurring organic chemical, or a macromolecule such as a polypeptide or peptidomimetic. In some embodiments, a metalloproteinase substrate specifically interacts with one or more metalloproteinases, and is cleaved by the metalloproteinase. At least one molecule of the substrate is cleaved by the metalloproteinase using appropriate conditions within the time frame of an experiment. In some embodiments, 10%, 20%, 30%, 40%, 50%, 60%, 70%, 80%, 90%, or 100% of the substrate can be cleaved by the metalloproteinase

The term “functional analog” refers to a molecule that has the same biological specificity (e.g., binding to the same ligand) and/or activity (e.g., activating or inhibiting a target cell) as a reference molecule.

The term “prodrug” refers to a therapeutic molecule that is not active until being activated in vivo.

The term “modulate” includes “increase”, “enhance” or “stimulate” as well as “decrease” or “reduce”, typically in a statistically or physiologically significant amount or degree relative to a control.

The term “variant” comprises one or more substitutions, additions, deletions and/or insertions relative to a reference polypeptide or polynucleotide. A variant of a polypeptide or polynucleotide comprises an amino acid or nucleotide sequence having at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of the reference sequence. Also included are sequences that consist of or differ from a reference sequence by the addition, deletion, insertion or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids or nucleotides and that substantially retain at least one activity of the reference sequence. In certain embodiments, addition or deletion includes C-terminal and/or N-terminal addition and/or deletion.

The term “wild-type” refers to a gene or gene product (e.g., a polypeptide) that is most often observed in a population, and is thus set as the “normal” or “wild-type” form of the gene.

The term “linked” included covalently linked or non-covalently linked, referring to a first moiety, e.g., a first amino acid sequence or nucleotide sequence, covalently or non-covalently joined to a second moiety, e.g., a second amino acid sequence or nucleotide sequence, respectively. The first moiety can be directly joined or juxtaposed to the second moiety (referred to as directly linked, e.g., through peptide bond in the case of polypeptides) or, alternatively, intervening moiety (e.g., peptide linker) can be used to join the first moiety to the second moiety (referred to as indirectly linked), which can be said that the first moiety is linked to the second moiety through intervening moiety. In the case of polypeptides or proteins, the term “linked” not only includes a linkage of a first moiety (or a second moiety) at the C-terminus and/or the N-terminus, but also includes the linkage of the whole first moiety (or the second moiety) to any positions (e.g. amino acid residues not located in the terminal) of the second moiety (or the first moiety, respectively). In one aspect, the first moiety is linked to a second moiety by a peptide bond or a linker. In some embodiments, the first moiety can be linked to a second moiety by a phosphodiester bond or a linker. In some embodiments, the term “linker” is recognized as and refers to a molecule (including but not limited to unmodified or modified nucleic acids or amino acids) or group of molecules (for example, 2 or more, e.g., 2, 3, 4, 10, 30, 50, 100 or more) or any chemical moiety connecting two moieties, such as two polypeptides.

“Covalent bond” as used herein refers to a stable bond between two atoms sharing one or more electrons. Examples of covalent bonds include, but are not limited to, peptide bonds and disulfide bonds. As used herein, “peptide bond” refers to a covalent bond formed between a carboxyl group of an amino acid and an amine group of an adjacent amino acid. A “disulfide bond” as used herein refers to a covalent bond formed between two sulfur atoms, such as a combination of two Fc fragments by one or more disulfide bonds. One or more disulfide bonds may be formed between the two fragments by linking the thiol groups in the two fragments. In some embodiments, one or more disulfide bonds can be formed between one or more cysteines of two Fc fragments. Disulfide bonds can be formed by oxidation of two thiol groups. In some embodiments, the covalent linkage is directly linked by a covalent bond. In some embodiments, the covalent linkage is directly linked by a peptide bond or a disulfide bond.

The term “fused” or “fusion” in reference to two polypeptide sequences refers to the joining of the two polypeptide sequences through a backbone peptide bond. Two polypeptides may be fused directly or through a peptide linker that comprises one or more amino acids. Fusion proteins are polypeptides that comprise two or more regions derived from different or heterologous, proteins or peptides. Fusion proteins are prepared using conventional techniques of enzyme cutting and ligation of fragments from, desired sequences. PCR techniques employing synthetic oligonucleotides may be used to prepare and/or amplify the desired fragments. Overlapping synthetic oligonucleotide representing the desired sequences can also be used to prepare DNA constructs encoding fusion proteins. Fusion proteins can comprise several sequences, including a leader (or signal peptide) sequence, linker sequence, a leucine zipper sequence, or other oligomer-forming sequences, and sequences encoding highly antigenic moieties that provide a means for facile purification or rapid detection of a fusion protein. A fusion protein may be made by recombinant technology from a coding sequence containing the respective coding sequences for the two fusion partners, with or without a coding sequence for a peptide linker in between. In some embodiments, fusion encompasses chemical conjugation.

The term “IL-15/IL-15Rα complex” provided herein refers to a complex in which the IL-15 cytokine and the IL-15Rα or a functional fragment are non-covalently linked with each other.

Half maximal inhibitory concentration (IC₅₀) is a measure of the effectiveness of a substance (e.g., ligand) in inhibiting a specific biological or biochemical function. It indicates how much of a particular drug or other substance (inhibitor, e.g., ligand) is needed to inhibit a given biological process by half. The values are typically expressed as molar concentration. IC₅₀ is comparable to an “EC₅₀” for agonist drug or other substance (e.g., ligand). EC₅₀ also represents the plasma concentration required for obtaining 50% of a maximum effect in vivo. As used herein, an “IC₅₀” is used to indicate the effective concentration of a ligand needed to neutralize 50% of the receptor bioactivity in vitro. IC₅₀ or EC₅₀ can be measured by bioassays such as inhibition of ligand binding by FACS analysis (competition binding assay), cell-based cytokine release assay, or amplified luminescent proximity homogeneous assay (AlphaLISA)

“Percent (%) amino acid sequence identity” and “homology” with respect to a peptide or polypeptide sequence are defined as the percentage of amino acid residues in a candidate sequence that is identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

As used herein, the “C-terminus” of a polypeptide refers to the last amino acid residue of the polypeptide which donates its amine group to form a peptide bond with the carboxyl group of its adjacent amino acid residue. “N-terminus” of a polypeptide as used herein refers to the first amino acid of the polypeptide which donates its carboxyl group to form a peptide bond with the amine group of its adjacent amino acid residue.

As used herein, the term “moiety” refers to a portion of a molecule that has a distinct function within that molecule, and that function may be performed by that moiety in the context of another molecule. A moiety may be a chemical entity with a particular function or a portion of a biological molecule with a particular function.

As used herein, the terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component. As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

An “isolated” polypeptide is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the polypeptide, and may include enzymes, hormones, and other proteinaceous or non-proteinaceous solutes. In some embodiments, the polypeptide will be purified: (1) to greater than 95% by weight of polypeptides as determined by, for example, the Lowry method, and in some embodiments, to greater than 99% by weight; (2) to a degree sufficient to obtain at least 15 residues of N-terminal or internal amino acid sequence by use of a spinning cup sequenator; or (3) to homogeneity by SDS-PAGE under non-reducing or reducing conditions using Coomassie Blue or, preferably, silver stain. Isolated polypeptide includes the polypeptide in situ within recombinant cells since at least one component of the polypeptide's natural environment will not be present. Ordinarily, however, an isolated polypeptide will be prepared by at least one purification step.

As used herein, the terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

An “isolated” nucleic acid molecule encoding a construct (such as the masking polypeptide described herein) is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides described herein are in a form other than in the form or setting in which it is found in nature. Isolated nucleic acid molecules therefore are distinguished from nucleic acid encoding the polypeptides described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one that has been transfected, transformed, or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that has the same function or biological activity as screened or selected for in the originally transformed cell are included herein.

The term “pharmaceutical formulation” or “pharmaceutical composition” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

It is understood that embodiments of the application described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “or,” and “the” include plural referents unless the context clearly dictates otherwise.

### Masking Polypeptide (MP)

The masking polypeptide (MP) as provided herein refers to a moiety capable of blocking the activity of the biologically active moiety (B). In some embodiments, the masking polypeptide (MP) can inhibit the ability of the cytokine to bind and/or activate its receptor. In some embodiments, the masking polypeptide (MP) can inhibit the ability of the antibody or antigen-binding fragment to bind to its target.

In some embodiments, the masking polypeptides (MP) have a larger hydrodynamic radius than their actual molecular weight. In some embodiments, the masking polypeptides only form a random coil, without a secondary structure. In some embodiments, the masking polypeptides have a steric masking effect that typically inhibits or blocks the activity of the biologically active moiety due to its proximity to the biologically active moiety and comparative size.

In some embodiments, the masking polypeptide (MP) is composed of four or five types of amino acid residues selected from a group consisting of proline (P), alanine (A), serine (S), glycine (G) and glutamic acid (E).

In some embodiments, the masking polypeptide (MP) is composed of four types of amino acid residues selected from a group consisting of proline (P), alanine (A), serine (S) and glutamic acid (E).

In some embodiments, the masking polypeptide (MP) is composed of four types of amino acid residues selected from a group consisting of proline (P), glycine (G), serine (S) and glutamic acid (E).

In some embodiments, the masking polypeptide (MP) is composed of five types of amino acid residues selected from a group consisting of proline (P), alanine (A), serine (S), glycine (G) and glutamic acid (E).

In some embodiments, the net charges in masking polypeptide (MP) are critical. In general, the negative charge is more preferred than positive charge. The net negative charge of MP could avoid its potential interaction with tissues and cell membranes, which could potentially ‘sink’ it if it has a net positive charge before it reaches the destination. However, too much negative charge could increase the interaction possibility with positively charged proteins. The percentage of the net charge is also depending on the fusion partner of MP.

In some embodiments, the masking polypeptide (MP) is composed of five types of amino acids G, S, P, E and A, and wherein the percentage of amino acid residue G in the masking polypeptide is about 15%-30%; the percentage of amino acid residue S in the masking polypeptide is about 20%-40%; the percentage of amino acid residue P in the masking polypeptide is about 15%-40%; the percentage of amino acid residue E in the masking polypeptide is about 1%-20%; and the percentage of amino acid residue A in the masking polypeptide is about 5%-20%; and when the number of amino acids is not an integer, take the integer value.

In some embodiments, the masking polypeptide (MP) is composed of five types of amino acids G, S, P, E and A, and wherein the percentage of amino acid residue G in the masking polypeptide is about 20%; the percentage of amino acid residue S in the masking polypeptide is about 40%; the percentage of amino acid residue P in the masking polypeptide is about 20%; the percentage of amino acid residue E in the masking polypeptide is about 10%; and the percentage of amino acid residue A in the masking polypeptide is about 10%; and when the number of amino acids is not an integer, take the integer value.

In some embodiments, the masking polypeptide (MP) is composed of four types of amino acids S, P, E and G, and wherein the percentage of amino acid residue S in the masking polypeptide is about 20%-40%; the percentage of amino acid residue P in the masking polypeptide is about 15%-40%; the percentage of amino acid residue E in the masking polypeptide is about 1%-20%; and the percentage of amino acid residue G in the masking polypeptide is about 15%-30%; and when the number of amino acids is not an integer, take the integer value.

In some embodiments, the masking polypeptide (MP) is composed of four types of amino acids S, P, E, and G, and wherein the percentage of amino acid residue S in the masking polypeptide is about 23%; the percentage of amino acid residue P in the masking polypeptide is about 29%; the percentage of amino acid residue E in the masking polypeptide is about 18%; and the percentage of amino acid residue G in the masking polypeptide is about 30%; and when the number of amino acids is not an integer, take the integer value.

In some embodiments, the masking polypeptide (MP) can be tuned by changing its amino acid chain length and its total net charge to meet certain requirements with certain partners.

In some embodiments, the masking polypeptides (MP) comprises about 40 to 720 amino acid residues. In some embodiments, the masking polypeptides (MP) comprises about 80 to 320 amino acid residues. In some embodiments, the masking polypeptides (MP) comprises about 80 to 240 amino acid residues.

In some embodiments, the masking polypeptide (MP) comprises the amino acid sequence of SEQ ID NO: 6.

In some embodiments, the masking polypeptide (MP) comprises the amino acid sequence of SEQ ID NO: 1.

In some embodiments, the masking polypeptide (MP) comprises the amino acid sequence of any one of SEQ ID NOs: 1-5, or a variant thereof having at least about 90% (such as at least about any one of 91%, 92%, 93%, 94%, 95%, 96%, 97%, 98%, or 99%) sequence identity to the amino acid sequence of any one of SEQ ID NOs: 1-5, or a variant thereof comprising one or more amino acid substitutions, additions and/or deletions

### Cleavable Moiety (CM)

The cleavable moiety (CM) is a polypeptide that comprises or is the cleavage site of an enzyme or a protease. In some embodiments, the proteases include but are not limited to urokinase-type plasminogen activator (uPA); matrix metalloproteinases (e.g., MMP1, MMP2, MMP3, MMP7, MMP8, MMP9, MMP10, MMP11, MMPP12, MMPP13, MMPP14, MMP15, MMP16, MMP17, MMP19, MMP20, MMP23, MMP24, MMP26, and/or MMP27); Tobacco Etch Virus (TEV) protease; plasmin; Thrombin; PSA; PSMA; ADAMS/ADAMTS (e.g., ADAM8, ADAM9, ADAM10, ADAM12, ADAM13, ADAM17/TACE, ADAMDEC1, ADAMTS1, ADAMTS4, and/or ADAMTS5); caspases (e.g., Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and/or Caspase-14); aspartate proteases (e.g., RACE and/or Renin); aspartic cathepsins (e.g., Cathepsin D and/or Cathepsin E); cysteine cathepsins (e.g., Cathepsin B, Cathepsin C, Cathepsin K, Cathepsin L, Cathepsin S, Cathepsin V/L2, and/or Cathepsin X/Z/P); cysteine proteinases (e.g., Cruzipain, Legumain, and/or Otubain-2); KLKs (e.g., KLK4, KLK5, KLK6, KLK7, KLK8, KLK10, KLK11, KLK13, and/or KLK14); metallo proteainases (e.g., Meprin, Neprilysin, PSMA, and/or BMP-1); serine proteases (e.g., activated protein C, Cathepsin A, Cathepsin G, Chymase, and/or coagulation factor proteases (such as FVIIa, FIXa, FXa, FXIa, FXIIa)); elastase; granzyme B; guanidinobenzoatase; HtrA1; human neutrophil elastase; lactoferrin; marapsin; NS3/4A; PACE4; tPA; tryptase; type II transmembrane serine proteases (TTSPs) (e.g., DESC1, DPP-4, FAP, Hepsin, Matriptase-2, MT-SP1/Matriptase, TMPRSS2, TMPRSS3 and/or TMPRSS4); etc.

In some embodiments, the cleavable moiety (CM) comprises a substrate sequence for at least one matrix metalloprotease (MMP). Examples of MMPs include MMP1; MMP2; MMP3; MMP7; MMP8; MMP9; MMP10; MMP11; MMP12; MMP13; MMP14; MMP15; MMP16; MMP17; MMP19; MMP20; MMP23; MMP24; MMP26; and MMP27. In some embodiments, the CM comprises a substrate sequence for MMP2, MMP9, MMP14, MMP1, MMP3, MMP13, MMP17, MMP11 and MMP19. In some embodiments, the CM comprises a substrate sequence for MMP2. In some embodiments, the CM comprises a substrate sequence for MMP9. In some embodiments, the CM comprises a substrate sequence for two or more MMPs. In some embodiments, the CM comprises a substrate sequence for at least MMP2 and MMP9. In some embodiments, the CM comprises two or more substrates for the same MMP. In some embodiments, the CM comprises at least two or more MMP2 substrates. In some embodiments, the CM comprises at least two or more MMP9 substrates.

In some embodiments, the cleavable moiety (CM) comprises the amino acid sequence MVX₁X₂AX₃TX₄SG (SEQ ID NO: 49), wherein X₁ is selected from P, L, V, or A, X₂ is selected from L or S, X₃ is selected from L V, P, or Y and X₄ is selected from A or V.

The specificity of a protease for cleavage of a peptide bond with particular amino acids in nearby positions is described in terminology based on that originally created by Schechter & Berger (1967, 1968) to describe the specificity of papain. According to this model, amino acid residues in a substrate undergoing cleavage are designated P1, P2, P3, P4 etc. in the N-terminal direction from the cleaved bond. Likewise, the residues in C-terminal direction are designated P1′, P2′, P3′, P4′. etc.

In some embodiments, the CM comprises the amino acid sequence of any one of SEQ ID NOs: 8-16, or a variant thereof having at least about 90% (such as at least about any one of 91%, 92%, 93%, 94%, 95%, 96%, 97%, 98%, or 99%) sequence identity to the amino acid sequence of any one of SEQ ID NOs: 8-16.

### Biologically Active Moiety (B)

In some embodiments, the biologically active moiety (B) can be a chemical entity. In some embodiments, the biologically active moiety (B) can be a therapeutic protein. In some embodiments, the biologically active moiety (B) is a cytokine. In some embodiments, the biologically active moiety (B) is an antibody or antigen-binding fragment that targets the prodrugs to a site of action (e.g., sites of inflammation, or a tumor).

Cytokines: “Cytokine” is a well-known term of art that refers to any of a class of immunoregulatory proteins (such as interleukin or interferon) that are secreted by cells especially of the immune system and that are modulators of the immune system. In some embodiments, the cytokine includes the functional fragment, mutant, or variant of the cytokine. Examples of cytokines may include chemokines, interferons, interleukins, lymphokines, and tumor necrosis factors. In the case of cytokine prodrugs, while the description below exemplifies IL-15 prodrugs. However, prodrugs for other cytokines, in particular cytokines that are potent immune regulators and have strong side effects, are also contemplated in the present disclosure. Upon proteolytic cleavage of the cleavable moiety at the target site, the cytokine becomes to be an activated form, which renders it capable of binding to its cognate receptor or protein with increased affinity. These other cytokine prodrugs may be made according to the same principles as illustrated below for IL-15 prodrugs.

In some embodiments, the cytokine is selected from the group consisting of IL-1α, IL-1β, IL-1 receptor antagonist (IL-1RA), IL-18, IL-33, IL-36α, IL-36β, IL-36γ, IL-36 receptor antagonist (IL-36RA), IL-37, and IL-38, or mutants of the cytokines.

In some embodiments, the cytokine is selected from the group consisting of IL-2, IL-3, IL-4, IL-5, IL-7, IL-9, IL-13, IL-15, IL-21, granulocyte-macrophage colony-stimulating factor (GM-CSF), macrophage colony-stimulating factor (M-CSF), IL-6, IL-11, IL-12, growth hormone (GH), erythropoietin (EPO), prolactin (PRL), leukemia inhibitory factor (LIF), oncostatin (OSM), and thrombopoietin (TPO), or mutants of the cytokines.

In some embodiments, the cytokine is selected from the group consisting of CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CCL1e, CCL2, CCL3, CCL3L1, CCL4, CCL5, CCL6, CCL7, CCL8, CCL9/10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CX3CL1, XCL1, and XCL2, or mutants of the cytokines.

In some embodiments, the cytokine is selected from the group consisting of IFN-α (alpha), IFN-β (beta), IFN-γ (gamma), IFN-ε (epsilon), IFN-κ (kappa), IFN-(ω) (omega), IFN-τ (tau), IFN-ζ (zeta), IFN-δ (delta), and IFN-λ, (lambda), or mutants of the cytokines.

In some embodiments, the cytokine is selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28A, IL-28B, IL-29, IL-30, IL-31, IL-32, IL-33, IL-34, IL-35, IL-36, and IL-37.

In some embodiments, the cytokine is selected from the group consisting of granulocyte-macrophage colony-stimulating factor (GM-CSF), macrophage colony-stimulating factor (M-CSF), tumor necrosis factor-alpha (TNF-α), transforming growth factor-beta (TGF-β), IFN-γ (gamma), IL-2, IL-3, IL-4, IL-5, IL-6, IL-7, IL-8, and IL-12.

In some embodiments, the cytokine is selected from the group consisting of TNF-α (alpha), TNF-β (beta), TNF-γ (gamma), CD252, CD154, CD178, CD70, CD153, 4-1BB-L, TRAIL, RANKL, APO3L, CD256, CD257, CD258, TL1, AITRL, and EDA1.

In some embodiments, the cytokine disclosed herein are mutated or engineered to alter the properties of the naturally occurring cytokine, including receptor binding affinity and specificity or serum half-life.

Antibodies: In the case of antibody prodrugs, while the description below exemplifies anti-TNFR2 antibody prodrugs, prodrugs for other antibodies, that are activated in the tumor microenvironment by tumor-associated proteases, thereby restricting the activity to the tumor microenvironment and minimizing “off-tumor” toxicity, in particular, antibodies that have less than optimal selectivity for the intended target are also contemplated in the present disclosure. In some embodiments, the masking polypeptide (MP) reduces the ability of the antibody or antigen-binding fragment thereof to bind the target, such that the dissociation constant (Kd) of the antibody or antigen-binding fragment coupled to the masking polypeptide (MP) towards the target is at least 10 times greater, at least 100 times greater, at least 1000 times greater, or at least 10,000 times greater than the Kd of the antibody or antigen-binding fragment not coupled to the masking polypeptide (MP) towards the target. Upon proteolytic cleavage of the cleavable moiety (CM) at the target site, the antibody or antigen-binding fragment becomes activated. Other antibody prodrugs may be made according to the same principles as illustrated below for anti-TNFR2 antibody prodrugs.

In some embodiments, the antibody or antigen-binding fragment is specific for regulatory T cells (Tregs), for example, targeting the CCR4, or CD39 receptors. In some embodiments, the specific antibody or antigen-binding fragment may bind to an antigen on the surface of an immune cell, for example, T cells, NK cells, and macrophages, for example, the specific antibody or antigen-binding fragment may bind to PD-1, LAG-3, TIM-3, TIGIT, CTLA-4, or TNF-α. In some embodiments, the specific antibody or antigen-binding fragment may have the ability to activate the immune cell and enhance its anti-cancer activity. In some embodiments, the specific antibody or antigen-binding fragment may bind to an antigen on the surface of a diseased cell, or tissue, for example, a tumor cell, the tumor antigens are well known in the art. The specific antibody or antigen-binding fragment that may bind a tumor antigen include but are not limited to Fibroblast activation protein alpha (FAPα), Trophoblast glycoprotein (5T4), Tumor-associated calcium signal transducer 2 (Trop2), Fibronectin EDB (EDB-FN), FOLR1, fibronectin EIIIB domain, CGS-2, EpCAM, EGFR, HER-2, HER-3, c-Met, and CEA.

In some embodiments, the specific antibody or antigen-binding fragment is specific for an immune checkpoint protein. Examples of immune checkpoint proteins include but are not limited to CD27, CD137, 2B4, TIGIT, CD155, ICOS, HVEM, CD40L, LIGHT, TIM-1, OX40, DNAM-1, PD-L1, PD-1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA.

In some embodiments, the specific antibody or antigen-binding fragment is specific for an immune response modulator. Examples of immune response modulators include but are not limited to granulocyte-macrophage colony-stimulating factor (GM-CSF), macrophage colony-stimulating factor (M-CSF), granulocyte colony-stimulating factor (G-CSF), B7-1 (CD80), B7-2 (CD86), GITRL, CD3, or GITR.

In some embodiments, the specific antibody or antigen-binding fragment is specific for a cytokine receptor. Examples of cytokine rectors include but are not limited to Type I cytokine receptors, such as GM-CSF receptor, G-CSF receptor, Type I IL receptors, Epo receptor, LIF receptor, CNTF receptor, TPO receptor; Type II Cytokine receptors, such as IFN-alpha receptor (IFNAR1, IFNAR2), IFB-beta receptor, IFN-gamma receptor (IFNGR1, IFNGR2), Type II IL receptors; chemokine receptors, such as CC chemokine receptors, CXC chemokine receptors, CX3C chemokine receptors, XC chemokine receptors; tumor necrosis receptor superfamily receptors, such as TNFRSF5/CD40, TNFRSF8/CD30, TNFRSF7/CD27, TNFRSF1A/TNFR1/CD120a, TNFRSF1B/TNFR2/CD120b; TGF-beta receptors, such as TGF-beta receptor 1, TGF-beta receptor 2; Ig superfamily receptors, such as IF-1 receptors, CSF-1R, PDGFR (PDGFRA, PDGFRB), or SCFR.

In some embodiments, the antibody or antigen-binding fragment binds an antigen selected from CD47, CD3, CD19, CD20, CD22, CD30, CD33, CD34, CD40, CD44, CD52, CD70, CD79a, CD123, Her-2, EphA2, lymphocyte associated antigen 1, VEGF or VEGFR, CTLA-4, LIV-1, nectin-4, CD74, SLTRK-6, EGFR, CD73, PD-L1, CD163, CCR4, CD147, EpCam, Trop-2, CD25, C5aR, Ly6D, alpha v integrin, B7H3, B7H4, Her-3, folate receptor alpha, GD-2, CEACAM5, CEACAM6, c-MET, CD266, MUC1, CD10, MSLN, sialyl Tn, Lewis Y, CD63, CD81, CD98, CD166, tissue factor (CD 142), CD55, CD59, CD46, CD164, TGF beta receptor 1 (TGFpR1), TGFpR2, TGFpR3, FasL, MerTk, Axl, Clecl2A, CD352, FAP, CXCR3, and CD5.

### Non-Cleavable Linker (L)

In some embodiments, the linker is a non-cleavable linker. Example of a non-cleavable linker is stable under physiological conditions and at a diseased site such as a tumor site or an inflammatory disease site. In some embodiments, the non-cleavable linker is rich in amino acid residues G and S. In some embodiments, the non-cleavable linker includes a “G4S” repeat. In some embodiments, the non-cleavable linker is a polypeptide chain comprising at least 3 residues. Portions of such linkers may be flexible, hydrophilic, and have little or no secondary structure of their own (linker portions or flexible linker portions). Linkers of at least 3 amino acids may be used to join domains and/or regions that are positioned near to one another after the molecule has assembled. Longer linkers may also be used. In some embodiments, linkers may be about any one of: 1, 2, 3, 4, 5, 6, 7, 8, 9, 10, 11, 12, 13, 14, 15, 16, 17, 18, 19, 20, 25, 30, 35, 40, 45, 50, 100, 125, 150, 175 or 200 residues. When multiple linkers are used to interconnect portions of the molecule, the linkers may be the same or different (e.g., the same or different length and/or amino acid sequence).

In some embodiments, the non-cleavable linker comprises or consists of a Gly-Ser linker. As used herein, the term “Gly-Ser linker” refers to a peptide that consists of glycine and serine residues. In some embodiments, an exemplary Gly-Ser linker comprises an amino acid sequence of GSG (SEQ ID NO: 17). In some embodiments, an exemplary Gly-Ser linker comprises an amino acid sequence of the formula (Gly₄Ser)ₙ, wherein n is a positive integer (e.g., 1, 2, 3, 4, 5, 6, 7, 8, 9, or 10). In some embodiments, a preferred Gly-Ser linker is (Gly₄Ser)₁. In some embodiments, a preferred Gly-Ser linker is (Gly₄Ser)₂. In some embodiments, a preferred Gly-Ser linker is (Gly₄Ser)₃. In some embodiments, a preferred Gly-Ser linker is (Gly₄Ser)₄. In some embodiments, a preferred Gly-Ser linker is (Gly₄Ser)₅. In yet other aspects, two or more Gly-Ser linkers are incorporated in series in a polypeptide linker.

In some embodiments, a non-cleavable linker used in the prodrug described herein comprises an immunoglobulin (Ig)/antibody hinge region. In one embodiment, the hinge region is obtained from an IgG1 antibody. In one embodiment, the term Ig “hinge” region refers to a polypeptide comprising an amino acid sequence that shares sequence identity, or similarity, with a portion of a naturally-occurring Ig hinge region sequence, which includes the cysteine residues at which the disulfide bonds link the two heavy chains of the immunoglobulin.

In some embodiments, a non-cleavable linker is used to link any of the components of a prodrug provided herein.

In some embodiments, the non-cleavable linker comprises the amino acid sequence of any one of SEQ ID NOs: 17-21 or a variant thereof having at least 80% (such as at least about any one of 80%, 85%, 90%, 95%, 96%, 97%, 98%, or 99%) sequence identity to the amino acid sequence of any one of SEQ ID NOs: 17-21.

### Half-Life Extension Moiety (C)

Preferably, the prodrug comprises an in vivo half-life extension moiety (C). The term half-life extension moiety refers to a moiety that extends the half-life of the target component in serum. A long half-life in vivo is important for therapeutic molecules, for example, cytokines that are administered to a subject generally have a short half-life since they are normally cleared rapidly from the subject by mechanisms including clearance by the kidney and endocytic degradation. Increasing the in vivo half-life of therapeutic molecules with naturally short half-lives allows for a more acceptable and manageable dosing regimen without sacrificing effectiveness. Thus, in the prodrug provided herein, preferably, a half-life extension moiety is linked to the biologically active moiety for the purpose of extending the half-life in vivo.

As used herein, a “half-life extension moiety” increases the in vivo half-life and improve PK, for example, by altering its size (e.g., to be above the kidney filtration cutoff), shape, hydrodynamic radius, charge, or parameters of absorption, biodistribution, metabolism, and elimination. An exemplary way to improve the PK of a polypeptide is by expression of an element in the polypeptide chain that binds to receptors that are recycled to the plasma membrane of cells rather than degraded in the lysosomes, such as the FcRn receptor on endothelial cells and transferrin receptor. Three types of proteins, e.g., human IgGs, HSA (or fragments), and transferrin, persist for much longer in human serum than would be predicted just by their size, which is a function of their ability to bind to receptors that are recycled rather than degraded in the lysosome. These proteins or fragments of them that retain the FcRn binding are routinely linked to other polypeptides to extend their serum half-life.

In some embodiments, the half-life extension moiety (C) can also be an antibody or antigen-binding fragment that binds to a protein with a long serum half-life such as serum albumin, transferrin, and the like. Examples of such antibodies or antigen-binding fragments thereof include a polyclonal antibody, a recombinant antibody, a human antibody, a humanized antibody a single-chain variable fragment (scFv), a single-domain antibody such as a heavy chain variable domain (V_{H}), a light chain variable domain (V_{L}) and a variable domain of camelid-type nanobody (V_{HH}), a dAb and the like.

In some embodiments, the half-life extension moiety (C) could also be functioned as a linker, optionally as a non-cleavable linker (L).

In some embodiments, the half-life extension moiety is an antibody Fc domain (e.g., a human IgG1, IgG2, IgG3, or IgG4 Fc) or fragment thereof that is capable of FcRn-mediated recycling, such as any heavy chain polypeptide or portion thereof that is capable of FcRn-mediated recycling. In some embodiments, the Fc domain is a monomer. In some embodiments, the Fc domain is a dimer, comprising a first Fc domain and a second Fc domain.

### Fc Domain

In some embodiments, the Fc domain is derived from any of IgA, IgD, IgE, IgG, and IgM, and subtypes thereof. IgG has the highest serum content and longest serum half-life among all immunoglobulins. Unlike other immunoglobulins, IgG is effectively recycled after binding to Fc receptors (FcRs). In some embodiments, the Fc domain is derived from an IgG (e.g., IgG1, IgG2, IgG3, or IgG4). In some embodiments, the Fc domain is derived from a human IgG. In some embodiments, the Fc domain comprises CH2 and CH3 domains. In some embodiments, the Fc domain further comprises full or part of the hinge region. In some embodiments, the Fc domain is derived from a human IgG1 or human IgG4. In some embodiments, the two subunits of the Fc domain dimerize via one or more (e.g., 1, 2, 3, 4, or more) disulfide bonds. In some embodiments, each subunit of the Fc domain comprises a full-length Fc sequence. In some embodiments, each subunit of the Fc domain comprises an N-terminus truncated Fc sequence, such as a truncated Fc domain with fewer N-terminal cysteines in order to reduce disulfide bond mispairing during dimerization. In some embodiments, the Fc domain is truncated at the N-terminus, e.g., lacks the first 1, 2, 3, 4, 5, 6, 7, 8, 9, or 10 amino acids of a complete immunoglobulin Fc domain.

In some embodiments, the Fc domain contains one or more mutations, such as insertion, deletion, and/or substitution.

In some embodiments, the Fc domain contains one or more amino acid mutations altering effector function, the Fc domain is engineered (e.g., comprises one or more amino acid mutations) to have altered binding to an FcR, specifically altered binding to an Fcγ receptor (responsible for ADCC), and/or altered effector function, such as altered antibody-dependent cell-mediated cytotoxicity (ADCC), Antibody-Dependent Cellular Phagocytosis (ADCP), and/or Complement-Dependent Cytotoxicity (CDC). Preferably, such amino acid mutation(s) does not reduce binding to FcRn receptors (responsible for half-life).

Fc domain (e.g., human IgG1 Fc) mutated to remove one or more effector functions such as ADCC, ADCP, or CDC, is hereinafter referred to as “effectless” or “almost effectless” Fc. For example, in some embodiments, the Fc is an effectless human IgG1 Fc comprising one or more of the following mutations (such as in each of Fc subunits): L234A, L235E, G237A, A330S, and P331S. In some embodiments, the Fc domain comprises L234A and L235A (“LALA”) mutations. The combinations of K322A, L234A, and L235A in IgG1 Fc are sufficient to almost completely abolish FcγR and C1q binding (Hezareh et al. J Virol 75, 12161-12168, 2001). MedImmune identified that a set of three mutations L234F/L235E/P331S have a very similar effect (Oganesyan et al., Acta Crystallographica 64, 700-704, 2008). In some embodiments, the Fc moiety comprises a modification of the glycosylation on N297 of the IgG1 Fc domain, which is known to be required for optimal FcR interaction. The Fc domain modification can be any suitable IgG Fc engineering mentioned in Wang et al. (“IgG Fc engineering to modulate antibody effector functions,” Protein Cell. 2018 January; 9(1): 63-73), the content of which is incorporated herein by reference in its entirety.

### Glycosylation Variants

In some embodiments, the Fc domain is altered to increase or decrease the extent to which the construct is glycosylated. The addition or deletion of glycosylation sites to an Fc domain may be conveniently accomplished by altering the amino acid sequence such that one or more glycosylation sites are created or removed.

Native Fc-containing proteins produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an Fc moiety may be made in order to create certain improved properties.

In some embodiments, the Fc domain described herein is provided having a carbohydrate structure that lacks fucose attached (directly or indirectly) to the Fc domain. For example, the amount of fucose in such Fc domain may be from about 1% to about 80%, from about 1% to about 65%, from about 5% to about 65%, or from about 20% to about 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc domain (EU numbering of Fc region residues); however, Asn297 may also be located about ±3 amino acids upstream or downstream of position 297, i.e., between positions 294 and 300, due to minor sequence variations in Fc domains. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated Fc-containing proteins include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

### Effector Function Variants

In some embodiments, the present application contemplates an Fc domain that possesses some but not all Fc effector functions, which makes it a desirable candidate for applications in which the half-life in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the Fc domain lacks FcγR binding (hence likely lacking ADCC activity), but retains FcRn binding ability. The primary cells for mediating ADCC, Natural Killer (NK) cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Ep. Med. 166:1351-1361(1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the Fc domain is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Fc domain with reduced effector function includes those with substitution of one or more of Fc region residues 238, 265, 269, 270, 297, 327, and 329 (U.S. Pat. No. 6,737,056). Such Fc mutants include substitutions at two or more of amino acid positions 265, 269, 270, 297, and 327, including the so-called “DANA” Fc mutant with substitution of residues 265 and 297 to alanine (U.S. Pat. No. 7,332,581). Certain antibody variants with improved or diminished binding to FcRs are described (see, e.g., U.S. Pat. No. 6,737,056; WO 2004/056312, and Shields et al., J. Biol. Chem. 9(2): 6591-6604 (2001)). In some embodiments, alterations are made in the Fc domain that results in altered (i.e., either improved or diminished) C1q binding and/or CDC, e.g., as described in U.S. Pat. No. 6,194,551, WO 99/51642, and Idusogie et al. J. Immunol. 164: 4178-4184 (2000).

In some embodiments, the Fc domain comprises one or more amino acid substitutions, which increase the half-life and/or improve binding to the neonatal Fc receptor (FcRn). Antibodies with increased half-lives and improved binding to the neonatal FcRn, which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc domain with one or more substitutions therein which improve binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc domain variants.

### Cysteine-Engineered Variants

In some embodiments, it may be desirable to create a cysteine-engineered Fc domain, in which one or more residues of an Fc domain are substituted with cysteine residues. In some embodiments, the substituted residues occur at accessible sites of the Fc domain. By substituting those residues with cysteine, reactive thiol groups are thereby positioned at accessible sites of the Fc domain and may be used to conjugate the molecule to other moieties, such as drug moieties or linker-drug moieties, to create a long-acting drug or prodrug conjugate. In some embodiments, any one or more of the following residues may be substituted with cysteine: A118 (EU numbering) of the heavy chain; and S400 (EU numbering) of the heavy chain Fc domain. Cysteine engineered molecules may be generated as described, e.g., in U.S. Pat. No. 7,521,541.

In some embodiments, the Fc domain is derived from an IgG1 Fc. In some embodiments, the Fc domain is derived from a human IgG1 Fc. In some embodiments, the Fc moiety is a wildtype IgG1 Fc (IGHG1*05). In some embodiments, the Fc domain is a natural variant of IgG1 (e.g., IGHG1*03, which comprises D239E and L241M double mutations relative to IGHG1*05). In some embodiments, the Fc domain does not comprise the hinge region of an IgG1 Fc. In some embodiments, the Fc domain comprises at most about 5 amino acids truncated from the N-terminus of an IgG1 Fc, such as truncating the first, the first two, the first three, the first four, or the first five amino acids from the N-terminus of the IgG1 Fc. In some embodiments, the Fc domain comprises one or more ineffective mutations and/or deglycosylation mutation(s).

In some embodiments, the Fc domain is derived from an IgG4 Fc. In some embodiments, the Fc domain is derived from a human IgG4 Fc. In some embodiments, the Fc domain is a wildtype IgG4 Fc. In some embodiments, the Fc domain is a natural variant of IgG4. In some embodiments, the Fc domain does not comprise the hinge region of an IgG4 Fc. In some embodiments, the Fc domain comprises at most about 5 amino acids truncated from the N-terminus of an IgG4 Fc, such as truncating the first, the first two, the first three, the first four, or the first five amino acids from the N-terminus of the IgG4 Fc. In some embodiments, the Fc domain comprises one or more ineffective mutations and/or deglycosylation mutation(s).

Strategies of forming heterodimers of Fc-fusion polypeptides or bispecific antibodies are well known (see, e.g., Spies et al., Mol Imm. (2015) 67(2)(A): 95-106). For example, in some embodiments, the first and/or second polypeptide chain of Fc domain each contain one or more modifications that promote heterodimerization of the first and the second Fc domain. As such, one or more amino acid modifications can be made to the first Fc domain and one or more amino acid modifications can be made to the second Fc domain using any strategy available in the art, including any strategy as described in Klein et al. (2012), MAbs, 4(6): 653-663. Exemplary strategies and modifications are the “knob into holes” approach. In some embodiments, the first Fc domain comprising a CH3 domain is a heavy chain polypeptide or a fragment thereof. The CH3 domains of the two Fc domains can be altered by the “knobs-into-holes” technology (Fc knob and Fc hole), which is described in detail with several examples in, e.g., WO 1996/027011; Ridgway, J. B. et al. Protein Eng (1996) 9(7): 617-621; Merchant, A. M., et al, Nat. Biotechnoi. (1998) 16(7): 677-681. See also Klein et al. (2012), MAbs, 4(6): 653-663. Using the knob-into-holes approach, the interaction surfaces of the two CH3 domains are altered to increase the heterodimerization of the two moieties containing the two altered CH3 domains. This occurs by introducing a bulky residue into the CH3 domain of one of the Fc domains, which acts as the “knob.” Then, in order to accommodate the bulky residue, a “hole” is formed in the other Fc domain that can accommodate the knob. Either of the altered CH3 domains can be the “knob” while the other can be the “hole.” The introduction of a disulfide bridge further stabilizes the heterodimers (Merchant, A. M., et al, Nat. Biotechnoi (1998) 16(7); Atwell, S., et al, J. Mol, Biol. (1997) 270(1): 26-35) as well as increases yield. It is known that heterodimerization can be achieved by introducing the T366W and/or S354C mutations in a heavy chain to create the “knob” and by introducing the T366S, L368A, Y407V and/or Y349C mutations in a heavy chain to create the “hole” (numbering of the residues according to the Kabat EU numbering system). Carter et al. (2001), J. Immunol. Methods, 248: 7-15; Klein et al. (2012), MAbs, 4(6): 653-663.

In some embodiments, the Fc domain or fragment thereof comprises the mutations of T366S, L368A, and Y407V to form a ‘hole’. In some embodiments, the Fc domain or fragment thereof comprises the mutation of T366W to form a ‘knob’. In some embodiments, the Fc domain or fragment thereof includes the mutations Y349C, T366S, L368A, and Y407V to form a ‘hole’. In some embodiments, the Fc domain or fragment thereof includes the mutations S354C and T366W to form a ‘knob’. In some embodiments, the first Fc domain or fragment thereof includes the hole mutations, and the second Fc domain or fragment thereof includes the knob mutation. In some embodiments, the first Fc domain or fragment thereof includes the knob mutations, and the second Fc domain or fragment thereof includes the hole mutation, numbering of the residues according to the EU numbering system.

In some embodiments, the knobs-into-holes mutation are present in the Fc domains in addition to the LALA (L234A and L235A) mutation.

In some embodiments, the first Fc domain comprises the amino acid sequence of SEQ ID NO: 27 or a variant thereof having at least 80% (such as at least about any one of 80%, 85%, 90%, 95%, 96%, 97%, 98%, or 99%) sequence identity to the amino acid sequence of SEQ ID NO: 27, and the second Fc domain comprises the amino acid sequence of SEQ ID NO: 28 or a variant thereof having at least 80% (such as at least about any one of 80%, 85%, 90%, 95%, 96%, 97%, 98%, or 99%) sequence identity to the amino acid sequence of SEQ ID NO: 28.

In some embodiments, the first Fc domain comprises the amino acid sequence of SEQ ID NO: 28 or a variant thereof having at least 80% (such as at least about any one of 80%, 85%, 90%, 95%, 96%, 97%, 98%, or 99%) sequence identity to the amino acid sequence of SEQ ID NO: 28, and the second Fc domain comprises the amino acid sequence of SEQ ID NO: 27 or a variant thereof having at least 80% (such as at least about any one of 80%, 85%, 90%, 95%, 96%, 97%, 98%, or 99%) sequence identity to the amino acid sequence of SEQ ID NO: 27.

In some embodiments, the first Fc domain comprises the amino acid sequence of SEQ ID NO: 29 or a variant thereof having at least 80% (such as at least about any one of 80%, 85%, 90%, 95%, 96%, 97%, 98%, or 99%) sequence identity to the amino acid sequence of SEQ ID NO: 29, and the second Fc domain comprises the amino acid sequence of SEQ ID NO: 30 or a variant thereof having at least 80% (such as at least about any one of 80%, 85%, 90%, 95%, 96%, 97%, 98%, or 99%) sequence identity to the amino acid sequence of SEQ ID NO: 30.

In some embodiments, the first Fc domain comprises the amino acid sequence of SEQ ID NO: 30 or a variant thereof having at least 80% (such as at least about any one of 80%, 85%, 90%, 95%, 96%, 97%, 98%, or 99%) sequence identity to the amino acid sequence of SEQ ID NO: 30, and the second Fc domain comprises the amino acid sequence of SEQ ID NO: 29 or a variant thereof having at least 80% (such as at least about any one of 80%, 85%, 90%, 95%, 96%, 97%, 98%, or 99%) sequence identity to the amino acid sequence of SEQ ID NO: 29.

### Prodrugs

One aspect of the present application provides activatable prodrugs that are metabolized in vivo to become active therapeutics and have fewer side effects and better target specificity. In some embodiments, the prodrugs comprise one or more biologically active moiety (B), one or more cleavable moiety (CM), and one or more masking polypeptide (MP). In some embodiments, the masking polypeptide (MP) in the prodrugs inhibits the biological functions of the biologically active moiety (B). The prodrugs may be activated at a target site (e.g., at a tumor site or the surrounding environment) in the patient by protease digestion of the cleavable moiety (CM) and the consequent release of the masking polypeptide (MP) from the prodrug, exposing the previously masked biologically active moiety (B), and allowing the biologically active moiety (B) to exert its biological functions on the target cell.

In some embodiments, the prodrugs further comprise one or more non-cleavable linker (L).

In some embodiments, the prodrug provided herein, wherein the masking polypeptide (MP) and the biologically active moiety (B) are linked through the cleavable moiety (CM).

In some embodiments, the prodrug provided herein, wherein the cleavable moiety (CM) is linked to the masking polypeptide (MP), preferably through a non-cleavable linker (L).

In some embodiments, the prodrug provided herein, wherein the cleavable moiety (CM) is linked to the biologically active moiety (B), preferably through a non-cleavable linker.

In some embodiments, the prodrugs further comprise one or more half-life extension moiety (C).

In some embodiments, the prodrug provided herein, wherein the biologically active moiety (B) is linked to the half-life extension moiety (C).

In some embodiments, the prodrug provided herein, wherein the biologically active moiety (B) is linked to the half-life extension moiety (C), preferably through a non-cleavable linker (L).

In some embodiments, the prodrug provided herein, wherein the masking polypeptide (MP) is linked to the half-life extension moiety (C) through the cleavable moiety (CM).

In some embodiments, the prodrug provided herein, wherein the cleavable moiety (CM) is linked to the half-life extension moiety (C), preferably through a non-cleavable linker (L).

In some embodiments, the prodrug provided herein, wherein the cleavable moiety (CM) is linked to the masking polypeptide (MP), preferably through a non-cleavable linker (L).

In some embodiments, the prodrug provided herein, wherein the prodrug comprises the construct in an N to C or in a C to N-terminal direction: B-C-CM-MP, wherein the “-” represents covalent bond with or without non-cleavable linker (L).

In some embodiments, the prodrug provided herein, wherein the prodrug comprises the construct in an N to C or in a C to N-terminal direction: C-B-CM-MP, wherein the “-” represents covalent bond with or without non-cleavable linker (L).

In some embodiments, the prodrug provided herein is a monomer. In some embodiments, the prodrug provided herein is a dimer. In some embodiments, the dimer is monovalent. In some embodiments, the dimer is bivalent. In some embodiments, the dimer is a homodimer. In some embodiments, the dimer is a heterodimer.

In some embodiments, the prodrug provided herein is a dimer, wherein one monomer comprises the construct in an N to C or in a C to N-terminal direction: C-B, the other monomer comprises the construct in an N to C or in a C to N-terminal direction: C-CM-MP, wherein the “-” represents covalent bond with or without non-cleavable linker (L).

### Example of Antibody Prodrug

The present application provides antibody prodrug with masking polypeptide (MP) and cleavable moiety (CM), which is metabolized in vivo to become active antibody, the masked antibody has fewer side effects. In some embodiments, the masking polypeptide (MP) is linked to the N-terminus or C-terminus of the V_{H} domain through the cleavable moiety (CM). In some embodiments, the masking polypeptide (MP) is linked to the N-terminus or C-terminus of the V_{L} domain through the cleavable moiety (CM). In some embodiments, the masking polypeptide (MP) is linked to N-terminus of the V_{H} domain and the N-terminus of the V_{L} domain through the cleavable moiety (CM).

In some embodiments, the antibody prodrug is a TNFR2 antibody prodrug. The illustrative structure of the anti-TNFR2 antibody or the antibody prodrugs are shown in FIGS. 15A-15D.

In some embodiments, an anti-TNFR2 antibody prodrug provided herein comprises (i) an antibody or antigen-binding fragment thereof that specifically binds to human TNFR2, (ii) one or more masking polypeptide (MP) of the disclosure and (iii) one or more cleavable moiety (CM) of the disclosure.

In some embodiments, the masking polypeptide (MP) reduces the binding affinity of the anti-TNFR2 antibody or antigen-binding fragment to human TNFR2 compared to the anti-TNFR2 antibody or antigen-binding fragment without the masking polypeptide (MP). In some embodiments, the binding affinity is reduced by at least about 10-fold compared to an antibody or antigen-binding fragment thereof without the masking polypeptide (MP). In some embodiments, the binding affinity is reduced by at least about 100-fold compared to an antibody or antigen-binding fragment thereof without the masking polypeptide (MP). In some embodiments, the binding affinity is reduced by between about 200-fold and about 1500-fold compared to an antibody or antigen-binding fragment thereof without the masking polypeptide (MP). In some embodiments, the masking polypeptide (MP) comprises the amino acid sequence of any one of SEQ ID NOs: 1-5 or a variant thereof having at least about 90% (such as at least about any one of 91%, 92%, 93%, 94%, 95%, 96%, 97%, 98%, or 99%) sequence identity to the amino acid sequence of any one of SEQ ID NOs: 1-5.

In some embodiments, the anti-TNFR2 antibody prodrug provided herein, wherein, the masking polypeptide (MP) is linked to the N-terminus and/or C-terminus of the V_{H} domain through the cleavable moiety (CM).

In some embodiments, the anti-TNFR2 antibody prodrug provided herein, wherein, the masking polypeptide (MP) is linked to the N-terminus and/or C-terminus of the V_{L} domain through the cleavable moiety (CM).

In some embodiments, the anti-TNFR2 antibody prodrug provided herein, wherein, the masking polypeptide (MP) is linked to the N-terminus and/or C-terminus of the V_{H} domain through the cleavable moiety (CM); and the masking polypeptide (MP) is linked to the N-terminus and/or C-terminus of the V_{L} domain through the cleavable moiety (CM).

In some embodiments, the anti-TNFR2 antibody prodrug, wherein the anti-TNFR2 antibody is a full-length antibody. In some embodiments, the isolated anti-TNFR2 antibody is a full-length IgG1, IgG2, IgG3, or IgG4 antibody.

In some embodiments, the IgG1 is human IgG1. In some embodiments, the IgG1 is human wild-type human IgG1. In some embodiments, the IgG1 contains one or more mutations relative to human wild-type human IgG1.

In some embodiments, the IgG2 is human IgG2. In some embodiments, the IgG2 is human wild-type human IgG2. In some embodiments, the IgG2 contains one or more mutations relative to human wild-type human IgG2.

In some embodiments, the IgG3 is human IgG3. In some embodiments, the IgG3 is human wild-type human IgG3. In some embodiments, the IgG3 contains one or more mutations relative to human wild-type human IgG3.

In some embodiments, the IgG4 is human IgG4. In some embodiments, the IgG4 is human wild-type human IgG4. In some embodiments, the IgG4 contains one or more mutations relative to human wild-type human IgG4.

In some embodiments, the anti-TNFR2 antibody prodrug comprises two heavy chains and two light chains, wherein the masking polypeptide (MP) is linked to the N-terminus of one or two of the heavy chains through the cleavable moiety (CM).

In some embodiments, the anti-TNFR2 antibody prodrug comprises two heavy chains and two light chains, wherein the masking polypeptide (MP) is linked to the N-terminus of one or two of the light chains through the cleavable moiety (CM).

In some embodiments, the anti-TNFR2 antibody prodrug comprises two heavy chains and two light chains, wherein the masking polypeptide (MP) is linked to the N-terminus of two of the heavy chains through the cleavable moiety (CM); and the masking polypeptide (MP) is linked to the N-terminus of two of the light chains through the cleavable moiety (CM).

In some embodiments, the cleavable moiety (CM) is linked to the masking polypeptide (MP), preferably, through a non-cleavable linker (L).

In some embodiments, the cleavable moiety (CM) is linked to the V_{H} domain, and/or V_{L} domain, preferably, through a non-cleavable linker (L).

In some embodiments, the cleavable moiety (CM) as used herein comprises cleavage site, that can be cleaved under certain conditions, thereby separating its N-terminal fragment from its C-terminal fragment. By including a cleavage site between the masking polypeptide and the heavy or light chain of the antibody, the masking polypeptide can be removed at the cleavage site under the designed conditions, thereby releasing the fully functional anti-TNFR2 antibody.

In some embodiments, selection of a suitable cleavable moiety (CM) would depend on the desired action site of the anti-TNFR2 antibody. For example, when a tumor site is the desired action site, a cleavage site of a protease specific to the tumor is used for constructing a masked anti-TNFR2 antibody intended to act at the tumor site. A protease specific to a tumor refers to any protease that has an elevated level and/or activity at the tumor site relative to normal tissues.

In some embodiments, the protease cleavage site can be a cleavage site of a matrix metalloproteinase (MMP). In some embodiments, the protease cleavage site can be a cleavage site of MMP2. In some embodiments, the protease cleavage site can be a cleavage site of MMP9. In some embodiments, the protease cleavage site can be a cleavage site of MMP2 and MMP9. Additional information regarding tumor-specific proteases and corresponding cleavage sites is known in the art, for example, disclosed in Vasiljeva et al., Scientific Reports, 10:5894, 2020, the relevant disclosures of which are incorporated by reference for the subject matter and purpose referenced herein. In some embodiments, the cleavable moiety (CM) comprises the amino acid sequence of any one of SEQ ID NOs: 8-16 or a variant thereof having at least about 90% (such as at least about any one of 91%, 92%, 93%, 94%, 95%, 96%, 97%, 98%, or 99%) sequence identity to the amino acid sequence of any one of SEQ ID NOs: 8-16.

In some embodiments, the anti-TNFR2 antibody prodrug provided herein, wherein the anti-TNFR2 antibody comprises a V_{H} comprising an HC-CDR1 comprising the amino acid sequence of SEQ ID NO: 50, an HC-CDR2 comprising the amino acid sequence of SEQ ID NO: 51, and an HC-CDR3 comprising the amino acid sequence of SEQ ID NO: 52, or a variant thereof comprising up to about 5 amino acid substitutions in the HC-CDRs; and a V_{L} comprising an LC-CDR1 comprising the amino acid sequence of SEQ ID NO: 53, an LC-CDR2 comprising the amino acid sequence of SEQ ID NO: 54, and an LC-CDR3 comprising the amino acid sequence of SEQ ID NO: 55, or a variant thereof comprising up to about 5 amino acid substitutions in the LC-CDRs.

In some embodiments, the anti-TNFR2 antibody prodrug provided herein, wherein the anti-TNFR2 antibody comprises a V_{H} comprising the amino acid sequence of SEQ ID NO: 56, or a variant thereof having at least about 80% (such as at least about any one of 80%, 85%, 90%, 95%, 96%, 97%, 98%, or 99%) sequence identity to the amino acid sequence of SEQ ID NO: 56; and a V_{L} comprising the amino acid sequence of SEQ ID NO: 57, or a variant thereof having at least about 80% (such as at least about any one of 80%, 85%, 90%, 95%, 96%, 97%, 98%, or 99%) sequence identity to the amino acid sequence of SEQ ID NO: 57.

In some embodiments, the anti-TNFR2 antibody prodrug comprises the heavy chain comprising the construct: MP-CM-V_{H}-C_{H}1-hinge-C_{H}2-C_{H}3 in an N to C-terminal direction, wherein “-” represents covalent bond with or without non-cleavable linker (L). In some embodiments, the heavy chain of the anti-TNFR2 antibody prodrug comprises the amino acid sequence of SEQ ID NO: 58 or a variant thereof having at least about 80% (such as at least about any one of 80%, 85%, 90%, 95%, 96%, 97%, 98%, or 99%) sequence identity to the amino acid sequence of SEQ ID NO: 58.

In some embodiments, the anti-TNFR2 antibody prodrug comprises the light chain comprising the construct: MP-CM-V_{L}-C_{L} in an N to C-terminal direction, wherein “-” represents covalent bond with or without non-cleavable linker (L). In some embodiments, the light chain of the anti-TNFR2 antibody prodrug comprises the amino acid sequence of SEQ ID NO: 61 or a variant thereof having at least about 80% (such as at least about any one of 80%, 85%, 90%, 95%, 96%, 97%, 98%, or 99%) sequence identity to the amino acid sequence of SEQ ID NO: 61.

In some embodiments, the anti-TNFR2 antibody prodrug comprises two heavy chains and two light chains, wherein both the heavy chains comprise the amino acid sequence of SEQ ID: 58; or a variant thereof having at least 80% (such as at least about any one of 80%, 85%, 90%, 95%, 96%, 97%, 98%, or 99%) sequence identity to the amino acid sequence of SEQ ID: 58; and both the light chains comprise the amino acid sequence of SEQ ID: 59; or a variant thereof having at least 80% (such as at least about any one of 80%, 85%, 90%, 95%, 96%, 97%, 98%, or 99%) sequence identity to the amino acid sequence of SEQ ID: 59.

In some embodiments, the anti-TNFR2 antibody prodrug comprises two heavy chains and two light chains, wherein both the heavy chains comprise the amino acid sequence of SEQ ID: 60; or a variant thereof having at least 80% (such as at least about any one of 80%, 85%, 90%, 95%, 96%, 97%, 98%, or 99%) sequence identity to the amino acid sequence of SEQ ID: 60; and both the light chains comprise the amino acid sequence of SEQ ID: 61; or a variant thereof having at least 80% (such as at least about any one of 80%, 85%, 90%, 95%, 96%, 97%, 98%, or 99%) sequence identity to the amino acid sequence of SEQ ID: 61.

In some embodiments, the anti-TNFR2 antibody prodrug comprises two heavy chains and two light chains, wherein both the heavy chains comprise the amino acid sequence of SEQ ID: 58; or a variant thereof having at least 80% (such as at least about any one of 80%, 85%, 90%, 95%, 96%, 97%, 98%, or 99%) sequence identity to the amino acid sequence of SEQ ID: 58; and both the light chains comprise the amino acid sequence of SEQ ID: 61; or a variant thereof having at least 80% (such as at least about any one of 80%, 85%, 90%, 95%, 96%, 97%, 98%, or 99%) sequence identity to the amino acid sequence of SEQ ID: 61.

### Example of Cytokine Prodrug

The present application provides IL-15 prodrugs as example that are metabolized in vivo to become active IL-15 cytokine. The IL-15 prodrugs have fewer side effects. In some embodiments, the IL-15 prodrugs have better PK profiles in vivo (e.g., longer half-life).

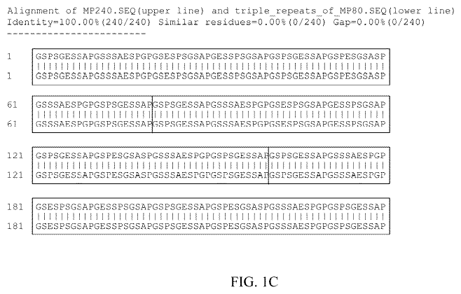

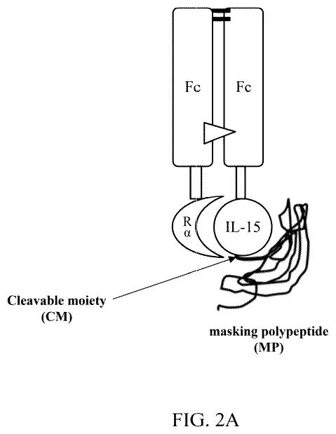

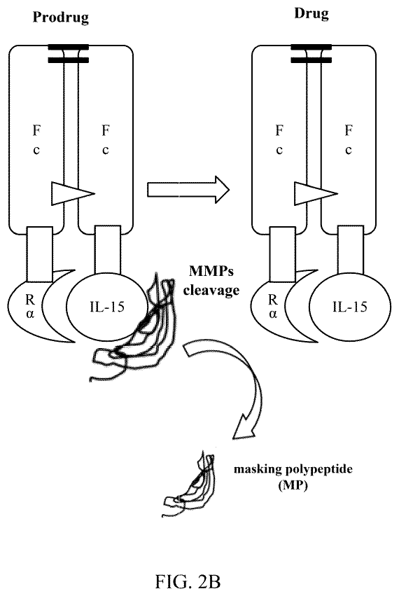

The illustrative structure of the exemplary IL-15 cytokine prodrug is shown in FIG. 2A.

In some embodiments, the IL-15 prodrug provided herein comprises (i) one or more IL-15 cytokine(I), (ii) one or more masking polypeptide (MP) of the disclosure and (iii) one or more cleavable moiety (CM) of the disclosure.

In some embodiments, the IL-15 prodrug provided herein, wherein the IL-15 cytokine (I) and the masking polypeptide (MP) are linked through the cleavable moiety (CM).

In some embodiments, the IL-15 prodrug provided herein, wherein the cleavable moiety (CM) is linked to the IL-15 cytokine, preferably through a non-cleavable linker (L).

In some embodiments, the IL-15 prodrug provided herein, wherein the cleavable moiety (CM) is linked to the masking polypeptide (MP), preferably through a non-cleavable linker (L).

In some embodiments, the IL-15 prodrug provided herein comprises the construct in an N to C-terminal or in a C to N-terminal direction: I-CM-MP, wherein the “-” represents covalent bond with or without non-cleavable linker (L).

In some embodiments, the IL-15 prodrug provided herein further comprise one or more half-life extension moiety (C).

In some embodiments, the IL-15 prodrug provided herein, wherein the IL-15 cytokine (I) is linked to the half-life extension moiety (C).

In some embodiments, the IL-15 prodrug provided herein, wherein the IL-15 cytokine is linked to the half-life extension moiety (C), preferably through a non-cleavable linker (L).

In some embodiments, the IL-15 prodrug provided herein, wherein the masking polypeptide (MP) is linked to the half-life extension moiety (C) through a cleavable moiety (CM).

In some embodiments, the IL-15 prodrug provided herein, wherein the cleavable moiety (CM) is linked to the masking polypeptide (MP) through a non-cleavable linker (L).

In some embodiments, the IL-15 prodrug provided herein, wherein the cleavable moiety (CM) is linked to half-life extension moiety (C), preferably through a non-cleavable linker (L).

In some embodiments, the IL-15 prodrug provided herein comprises the construct in an N to C-terminal or in a C to N-terminal direction: C-I-CM-MP, wherein the “-” represents covalent bond with or without non-cleavable linker (L).

In some embodiments, the IL-15 prodrug provided herein comprises the construct in an N to C-terminal or in a C to N-terminal direction: I-C-CM-MP, wherein the “-” represents covalent bond with or without non-cleavable linker (L)

In some embodiments, the IL-15 prodrug provided herein comprises the construct in an N to C-terminal or in a C to N-terminal direction: C-CM-MP-I, wherein the “-” represents covalent bond with or without non-cleavable linker (L).

In some embodiments, the IL-15 prodrug provided herein is a dimer, wherein one monomer comprises the construct in an N to C-terminal or in a C to N-terminal direction: C-I; and the other monomer comprises the construct in an N to C-terminal or in a C to N-terminal direction: C-CM-MP, and wherein the “-” represents covalent bond with or without non-cleavable linker (L).

In some embodiments, the IL-15 prodrug provided herein comprises (i) one or more IL-15 cytokine (I), (ii) one or more IL-15Rα or a functional fragment thereof (S), (iii) one or more masking polypeptide (MP) of the disclosure and (iv) one or more cleavable moiety (CM) of the disclosure.

In some embodiments, the IL-15 prodrug provided herein, wherein the IL-15 cytokine (I) and the masking polypeptide (MP) are linked through the cleavable moiety (CM).

In some embodiments, the IL-15 prodrug provided herein, wherein the cleavable moiety (CM) is linked to the IL-15 cytokine (I), preferably through a non-cleavable linker (L).

In some embodiments, the IL-15 prodrug provided herein, wherein the cleavable moiety (CM) is linked to the masking polypeptide (MP), preferably through a non-cleavable linker (L).

In some embodiments, the IL-15 prodrug provided herein, wherein the IL-15Rα or a functional fragment thereof (S) is linked to the IL-15 cytokine (I).

In some embodiments, the IL-15 prodrug provided herein, wherein the IL-15Rα or a functional fragment thereof (S) and the masking polypeptide (MP) are linked through the cleavable moiety (CM).

In some embodiments, the IL-15 prodrug provided herein, wherein the cleavable moiety (CM) is linked to the IL-15Rα or a functional fragment thereof (S), preferably through a non-cleavable linker (L).

In some embodiments, the IL-15 prodrug provided herein, wherein the cleavable moiety (CM) is linked to the masking polypeptide (MP), preferably through a non-cleavable linker (L).

In some embodiments, the IL-15 prodrug provided herein, wherein the IL-15Rα or a functional fragment thereof (S) and the IL-15 cytokine (I) are covalently linked. In some embodiments, the IL-15Rα or a functional fragment thereof (S) and the IL-15 cytokine (I) are linked through a non-cleavable linker (L).

In some embodiments, the IL-15 prodrug provided herein, wherein the IL-15Rα or a functional fragment thereof (S) and the IL-15 cytokine (I) are non-covalently linked, and form an IL-15/IL-15Rα complex.

In some embodiment, IL-15 prodrug provided herein, wherein the IL-15 Rα or a functional fragment and IL-15 cytokine are transfected separately and an IL-15/IL-15Rα complex is formed.

In some embodiments, the IL-15 prodrug provided herein further comprises one or more life-extension moiety (C).

In some embodiments, the IL-15 prodrug provided herein, wherein the IL-15 cytokine (I) is linked to the half-life extension moiety (C).

In some embodiments, the IL-15 prodrug provided herein, wherein the IL-15 cytokine (I) is linked to the half-life extension moiety (C), preferably through a non-cleavable linker (L).

In some embodiments, the IL-15 prodrug provided herein, wherein the IL-15Rα or a functional fragment thereof (S) is linked to the half-life extension moiety (C).

In some embodiments, the IL-15 prodrug provided herein, wherein the IL-15Rα or a functional fragment thereof (S) is linked to the half-life extension moiety (C), preferably through a non-cleavable linker (L).

In some embodiments, the IL-15 prodrug provided herein, wherein the masking polypeptide (MP) is linked to the half-life extension moiety (C) through the cleavable moiety (CM).

In some embodiments, the IL-15 prodrug provided herein, wherein the cleavable moiety (CM) is linked to the masking polypeptide (MP), preferably through a non-cleavable linker (L).

In some embodiments, the IL-15 prodrug provided herein, wherein the cleavable moiety (CM) is linked to the half-life extension moiety (C), preferably through a non-cleavable linker (L).

In some embodiments, the IL-15 prodrug provided herein, wherein the IL-15 cytokine (I) is linked to the half-life extension moiety (C), the IL-15Rα or a functional fragment thereof (S) is linked to the IL-15 cytokine (I), and the masking polypeptide (MP) is linked to the IL-15Rα or a functional fragment thereof (S) through the cleavable moiety (CM).

In some embodiments, the IL-15 prodrug provided herein, wherein the IL-15 cytokine (I) is linked to the half-life extension moiety (C), the IL-15Rα or a functional fragment thereof (S) is linked to the half-life extension moiety (C), and the masking polypeptide (MP) is linked to the IL-15 cytokine (I) through the cleavable moiety (CM).

In some embodiments, the IL-15 prodrug provided herein, wherein the IL-15Rα or a functional fragment thereof (S) is linked to the half-life extension moiety (C), the IL-15 cytokine (I) is linked to the IL-15Rα or a functional fragment thereof (S), and the masking polypeptide (MP) is linked to the IL-15 cytokine (I) through the cleavable moiety (CM).

In some embodiments, the IL-15 prodrug provided herein, wherein the IL-15 cytokine (I) is linked to the half-life extension moiety (C), the IL-15Rα or a functional fragment thereof (S) is linked to the half-life extension moiety (C), and the masking polypeptide (MP) is linked to the IL-15Rα or a functional fragment thereof (S) through the cleavable moiety (CM).

In some embodiments, the IL-15 prodrug provided herein, wherein the masking polypeptide (MP) is linked to the half-life extension moiety (C) through the cleavable moiety (CM), the IL-15Rα or a functional fragment thereof (S) is linked to the half-life extension moiety (C), and the IL-15 cytokine (I) is linked to the IL-15Rα or a functional fragment thereof (S).

In some embodiments, the IL-15 prodrug provided herein, wherein the masking polypeptide (MP) is linked to the half-life extension moiety (C) through the cleavable moiety (CM), the IL-15 cytokine (I) is linked to the half-life extension moiety (C), and the IL-15Rα or a functional fragment thereof (S) is linked to the IL-15 cytokine (I).

In some embodiments, the IL-15 prodrug provided herein, wherein the IL-15Rα or a functional fragment thereof (S) and the IL-15 cytokine (I) are covalently linked. In some embodiments, the IL-15Rα or a functional fragment thereof (S) and the IL-15 cytokine (I) are linked through a non-cleavable linker (L).

In some embodiments, the IL-15 prodrug provided herein, wherein the IL-15Rα or a functional fragment thereof (S) and the IL-15 cytokine (I) are non-covalently linked, and form an IL-15/IL-15Rα complex.

In some embodiments, the IL-15 prodrug provided herein comprises the construct in an N to C or in a C to N-terminal direction: C-I-S-CM-MP, wherein the “-” represents covalent bond with or without non-cleavable linker (L).

In some embodiments, the IL-15 prodrug provided herein comprises the construct in an N to C or in a C to N-terminal direction: C-S-I-CM-MP, wherein the “-” represents covalent bond with or without non-cleavable linker (L).

In some embodiments, the IL-15 prodrug provided herein comprises the construct in an N to C or in a C to N-terminal direction: I-C-S-CM-MP, wherein the “-” represents covalent bond with or without non-cleavable linker (L).

In some embodiments, the IL-15 prodrug provided herein comprises the construct in an N to C or in a C to N-terminal direction: S-C-I-CM-MP, wherein the “-” represents covalent bond with or without non-cleavable linker (L).

In some embodiments, the IL-15 prodrug provided herein comprises the construct in an N to C or in a C to N-terminal direction: S-I-C-CM-MP, wherein the “-” represents covalent bond with or without non-cleavable linker (L).

In some embodiments, the IL-15 prodrug provided herein comprises the construct in an N to C or in a C to N-terminal direction: I-S-C-CM-MP, wherein the “-” represents covalent bond with or without non-cleavable linker (L).

In some embodiments, the IL-15 prodrug provided herein is a monomer.

In some embodiments, the IL-15 prodrug provided herein is a dimer. In some embodiments, the dimer is monovalent. In some embodiments, the dimer is bivalent. In some embodiments, the dimer is a homodimer. In some embodiments, the dimer is a heterodimer.

In some embodiments, the IL-15 prodrug provided herein is a dimer, wherein one monomer comprises the first half-life extension moiety (C), the IL-15Rα or a functional fragment thereof (S), the IL-15 cytokine (I); and the other monomer comprises the second half-life extension moiety (C), the masking polypeptide (MP) and the cleavable moiety (CM), wherein the masking polypeptide (MP) is linked to the second half-life extension moiety (C) through the cleavable moiety (CM).

In some embodiments, the IL-15 prodrug provided herein is a dimer, wherein one monomer comprises the first half-life extension moiety (C), the IL-15Rα or a functional fragment thereof (S), the masking polypeptide (MP) and the cleavable moiety (CM); and the other monomer comprises the second half-life extension moiety (C) and the IL-15 cytokine (I).

In some embodiments, the IL-15 prodrug provided herein is a dimer, wherein one monomer comprises the first half-life extension moiety (C), the IL-15 cytokine (I), the masking polypeptide (MP) and a cleavable moiety (CM); and the other monomer comprises the second half-life extension moiety (C) and the IL-15Rα or a functional fragment thereof (S).

In some embodiments, the IL-15 prodrug provided herein is a dimer, wherein one monomer and the other monomer each comprises the half-life extension moiety (C), the IL-15 cytokine (I), the IL-15Rα or a functional fragment thereof (S), the cleavable moiety (CM) and the masking polypeptide (MP).

In some embodiments, the IL-15 prodrug provided herein is a dimer, wherein in one monomer: the IL-15 cytokine (I) is linked to the first half-life extension moiety (C) and the IL-15Rα or a functional fragment thereof (S) is linked to the first half-life extension moiety (C); and in the other monomer: the masking polypeptide (MP) is linked to the second half-life extension moiety (C) through the cleavable moiety (CM).

In some embodiments, the IL-15 prodrug provided herein is a dimer, wherein in one monomer: the IL-15 cytokine (I) is linked to the first half-life extension moiety (C) and the IL-15Rα or a functional fragment thereof (S) is linked to the IL-15 cytokine (I); and in the other monomer: the masking polypeptide (MP) is linked to the second half-life extension moiety (C) through the cleavable moiety (CM).

In some embodiments, the IL-15 prodrug provided herein is a dimer, wherein in one monomer: the IL-15Rα or a functional fragment thereof (S) is linked to the first half-life extension moiety (C) and the masking polypeptide (MP) is linked to the first half-life extension moiety (C) through the cleavable moiety (CM); and in the other monomer: the IL-15 cytokine (I) is linked to the second half-life extension moiety (C).

In some embodiments, the IL-15 prodrug provided herein is a dimer, wherein in one monomer: the IL-15Rα or a functional fragment thereof (S) is linked to the first half-life extension moiety (C) and the masking polypeptide (MP) is linked to the IL-15Rα or a functional fragment thereof (S) through the cleavable moiety (CM); and in the other monomer: the IL-15 cytokine (I) is linked to the second half-life extension moiety (C).

In some embodiments, the IL-15 prodrug provided herein is a dimer, wherein in one monomer: the IL-15 cytokine (I) is linked to the first half-life extension moiety (C) and the masking polypeptide (MP) is linked to the first half-life extension moiety (C) through the cleavable moiety (CM); and in the other monomer: the IL-15Rα or a functional fragment thereof (S) is linked to the second half-life extension moiety (C).

In some embodiments, the IL-15 prodrug provided herein is a dimer, wherein in one monomer: the IL-15 cytokine (I) is linked to the first half-life extension moiety (C) and the masking polypeptide (MP) is linked to the IL-15 cytokine (I) through the cleavable moiety (CM); and in the other monomer: the IL-15Rα or a functional fragment thereof (S) is linked to the second half-life extension moiety (C).

In some embodiments, the IL-15 prodrug provided herein, wherein the IL-15Rα or a functional fragment thereof (S) and the IL-15 cytokine (I) are covalently linked. In some embodiments, the IL-15Rα or a functional fragment thereof (S) and the IL-15 cytokine (I) are linked through a non-cleavable linker (L).

In some embodiments, the IL-15 prodrug provided herein, wherein the IL-15Rα or a functional fragment thereof (S) and the IL-15 cytokine (I) are non-covalently linked, and form an IL-15/IL-15Rα complex.

### IL-15:

Wild-type IL-15 is a member of the four α-helix bundle family with 14-15 kDa molecular weight and 114 amino acids (Fehniger T A, Caligiuri M A. Interleukin 15: biology and relevance to human disease. Blood. 2001; 97:14-32), and is produced by mononuclear phagocytes and other cells of the immune system. IL-15 is essential for natural killer cells (NK), natural killer T cells (NKT), and memory CD8+ T cells development and function.

IL-15 is a cytokine which like IL-2, has originally been described as a T cell growth factor. Both cytokines exert their cell signaling function through binding to a trimeric complex consisting of two shared receptors, the common gamma chain (γc; CD132) and IL-2 receptor beta-chain (IL-2Rβ; CD122), as well as an alpha chain receptor unique to each cytokine: IL-2 receptor alpha (IL-2Rα; CD25) or IL-15 receptor alpha (IL-15Rα; CD215).

IL-15 shares components of the receptor for IL-2, the alpha chain of the IL-2 receptor (IL-2R) is not required, but both beta and common gamma chains are needed for IL-15 mediated bioactivities. (Giri J G, et al. IL-15, a novel T cell growth factor that shares activities and receptor components with IL-2. J Leukoc Biol. 1995 May; 57(5):763-6.). IL-15R consists of three subunits IL-15Rα, IL-2/IL-15Rβ, and γ chain, IL-15Rα is required for high-affinity binding but not signaling by IL-15. IL-15 functions mainly via trans-presentation (TP), during which an APC expressing IL-15 bound to IL-15Rα presents the ligand to the βγ receptor-heterodimer on a neighboring T/NK cell (Kenesei Á, Volkó J, et al. IL-15 Trans-Presentation Is an Autonomous, Antigen-Independent Process. J Immunol. 2021 Nov. 15; 207(10):2489-2500).

As used herein, the term “IL-15 cytokine” or “IL-15” includes wild-type IL-15 or variants thereof, also includes functional fragment thereof. In some embodiments, the IL-15 or IL-15 cytokine of the present application is a wild-type IL-15. In some embodiments, the IL-15 or IL-15 cytokine of the present application is an IL-15 variant. In eukaryotic cells, wild-type IL-15 is synthesized as a precursor polypeptide of 162 amino acids, which is then processed into mature IL-15 by the removal of amino acid residues 1-48. This results in a mature form of IL-15 consisting of 114 amino acids (amino acid residues 49-162) that are secreted in a mature, active form.

In some embodiments, the IL-15 cytokine comprises the amino acid sequence SEQ ID NO: 22 or a variant thereof having at least 90% (such as at least about any one of 91%, 92%, 93%, 94%, 95%, 96%, 97%, 98%, or 99%) sequence identity to the amino acid sequence of SEQ ID NO: 22.

In some embodiments, the IL-15 cytokine comprises the amino acid sequence SEQ ID NO: 23 or a variant thereof having at least 90% (such as at least about any one of 91%, 92%, 93%, 94%, 95%, 96%, 97%, 98%, or 99%) sequence identity to the amino acid sequence of SEQ ID NO: 23.

In some embodiments, the IL-15 cytokine also is an IL-15 variant or functional fragment thereof. In some embodiments, the IL-15 cytokine is any naturally occurring interleukin-15 (IL-15) protein. In some embodiments, the IL-15 cytokine is a variant thereof capable of binding to, or otherwise exhibiting improved or decreased affinity for, an interleukin-15 receptor (IL-15R) or component thereof (e.g., the IL-15Rα, IL-2/IL-15Rβ, and/or γ chain).

In some embodiments, the IL-15 cytokine is an IL-15 variant comprising an amino acid sequence produced by at least one amino acid modification to the amino acid sequence of wild-type IL-15 (e.g., SEQ ID NO: 22). Each at least one amino acid modification can be any amino acid modification, such as a mutation, insertion, or deletion. Any combination of deletion, insertion, and substitution can be made to arrive at the final construct, provided that the final construct possesses the desired characteristics, e.g., retained/improved/decreased ligand-receptor binding, retained/enhanced/reduced bioactivity, etc. In some embodiments, the IL-15 cytokine comprises an amino acid sequence produced by at least 1, at least 2, at least 3, at least 4, at least 5, at least 6, at least 7, at least 8, at least 9, or at least 10 amino acid substitutions in the amino acid sequence of SEQ ID NO: 22.

In some embodiments, the IL-15 cytokine is an IL-15 variant that is well-known in the art, which comprises a sequence that is derived from the human mature wild type IL-15 by at least one mutations, including substitution, deletion or addition, at the residue 45, 48, 51, 52, 64, 67, 68 and/or 72 (see U.S. Pat. No. 9,493,533 B2, and Han K P, et al., IL-15:IL-15 receptor alpha superagonist complex: high-level co-expression in recombinant mammalian cells, purification and characterization. Cytokine. 2011 December; 56(3):804-10). In some embodiments, the IL-15 variant comprise one or more amino acid mutations selected from the group consisting of L45D, L45E, Q48K, S51D, L52D, E64K, I67D, 167E, I68D and N72D, herein the residues are numbering is corresponding to the human mature wild type IL-15 (e.g., SEQ ID NO: 22).

In some embodiments, the IL-15 cytokine comprises any one of the amino acid sequence of SEQ ID NOs: 67-76.

### IL-15Rα or a Functional Fragment Thereof:

The IL-15Rα or a functional fragment thereof according to the present application can be any species of IL-15Rα or a functional fragment thereof.

In some embodiments, the IL-15Rα or a functional fragment thereof is selected from an extracellular region of human IL-15Rα or a sushi domain or functional analogs.

### Extracellular Region of IL-15Rα:

The extracellular region of IL-15Rα is usually defined as the region of an IL-15Rα sequence that extends from its first N-terminal amino acid, to the last amino acid of the tail region (or region rich in glycosylation sites). The tail region of an IL-15Rα sequence can be determined by the skilled person, e.g., through the help of software.

### IL-15Rα_Sushi Domain:

The extracellular region of IL-15Rα contains a domain, which is known as the sushi domain (Wei et al. 2001, J. Immunol. 167:277-282). The IL-15Rα_sushi domain has a beta sheet conformation.

The IL-15Rα_sushi domain, bears most of the binding affinity for IL-15, and behaves as a potent IL-15 agonist by enhancing its binding and biological effects (proliferation and protection from apoptosis) through the IL-15Rβγ heterodimer, whereas it does not affect IL-15 binding and function (Mortier E, et al. J Biol Chem. 2006 Jan. 20; 281(3):1612-9).

It is coded by exon 2 of IL-15Rα (Anderson D M, et al. Functional characterization of the human interleukin-15 receptor alpha chain and close linkage of IL15RA and IL2RA genes. J Biol Chem. 1995 Dec. 15; 270(50):29862-9). It begins at the first exon 2 encoded cysteine residue (C1), and ends at the fourth exon 2 encoded cysteine residue (C4). When considering the IL-15Rα protein sequence in the standard N-terminal to C-terminal orientation, the sushi domain of IL-15Rα can be defined as beginning at the first cysteine residue (C1) after the signal peptide, and ending at the fourth cysteine residue (C4) after the signal peptide. Residues C1 and C4 are both included in the sushi sequence. The IL-15Rα sushi domain can also be determined by analysis of the amino-acid sequence of IL-15Rα with appropriate software such as: Prosite (http://us.expasy.org/prosite/), (http://www.ebi.ac.uk/InterProScan/), SMART (http://elm.eu.org/).

In some embodiments, the IL-15Rα_sushi domain comprises the amino acid sequence of SEQ ID NO: 24 or a variant thereof having at least about 80% (such as at least about any one of 80%, 85%, 90%, 95%, 96%, 97%, 98%, or 99%) sequence identity to the SEQ ID NO: 24.

In some embodiments, the IL-15Rα_sushi domain comprises the amino acid sequence of SEQ ID NO: 25 or a variant thereof having at least about 80% (such as at least about any one of 80%, 85%, 90%, 95%, 96%, 97%, 98%, or 99%) sequence identity to the SEQ ID NO: 25.

In some embodiments, the IL-15Rα_sushi domain comprises the amino acid sequence of SEQ ID NO: 26 or a variant thereof having at least about 80% (such as at least about any one of 80%, 85%, 90%, 95%, 96%, 97%, 98%, or 99%) sequence identity to the SEQ ID NO: 26.

In some embodiments, the IL-15 prodrug, wherein the IL-15 cytokine or the IL-15Rα or a functional fragment thereof (e.g., IL-15Rα_sushi domain) has one or more conservative amino acid substitutions.

“Conservative substitution” refers to the substitution of another amino acid with the same net charge and approximately the same size and shape as the substituted amino acid. When the total number of carbon atoms and heteroatoms on their side chains differ by no more than 4, amino acids with aliphatic or substituted aliphatic amino acid side chains are roughly the same size. When the number of branches on their side chains does not differ by more than one, amino acids have roughly the same shape. Amino acids having a phenyl or substituted phenyl group on the side chain can be considered to be approximately the same in size and shape. Unless otherwise specified, natural amino acids are preferably used for conservative substitutions.

“Amino acid” is used herein in its broadest sense, including both naturally occurring amino acids and non-naturally occurring amino acids, including amino acid analogs and derivatives. The latter includes molecules that contain amino acid moieties. Those skilled in the art will realize that according to this broad definition, amino acids herein include, for example, naturally occurring L-amino acids that form proteins; D-amino acids; chemically modified amino acids, such as amino acid analogs and derivatives; naturally occurring amino acids that do not form protein, such as norleucine, p-alanine, ornithine, GABA, etc.; and chemically synthesized compounds with amino acid characteristics known in the art. The term “protein-forming” as used herein refers to amino acids that can be incorporated into peptides, polypeptides, or proteins of cells through metabolic pathways.

Insertion of non-naturally occurring amino acids, including synthetic non-natural amino acids, substituted amino acids, or one or more D-amino acids, into the peptides (e.g., the IL-15 cytokine in the IL-15 prodrug as described herein) can have multiple benefits. D-amino acid-containing peptides and the like exhibit increased stability in vitro or in vivo compared to their counterparts containing L-amino acid. Therefore, when greater intracellular stability is desired, the construction of peptides, such as by incorporation of D-amino acids, is particularly useful. Particularly, D-peptide and the like are resistant to endogenous peptidase and protease activity, thereby improving the bioavailability of the molecule and extending the lifespan in vivo when needed. In addition, D-peptide and the like cannot be effectively processed for limited presentation by type II major histocompatibility complexes (MHC) to T helper cells, so less prone to induce humoral immune responses in the subject.

Conservative substitutions are shown in Table A. More substantial changes are provided in Table A under the heading of “exemplary substitutions,” and as further described below in reference to amino acid side chain classes. Amino acids may be grouped according to common side-chain properties: (1)hydrophobic: Norleucine, Met, Ala, Val, Leu, Ile; (2) neutral hydrophilic: Cys, Ser, Thr, Asn, Gin; (3) acidic: Asp, Glu; (4) basic: His, Lys, g; (5) residues that influence chain orientation: Gly, Pro; and (6) aromatic: Trp, Tyr, Phe. Non-conservative substitutions will entail exchanging a member of one of these classes for another class. Amino acid substitutions may be introduced into the protein constructs and the products screened for a desired activity mentioned above.

TABLE A
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, | Gln |
| | Lys; Arg | |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; | Tyr |
| | Ala; Tyr | |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

In some embodiments, the IL-15 prodrug provided herein, wherein the masking polypeptide (MP) reduces the binding affinity of the IL-15 or functional fragment thereof to IL-2/IL-15Rβγ compared to IL-15 or functional fragment thereof without the MP. In some embodiments, the binding affinity is reduced by at least about 10-fold compared to IL-15 or functional fragment thereof without the MP. In some embodiments, the binding affinity is reduced by at least about 100-fold compared to IL-15 or functional fragment thereof without the MP. In some embodiments, the binding affinity is reduced by between about 200-fold and about 1500-fold compared to IL-15 or functional fragment thereof without the MP. In some embodiments, the EC₅₀ value increased by at least 5 times, at least 10 times, at least 20 times, at least 50 times, or at least 100 times. In some embodiments, the MP comprises the amino acid sequence of any one of SEQ ID NOs: 1-5 or a variant thereof having at least about 90% (such as at least about any one of 91%, 92%, 93%, 94%, 95%, 96%, 97%, 98%, or 99%) sequence identity to the amino acid sequence of any one of SEQ ID NOs: 1-5.

In some embodiments, the cleavable moiety (CM) comprising a protease cleavage site can be located between the masking polypeptide (MP) and the IL-15 cytokine. The cleavable moiety (CM) as used herein can be cleaved under certain conditions, thereby separating its N-terminal fragment from its C-terminal fragment. By introducing the cleavable moiety (CM), once the masking polypeptide (MP) gets cleaved off from the IL-15 prodrug at the cleavage site under the designed conditions, thereby releasing the fully functional IL-15 cytokine.

In some embodiments, the selection of a suitable cleavable moiety would depend on the desired action site of the IL-15 cytokine. For example, when a tumor site is the desired action site, a cleavage site of a protease specific to the tumor is used for constructing an IL-15 prodrug intended to act at the tumor site. A protease specific to a tumor refers to any protease that has an elevated level and/or activity at the tumor site relative to normal tissues.

In some embodiments, the protease cleavage site can be a cleavage site of a matrix metalloproteinase (MMP). In some embodiments, the protease cleavage site can be a cleavage site of MMP2. In some embodiments, the protease cleavage site can be a cleavage site of MMP9. In some embodiments, the protease cleavage site can be a cleavage site of MMP2 and MMP9. Additional information regarding tumor-specific proteases and corresponding cleavage sites is known in the art, for example, disclosed in Vasiljeva et al., Scientific Reports, 10:5894, 2020, the relevant disclosures of which are incorporated by reference for the subject matter and purpose referenced herein. In some embodiments, the cleavable moiety (CM) comprises the amino acid sequence of any one of SEQ ID NOs: 8-16 or a variant thereof having at least about 90% (such as at least about any one of 91%, 92%, 93%, 94%, 95%, 96%, 97%, 98%, or 99%) sequence identity to the amino acid sequence of any one of SEQ ID NOs: 8-16.

In some embodiments, the IL-15 prodrug provided herein, wherein the half-life extension moiety (C) comprises an Fc domain; preferably, the Fc domain is selected from the group consisting of a human IgG1 Fc domain, a human IgG2 Fc domain, a human IgG3 Fc domain, a human IgG4 Fc domain, an IgA Fc domain, an IgD Fc domain, an IgE Fc domain, and an IgM Fc domain; more preferably, the Fc domain is a human IgG1 Fc domain.

In some embodiments, the IL-15 prodrug provided herein, wherein the half-life extension moiety (C) comprises a human IgG1 Fc domain having L234A and L235A (LALA) mutations, according to EU Numbering system.

In some embodiments, the IL-15 prodrug provided herein, wherein the half-life extension moiety (C) further comprises knobs-into-holes mutations (Fc knob and Fc hole).

In some embodiments, the IL-15 prodrug provided herein, wherein the Fc knob comprises a T366W mutation in the Fc domain, and the Fc hole comprises T366S, L368A, and Y407V mutations in the Fc domain, according to EU Numbering system.

In some embodiments, the IL-15 prodrug provided herein, wherein the Fc knob further comprises S354C mutation, and the Fc hole further comprises Y349C mutation, according to EU Numbering system.

In some embodiments, the IL-15 prodrug provided herein, wherein the Fc knob comprises the amino acid sequence of SEQ ID NO: 28 or a variant thereof having at least about 90% (such as at least about any one of 91%, 92%, 93%, 94%, 95%, 96%, 97%, 98%, or 99%) sequence identity to the amino acid sequence of SEQ ID NO: 28.

In some embodiments, the IL-15 prodrug provided herein, wherein the Fc hole comprises the amino acid sequence of SEQ ID NO: 27 or a variant thereof having at least about 90% (such as at least about any one of 91%, 92%, 93%, 94%, 95%, 96%, 97%, 98%, or 99%) sequence identity to the amino acid sequence of SEQ ID NO: 27.

In some embodiments, the IL-15 prodrug provided herein, wherein the Fc knob and Fc hole further comprises LALA mutation.

In some embodiments, the IL-15 prodrug provided herein, wherein the Fc knob LALA comprises the amino acid sequence of SEQ ID NO: 30 or a variant thereof having at least about 90% (such as at least about any one of 91%, 92%, 93%, 94%, 95%, 96%, 97%, 98%, or 99%) sequence identity to the amino acid sequence of SEQ ID NO: 30.

In some embodiments, the IL-15 prodrug provided herein, wherein the Fc hole LALA comprises the amino acid sequence of SEQ ID NO: 29 or a variant thereof having at least about 90% (such as at least about any one of 91%, 92%, 93%, 94%, 95%, 96%, 97%, 98%, or 99%) sequence identity to the amino acid sequence of SEQ ID NO: 29.

In some embodiments, the IL-15 prodrug provided herein comprises two monomers, and wherein in one monomer, the IL-15Rα or a functional fragment thereof is linked to the Fc domain, and in the other monomer, the IL-15 cytokine (I) is linked to the Fc domain, and the masking polypeptide (MP) is linked to the IL-15 cytokine (I) through the cleavable moiety (CM).

In some embodiments, the IL-15 prodrug provided herein comprises the constructs showing in Table 2.

In some embodiments, the IL-15 drug provided herein comprises two monomers, wherein the first monomer comprises the amino acid sequence of SEQ ID NO: 31 or a variant thereof having at least about 80% (such as at least about any one of 80%, 85%, 90%, 95%, 96%, 97%, 98%, or 99%) sequence identity to the amino acid sequence of SEQ ID NO: 31, and the second monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein the first monomer comprises the amino acid sequence of SEQ ID NO: 33 or a variant thereof having at least about 80% (such as at least about any one of 80%, 85%, 90%, 95%, 96%, 97%, 98%, or 99%) sequence identity to the amino acid sequence of SEQ ID NO: 33, and the second monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein the first monomer comprises the amino acid sequence of SEQ ID NO: 34 or a variant thereof having at least about 80% (such as at least about any one of 80%, 85%, 90%, 95%, 96%, 97%, 98%, or 99%) sequence identity to the amino acid sequence of SEQ ID NO: 34, and the second monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein the first monomer comprises the amino acid sequence of SEQ ID NO: 36 or a variant thereof having at least about 80% (such as at least about any one of 80%, 85%, 90%, 95%, 96%, 97%, 98%, or 99%) sequence identity to the amino acid sequence of SEQ ID NO: 36, and the second monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein the first monomer comprises the amino acid sequence of SEQ ID NO: 37 or a variant thereof having at least about 80% (such as at least about any one of 80%, 85%, 90%, 95%, 96%, 97%, 98%, or 99%) sequence identity to the amino acid sequence of SEQ ID NO: 37, and the second monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein the first monomer comprises the amino acid sequence of SEQ ID NO: 38 or a variant thereof having at least about 80% (such as at least about any one of 80%, 85%, 90%, 95%, 96%, 97%, 98%, or 99%) sequence identity to the amino acid sequence of SEQ ID NO: 38, and the second monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein the first monomer comprises the amino acid sequence of SEQ ID NO: 39 or a variant thereof having at least about 80% (such as at least about any one of 80%, 85%, 90%, 95%, 96%, 97%, 98%, or 99%) sequence identity to the amino acid sequence of SEQ ID NO: 39, and the second monomer comprises the amino acid sequence of SEQ ID NO: 32 or a variant thereof having at least about 80% (such as at least about any one of 80%, 85%, 90%, 95%, 96%, 97%, 98%, or 99%) sequence identity to the amino acid sequence SEQ ID NO: 32.

In some embodiments, the IL-15 prodrug provided herein comprises two monomers, wherein the first monomer comprises the amino acid sequence of SEQ ID NO: 45 or a variant thereof having at least about 80% (such as at least about any one of 80%, 85%, 90%, 95%, 96%, 97%, 98%, or 99%) sequence identity to the amino acid sequence of SEQ ID NO: 45, and the second monomer comprises the amino acid sequence of SEQ ID NO: 46 or a variant thereof having at least about 80% (such as at least about any one of 80%, 85%, 90%, 95%, 96%, 97%, 98%, or 99%) sequence identity to the amino acid sequence SEQ ID NO: 46.

In some embodiments, the IL-15 prodrug provided herein comprises two monomers, wherein the first monomer comprises the amino acid sequence of SEQ ID NO: 47 or a variant thereof having at least about 80% (such as at least about any one of 80%, 85%, 90%, 95%, 96%, 97%, 98%, or 99%) sequence identity to the amino acid sequence of SEQ ID NO: 47, and the second monomer comprises the amino acid sequence of SEQ ID NO: 40 or a variant thereof having at least about 80% (such as at least about any one of 80%, 85%, 90%, 95%, 96%, 97%, 98%, or 99%) sequence identity to the amino acid sequence SEQ ID NO: 40.

In some embodiments, the IL-15 prodrug provided herein comprises two monomers, wherein the first monomer comprises the amino acid sequence of SEQ ID NO: 33 or a variant thereof having at least about 80% (such as at least about any one of 80%, 85%, 90%, 95%, 96%, 97%, 98%, or 99%) sequence identity to the amino acid sequence of SEQ ID NO: 33, and the second monomer comprises the amino acid sequence of SEQ ID NO: 40 or a variant thereof having at least about 80% (such as at least about any one of 80%, 85%, 90%, 95%, 96%, 97%, 98%, or 99%) sequence identity to the amino acid sequence SEQ ID NO: 40.

In some embodiments, the IL-15 prodrug provided herein comprises two monomers, wherein the first monomer comprises the amino acid sequence of SEQ ID NO: 62 or a variant thereof having at least about 80% (such as at least about any one of 80%, 85%, 90%, 95%, 96%, 97%, 98%, or 99%) sequence identity to the amino acid sequence of SEQ ID NO: 62, and the second monomer comprises the amino acid sequence of SEQ ID NO: 63 or a variant thereof having at least about 80% (such as at least about any one of 80%, 85%, 90%, 95%, 96%, 97%, 98%, or 99%) sequence identity to the amino acid sequence SEQ ID NO: 63.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 63 or a variant thereof having at least about 80% (such as at least about any one of 80%, 85%, 90%, 95%, 96%, 97%, 98%, or 99%) sequence identity to the amino acid sequence SEQ ID NO: 63.

In some embodiments, the IL-15 prodrug provided herein comprises two monomers, wherein the first monomer comprises the amino acid sequence of SEQ ID NO: 64 or a variant thereof having at least about 80% (such as at least about any one of 80%, 85%, 90%, 95%, 96%, 97%, 98%, or 99%) sequence identity to the amino acid sequence of SEQ ID NO: 64, and the second monomer comprises the amino acid sequence of SEQ ID NO: 65 or a variant thereof having at least about 80% (such as at least about any one of 80%, 85%, 90%, 95%, 96%, 97%, 98%, or 99%) sequence identity to the amino acid sequence SEQ ID NO: 65.

In some embodiments, the IL-15 prodrug provided herein comprises two monomers, wherein the first monomer comprises the amino acid sequence of SEQ ID NO: 62 or a variant thereof having at least about 80% (such as at least about any one of 80%, 85%, 90%, 95%, 96%, 97%, 98%, or 99%) sequence identity to the amino acid sequence of SEQ ID NO: 62, and the second monomer comprises the amino acid sequence of SEQ ID NO: 65 or a variant thereof having at least about 80% (such as at least about any one of 80%, 85%, 90%, 95%, 96%, 97%, 98%, or 99%) sequence identity to the amino acid sequence SEQ ID NO: 65.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 65 or a variant thereof having at least about 80% (such as at least about any one of 80%, 85%, 90%, 95%, 96%, 97%, 98%, or 99%) sequence identity to the amino acid sequence SEQ ID NO: 65.

In some embodiments, the IL-15 prodrug provided herein comprises two monomers, wherein the first monomer comprises the amino acid sequence of SEQ ID NO: 66 or a variant thereof having at least about 80% (such as at least about any one of 80%, 85%, 90%, 95%, 96%, 97%, 98%, or 99%) sequence identity to the amino acid sequence of SEQ ID NO: 66, and the second monomer comprises the amino acid sequence of SEQ ID NO: 65 or a variant thereof having at least about 80% (such as at least about any one of 80%, 85%, 90%, 95%, 96%, 97%, 98%, or 99%) sequence identity to the amino acid sequence SEQ ID NO: 65.

In some embodiments, the IL-15 prodrug provided herein comprises two monomers, wherein the first monomer comprises the amino acid sequence of SEQ ID NO: 43 or a variant thereof having at least about 80% (such as at least about any one of 80%, 85%, 90%, 95%, 96%, 97%, 98%, or 99%) sequence identity to the amino acid sequence of SEQ ID NO: 43, and the second monomer comprises the amino acid sequence of SEQ ID NO: 46 or a variant thereof having at least about 80% (such as at least about any one of 80%, 85%, 90%, 95%, 96%, 97%, 98%, or 99%) sequence identity to the amino acid sequence SEQ ID NO: 46.

In some embodiments, the IL-15 prodrug provided herein comprises two monomers, wherein the first monomer comprises the amino acid sequence of SEQ ID NO: 39 or a variant thereof having at least about 80% (such as at least about any one of 80%, 85%, 90%, 95%, 96%, 97%, 98%, or 99%) sequence identity to the amino acid sequence of SEQ ID NO: 39, and the second monomer comprises the amino acid sequence of SEQ ID NO: 40 or a variant thereof having at least about 80% (such as at least about any one of 80%, 85%, 90%, 95%, 96%, 97%, 98%, or 99%) sequence identity to the amino acid sequence SEQ ID NO: 40.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 87 or a variant thereof having at least about 80% (such as at least about any one of 80%, 85%, 90%, 95%, 96%, 97%, 98%, or 99%) sequence identity to the amino acid sequence SEQ ID NO: 87.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 88 or a variant thereof having at least about 80% (such as at least about any one of 80%, 85%, 90%, 95%, 96%, 97%, 98%, or 99%) sequence identity to the amino acid sequence SEQ ID NO: 88.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 89 or a variant thereof having at least about 80% (such as at least about any one of 80%, 85%, 90%, 95%, 96%, 97%, 98%, or 99%) sequence identity to the amino acid sequence SEQ ID NO: 89.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 90 or a variant thereof having at least about 80% (such as at least about any one of 80%, 85%, 90%, 95%, 96%, 97%, 98%, or 99%) sequence identity to the amino acid sequence SEQ ID NO: 90.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 91 or a variant thereof having at least about 80% (such as at least about any one of 80%, 85%, 90%, 95%, 96%, 97%, 98%, or 99%) sequence identity to the amino acid sequence SEQ ID NO: 91.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 92 or a variant thereof having at least about 80% (such as at least about any one of 80%, 85%, 90%, 95%, 96%, 97%, 98%, or 99%) sequence identity to the amino acid sequence SEQ ID NO: 92.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 93 or a variant thereof having at least about 80% (such as at least about any one of 80%, 85%, 90%, 95%, 96%, 97%, 98%, or 99%) sequence identity to the amino acid sequence SEQ ID NO: 93.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 94 or a variant thereof having at least about 80% (such as at least about any one of 80%, 85%, 90%, 95%, 96%, 97%, 98%, or 99%) sequence identity to the amino acid sequence SEQ ID NO: 94.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 95 or a variant thereof having at least about 80% (such as at least about any one of 80%, 85%, 90%, 95%, 96%, 97%, 98%, or 99%) sequence identity to the amino acid sequence SEQ ID NO: 95.

In some embodiments, the IL-15 prodrug provided herein comprises two monomers, wherein the first monomer comprises the amino acid sequence of SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and the second monomer comprises the amino acid sequence of SEQ ID NO: 96 or a variant thereof having at least about 80% (such as at least about any one of 80%, 85%, 90%, 95%, 96%, 97%, 98%, or 99%) sequence identity to the amino acid sequence SEQ ID NO: 96.

In another aspect, the application also provided an IL-15 drug without masking polypeptide (MP) and cleavable moiety (CM) which comprises two monomers, wherein in one monomer, the IL-15Rα or a functional fragment thereof (S) is linked to the first Fc domain, and in the other monomer, the IL-15 cytokine (I) is linked to the second Fc domain.

In some embodiments, the IL-15 drug provided herein comprises two monomers, wherein the first monomer comprises the amino acid sequence of SEQ ID NO: 43 or a variant thereof having at least about 80% (such as at least about any one of 80%, 85%, 90%, 95%, 96%, 97%, 98%, or 99%) sequence identity to the amino acid sequence of SEQ ID NO: 43, and the second monomer comprises the amino acid sequence of SEQ ID NO: 77 or a variant thereof having at least about 80% (such as at least about any one of 80%, 85%, 90%, 95%, 96%, 97%, 98%, or 99%) sequence identity to the amino acid sequence SEQ ID NO: 77.

In some embodiments, the IL-15 drug provided herein comprises two monomers, wherein the first monomer comprises the amino acid sequence of SEQ ID NO: 43 or a variant thereof having at least about 80% (such as at least about any one of 80%, 85%, 90%, 95%, 96%, 97%, 98%, or 99%) sequence identity to the amino acid sequence of SEQ ID NO: 43, and the second monomer comprises the amino acid sequence of SEQ ID NO: 81 or a variant thereof having at least about 80% (such as at least about any one of 80%, 85%, 90%, 95%, 96%, 97%, 98%, or 99%) sequence identity to the amino acid sequence SEQ ID NO: 81.

In some embodiments, the IL-15 drug provided herein comprises two monomers, wherein the first monomer comprises the amino acid sequence of SEQ ID NO: 43 or a variant thereof having at least about 80% (such as at least about any one of 80%, 85%, 90%, 95%, 96%, 97%, 98%, or 99%) sequence identity to the amino acid sequence of SEQ ID NO: 43, and the second monomer comprises the amino acid sequence of SEQ ID NO: 83 or a variant thereof having at least about 80% (such as at least about any one of 80%, 85%, 90%, 95%, 96%, 97%, 98%, or 99%) sequence identity to the amino acid sequence SEQ ID NO: 83.

In some embodiments, the IL-15 drug provided herein comprises two monomers, wherein the first monomer comprises the amino acid sequence of SEQ ID NO: 43 or a variant thereof having at least about 80% (such as at least about any one of 80%, 85%, 90%, 95%, 96%, 97%, 98%, or 99%) sequence identity to the amino acid sequence of SEQ ID NO: 43, and the second monomer comprises the amino acid sequence of SEQ ID NO: 84 or a variant thereof having at least about 80% (such as at least about any one of 80%, 85%, 90%, 95%, 96%, 97%, 98%, or 99%) sequence identity to the amino acid sequence SEQ ID NO: 84.

### Binding Affinity

Binding affinity of a molecule (e.g., IL-15 or functional fragment thereof) and its binding partner (e.g., IL-2/IL-15Rβγ) can be determined experimentally by any suitable ligand binding assays or antibody/antigen binding assays known in the art, e.g., Western blots, sandwich enzyme-linked immunosorbent assay (ELISA), Meso Scale Discovery (MSD) electrochemiluminescence, bead based multiplex immunoassays (MIA), RIA, Surface Plasma Resonance (SPR), ECL, IRMA, EIA, Biacore assay, Octet analysis, peptide scans, etc. For example, easy analysis is possible by using an IL-15 or functional fragment thereof, or its receptor (e.g., IL-2/IL-15Rβγ) or subunits thereof marked with a variety of marker agents, as well as by using BiacoreX (Amersham Biosciences), which is an over-the-counter, measuring kit, or similar kit, according to the user's manual and experiment operation method attached with the kit.

In some embodiments, protein microarray is used for analyzing the interaction, function, and activity of the IL-15 or functional fragment thereof, described herein to its receptor, on a large scale. The protein chip has a support surface-bound with a range of capture proteins (e.g., IL-15 receptor or subunits thereof). Fluorescently labeled probe molecules (e.g., IL-15 or functional fragment thereof described herein) are then added to the array and upon interaction with the bound capture protein, a fluorescent signal is released and read by a laser scanner.

Binding affinity can also be measured using SPR (Biacore T-200). For example, anti-human IgG antibody is coupled to the surface of a CM-5 sensor chip using EDC/NHS chemistry. Then human IL-2/IL-15Rβγ-Fc fusion protein is used as the captured ligand over this surface. Serial dilutions of IL-15 prodrug or drug described herein are allowed to bind to the captured ligands, and the binding and dissociation of IL-15 prodrug or drug to IL-2/IL-15Rβγ can be monitored in real time. Equilibrium dissociation constant (Kd) and dissociation rate constant can be determined by performing kinetic analysis using BIA evaluation software.

### Pharmacokinetics (PK)

Pharmacokinetics (PK) refers to the absorption, distribution, metabolism, and excretion of a drug (e.g., IL-15 cytokine or IL-15 prodrug described herein) once it has been administered to a subject. Pharmacokinetic parameters that may be useful in determining clinical utility include but are not limited to serum/plasma concentration, serum/plasma concentration over time, maximum serum/plasma concentration (Cₘₐₓ), time to reach maximum concentration (Tₘₐₓ), half-life (t_{1/2}), area under concentration time curve within the dosing interval (AUC_{τ}), etc.

Techniques for obtaining a PK curve of a drug, such as IL-15 cytokine or IL-15 prodrug described herein, are known in the art. See, e.g., Heller et al., Annu Rev Anal Chem, 11, 2018; and Ghandforoush-Sattari et al., J Amino Acids, Article ID 346237, Volume 2010. In some embodiments, the PK curves of the IL-15 cytokine or IL-15 prodrug described herein in the individual is measured in a blood, plasma, or serum sample from the individual. In some embodiments, the PK curves of the IL-15 cytokine or IL-15 prodrug described herein in the individual is measured using a mass spectrometry technique, such as LC-MS/MS, or ELISA. PK analysis on PK curves can be conducted by any methods known in the art, such as non-compartmental analysis, e.g., using PKSolver V2 software (Zhang Y. et al., “PKSolver: An add-in program for pharmacokinetic and pharmacodynamic data analysis in Microsoft Excel,” Comput Methods Programs Biomed. 2010; 99(3):306-1).

“C” denotes the concentration of drug or prodrug (e.g., IL-15 cytokine or IL-15 prodrug) in blood plasma, serum, or in any appropriate body fluid or tissue of a subject, and is generally expressed as mass per unit volume, for example nanograms per milliliter. For convenience, the concentration of drug in serum or plasma is referred to herein as “serum concentration” or “plasma concentration.” The serum/plasma concentration at any time following drug administration (e.g., IL-15 cytokine or IL-15 prodrug, such as i.v., i.p., or s.c. administration) is referenced as Cₜᵢₘₑ or Cₜ. The maximum serum/plasma drug concentration during the dosing period is referenced as Cₘₐₓ, while Cₘᵢₙ refers to the minimum serum/plasma drug concentration at the end of a dosing interval; and Cₐᵥₑ refers to an average concentration during the dosing interval.

The term “bioavailability” refers to an extent to which and sometimes rate at which the drug or prodrug (e.g., IL-15 cytokine or IL-15 prodrug) enters systemic circulation, thereby gaining access to the site of action.

“AUC” is the area under the serum/plasma concentration-time curve and is considered to be the most reliable measure of bioavailability, such as area under concentration time curve within the dosing interval (AUC_{τ}), “overall exposure” or “total drug exposure across time” (AUC₀₋ₗₐₛₜ or AUC_{0-inf}), area under concentration time curve at time t post-administration (AUC₀₋ₜ), etc.

Serum/plasma concentration peak time (Tₘₐₓ) is the time when peak serum/plasma concentration (Cₘₐₓ) is reached after administration of a drug or prodrug (e.g., IL-15 cytokine or IL-15 prodrug).

Half-life (t_{1/2}) is the amount of time required for the drug or prodrug concentration (e.g., IL-15 cytokine or IL-15 prodrug) measured in plasma or serum (or other biological matrices) to be reduced to exactly half of its concentration or amount at certain time point. For example, after iv dosing, the drug concentrations in plasma or serum decline due to both distribution and elimination. In a plasma or serum profile of drug concentration over time post-iv doing, the first phase or rapid decline is considered to be primarily due to distribution, while the later phase of decline is usually slower and considered to be primarily due to elimination, although both processes occur in both phases. Distribution is assumed to be complete after sufficient time. In general, the elimination half-life is determined from the terminal or elimination (dominant) phase of the plasma/serum concentration versus time curve. See, e.g., Michael Schrag and Kelly Regal, “Chapter 3—Pharmacokinetics and Toxicokinetics” of “A Comprehensive Guide to Toxicology in Preclinical Drug Development”, 2013.

### Stability

In some embodiments, the masking polypeptide (MP) described herein and the prodrug (e.g., IL-15 prodrug, anti-TNFR2 antibody prodrug) described herein have excellent stability, such as physical stability, chemical stability, and/or biological stability. In some embodiments, the IL-15 prodrug and anti-TNFR2 antibody prodrug described herein have superior stability under accelerated stress (e.g., high temperature), such as less or no fragmentation, aggregate formation, and/or aggregate increment.

Stability of protein, in particular the susceptibility to aggregation, is primarily determined by the conformational and the colloidal stability of the protein molecules. It is generally believed that the first step in non-native protein aggregation, which is the most prevalent form of aggregation, is a slight perturbation of the molecular structure, e.g., a partial unfolding of the protein, i.e., a conformational change. This is determined by the conformational stability of the protein. In the second step, the partially unfolded molecules then come into close proximity, being driven by diffusion and random Brownian motion, to form aggregates. This second step is primarily governed by the colloidal stability of the molecules (see Chi et al., Roles of conformational stability and colloidal stability in the aggregation of recombinant human granulocyte colony stimulating factor. Protein Science, 2003 May; 12(5): 903-913). As used herein, the term “stability” generally is related to maintaining the integrity or to minimizing the degradation, denaturation, aggregation or unfolding of a biologically active agent such as a protein. As used herein, “improved stability” generally means that, under conditions known to result in degradation, denaturation, aggregation or unfolding, the protein (e.g., IL-15 prodrug described herein) of interest maintains greater stability compared to a control protein (e.g., other IL-15 prodrug).

Differential scanning calorimetry (DSC) and differential scanning fluorimetry (DSF) are well known techniques in the art that are used to predict the stability of a protein formulation. Specifically, these techniques can be used to determine the unfolding temperature (Tm) of a protein in given formulation. It is standard in the art to correlate high Tm measurements for a protein in given formulation with more robust and stable protein formulations for long-term, shelf-stable storage.

A “stable” masking polypeptide (MP) or prodrug (or formulation), e.g., IL-15 prodrug or an anti-TNFR2 antibody prodrug described herein, essentially retains its physical stability and/or chemical stability and/or biological activity during the manufacturing process and/or upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. (1993) Adv. Drug Delivery Rev. 10: 29-90. For example, in one embodiment, the stability of the protein is determined according to the percentage of monomer protein in the solution, with a low percentage of degraded (e.g., fragmented) and/or aggregated protein. Preferably, the protein (or formulation) is stable at room temperature (about 30° C.) or at 40° C. for at least 1 month and/or stable at about 2-8° C. for at least 6 months, or for at least 1 year or for at least 2 years. Furthermore, the protein (or formulation) is preferably stable following freezing (to, e.g., −70° C.) and thawing, hereinafter referred to as a “freeze/thaw cycle.”

A prodrug, e.g., IL-15 prodrug or an anti-TNFR2 antibody prodrug described herein, “retains its physical stability” in a formulation if it shows substantially no signs of instability, e.g., aggregation, precipitation and/or denaturation, upon visual examination of color and/or clarity or as measured by UV light scattering or by size exclusion chromatography. Aggregation is a process whereby individual protein molecules or complexes associate covalently or non-covalently to form aggregates. Aggregation can proceed to the extent that a visible precipitate is formed.

A prodrug, e.g., IL-15 prodrug or an anti-TNFR2 antibody prodrug described herein, “retains its chemical stability” in a formulation, if the chemical stability at a given time is such that the protein is considered to still retain its biological activity (e.g., as mentioned in “Bioactivity” subsection above). Chemical stability can be assessed by, e.g., detecting and quantifying chemically altered forms of the protein. Chemical alteration may involve size modification (e.g., clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g., occurring as a result of deamidation or oxidation) which can be evaluated by ion-exchange chromatography, for example.

A prodrug, e.g., IL-15 prodrug or an anti-TNFR2 antibody prodrug described herein, “retains its biological activity” in a formulation, if the protein, in a pharmaceutical formulation is biologically active for its intended purpose. For example, biological activity is retained if the biological activity of the protein, in the formulation is within about 30%, about 20%, or about 10% (within the errors of the assay) of the biological activity exhibited at the time the formulation was prepared.

One of skilled in the art will appreciate that stability of a prodrug (e.g., IL-15 prodrug or an anti-TNFR2 antibody prodrug described herein) is dependent on other features in addition to the composition of the formulation. For example, stability can be affected by temperature, pressure, humidity, pH, and external forms of radiation. Stability of a protein (e.g., IL-15 prodrug or an anti-TNFR2 antibody prodrug) in a protein formulation can be determined by various means. In some embodiments, the protein stability is determined by size exclusion chromatography (SEC). SEC separates analytes (e.g., macromolecules such as proteins) on the basis of a combination of their hydrodynamic size, diffusion coefficient, and surface properties. Thus, for example, SEC can separate IL-15 prodrug or an anti-TNFR2 antibody prodrug described herein in their natural three-dimensional conformation from proteins in various states of denaturation, and/or proteins that have been degraded. In SEC, the stationary phase is generally composed of inert particles packed into a dense three-dimensional matrix within a glass or steel column. The mobile phase can be pure water, an aqueous buffer, an organic solvent, mixtures of these, or other solvents. The stationary-phase particles have small pores and/or channels which will only allow species below a certain size to enter. Large particles are therefore excluded from these pores and channels, but the smaller particles are removed from the flowing mobile phase. The time particles spend immobilized in the stationary-phase pores depends, in part, on how far into the pores they can penetrate. Their removal from the mobile phase flow causes them to take longer to elute from the column and results in a separation between the particles based on differences in their size.

In some embodiments, SEC is combined with an identification technique to identify or characterize proteins (e.g., IL-15 prodrug described herein or an anti-TNFR2 antibody prodrug), or fragments thereof. Protein identification and characterization can be accomplished by various techniques, including but not limited chromatographic techniques, e.g., high-performance liquid chromatography (HPLC), Capillary Electrophoresis-Sodium Dodecyl Sulfate (CE-SDS), immunoassays, electrophoresis, ultra-violet/visible/infrared spectroscopy, raman spectroscopy, surface enhanced raman spectroscopy, mass spectroscopy, gas chromatography, static light scattering (SLS), Fourier Transform Infrared Spectroscopy (FTIR), circular dichroism (CD), urea-induced protein unfolding techniques, intrinsic tryptophan fluorescence, differential scanning calorimetry, and/or ANS protein binding.

In some embodiments, sample formulations (e.g., comprising the IL-15 prodrug or an anti-TNFR2 antibody prodrug described herein) and reference formulations are optionally assayed prior to a treatment phase to determine the content of monomer, aggregated and/or fragmented protein (and/or fragmentation increase %, aggregation increase %, etc.). Subsequently, each of the protein formulations undergoes a treatment phase. For example, each protein formulation may be stored for an extended period (e.g., 3 months, 6 months, 12 months, or longer) at a specific temperature (e.g., 40° C., 25° C., or 5° C.). In some embodiments, the protein formulations undergo a physical stress test such as stir-stress assay. In some embodiments, the protein formulations undergo accelerated stability test, such as treated under accelerated stress, including high temperature (e.g., 40° C.), high humidity, and/or low pH, etc. In some embodiments, the protein formulations undergo cycles of freezing and thawing. In some embodiments, samples of the same protein formulation receive differential treatment, e.g., storage for a period of time in different temperatures. Following the treatment phase, the protein formulations are assayed to determine the content of protein monomer, aggregates and/or fragments (and/or fragmentation increase %, aggregation increase %, etc.).

“Substantial protein aggregation” refers to a level of protein aggregation in a protein formulation that is substantially greater than the level of protein aggregation in a reference protein formulation. The reference protein formulation may be the same protein formulation before a period of storage or before a treatment (e.g., before subjected to a destabilizing condition, such as elevated temperature, humidity, pH, and/or to long term storage.).

“Substantially free of protein aggregation” refers to proteins (or formulations) of the application that do not have a significantly greater level or percentage of aggregated protein than a reference formulation. In some embodiments, the stability is measured by SEC. In some embodiments, the stability is measured by CE-SDS.

In some embodiments, stability refers to reduced fragmentation of the IL-15 prodrug or an anti-TNFR2 antibody prodrug described herein. The term “low to undetectable levels of fragmentation” as used herein refers to samples containing equal to or more than 80%, 85%, 90%, 95%, 98% or 99% of the total protein, for example, in a single peak as determined by HPSEC, or in multiple peaks (e.g., as many peaks as there are subunits) by reduced Capillary Gel Electrophoresis (rCGE), representing the non-degraded protein or a non-degraded fragment thereof, and containing no other single peaks having more than 5%, more than 4%, more than 3%, more than 2%, more than 1%, or more than 0.5% of the total protein in each. The term “reduced Capillary Gel Electrophoresis” as used herein refers to capillary gel electrophoresis under reducing conditions sufficient to reduce disulfide bonds in an Fc-containing protein, such as the IL-15 prodrug or anti-TNFR2 antibody prodrug described herein.

### Vectors

The present application also provides isolated nucleic acids encoding any of the masking polypeptides (MP), any of the cleavable moieties (CM), any of the non-cleavable linker (L) or any of the prodrugs (e.g., anti-TNFR2 antibody prodrug or IL-15 prodrug) described herein, vectors comprising the nucleic acids described herein. Also provided are isolated host cells (e.g., CHO cells, HEK 293 cells, Hela cells, or COS cells) comprising nucleic acids or vectors described herein. Suitable nucleic acid constructs include, but are not limited to, constructs that are capable of expression in prokaryotic or eukaryotic cells. Expression constructs are generally selected so as to be compatible with the host cell in which they are to be used. In some embodiments, the vector encodes a masking polypeptide (e.g., MP80, MP96new, MP100, MP163 or MP240). In some embodiments, the vector encodes a cleavable moiety (e.g., CM1, CM2 or CM4). In some embodiments, the vector encodes a non-cleavable linker (e.g., lk, lk1, lk2, lk3 or lk5). In some embodiments, the vector encodes a protein or prodrugs (e.g., masking polypeptides or IL-15 prodrug).

In some embodiments, the vector comprising a nucleic acid encoding the prodrug or any components of the prodrugs described herein is suitable for replication and integration in eukaryotic cells, such as mammalian cells (e.g., CHO cells, HEK 293 cells, Hela cells, COS cells). In some embodiments, the vector is a viral vector. In some embodiments, the vector is a non-viral vector, such as pTT5.

A number of viral based systems have been developed for gene transfer into mammalian cells. Examples of viral vectors include, but are not limited to, adenoviral vectors, adeno-associated virus vectors, lentiviral vectors, retroviral vectors, herpes simplex viral vectors, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals. Retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the construct protein coding sequence(s) can be packaged with protocols known in the art. The resulting lentiviral vectors can be used to transduce a mammalian cell using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a pTT5 vector. In some embodiments, the vector is a transposon, such as a Sleeping Beauty (SB) transposon system, or a PiggyBac transposon system. In some embodiments, the vector is a polymer-based non-viral vector, including for example, poly (lactic-co-glycolic acid) (PLGA) and poly lactic acid (PLA), poly (ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as Poly-L-lysine. Any of the known non-viral vectors suitable for genome editing can be used for introducing the IL-15 prodrug-encoding nucleic acid(s) to the host cells. See, for example, Yin H. et al., Nature Rev. Genetics (2014) 15:521-555; Aronovich E L et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5(1): 120-125, which are incorporated herein by reference. In some embodiments, any one or more of the nucleic acids or vectors encoding the prodrugs described herein is introduced to the host cells (e.g., CHO, HEK 293, Hela, or COS) by a physical method, including, but not limited to electroporation, sonoporation, photoporation, magnetofection, hydroporation.

In some embodiments, the vector contains a selectable marker gene or a reporter gene to select cells expressing the prodrugs described herein from the population of host cells transfected through vectors (e.g., lentiviral vectors, pTT5 vectors). Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in prokaryotic cells or eukaryotic cells (e.g., mammalian cells), and any of the promoters known in the art may be used in the present application. Promoters may be roughly categorized as constitutive promoters or regulated promoters, such as inducible promoters.

In some embodiments, the nucleic acid encoding the prodrugs described herein is operably linked to a constitutive promoter. Constitutive promoters allow heterologous genes (also referred to as transgenes) to be expressed constitutively in the host cells. Exemplary promoters contemplated herein include, but are not limited to, cytomegalovirus immediate-early promoter (CMV), human elongation factors-1alpha (hEF1α), ubiquitin C promoter (UbiC), phosphoglycerokinase promoter (PGK), simian virus 40 early promoter (SV40), chicken β-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The efficiencies of such constitutive promoters on driving transgene expression have been widely compared in a huge number of studies. In some embodiments, the nucleic acid encoding the prodrugs described herein is operably linked to CMV promoter.

In some embodiments, the nucleic acid encoding the prodrugs described herein is operably linked to an inducible promoter. Inducible promoters belong to the category of regulated promoters. The inducible promoter can be induced by one or more conditions, such as a physical condition, microenvironment of the host cells, or the physiological state of the host cells, an inducer (i.e., an inducing agent), or a combination thereof. In some embodiments, the inducing condition does not induce the expression of endogenous genes in the host cell. In some embodiments, the inducing condition is selected from the group consisting of inducer, irradiation (such as ionizing radiation, light), temperature (such as heat), redox state, and the activation state of the host cell. In some embodiments, the inducible promoter can be an NFAT promoter, a TETON® promoter, or an NFκB promoter.

### Methods of Preparation

Also provided are methods of preparing any of the masking polypeptides (MP), the cleavable moieties (CM), the non-cleavable linkers (L) or prodrugs described herein. Thus in some embodiments, there is provided a method of producing the masking polypeptides (MP), the cleavable moieties (CM), the non-cleavable linkers (L) or the prodrugs, comprising: (a) culturing a host cell (e.g., CHO cell, HEK 293 cell, Hela cell, or COS cell) comprising any of the nucleic acids or vectors encoding the masking polypeptides (MP), the cleavable moieties (CM), the non-cleavable linkers (L) or the prodrugs described herein under a condition effective to express the encoded prodrug; and (b) obtaining the expressed the masking polypeptides (MP), the cleavable moieties (CM), the non-cleavable linkers (L) or prodrugs from said host cell. In some embodiments, the method of step (a) further comprises producing a host cell comprising the nucleic acid or vector encoding the masking polypeptides (MP), the cleavable moieties (CM), the non-cleavable linkers (L) or the prodrug described herein. The masking polypeptides (MP), the cleavable moieties (CM), the non-cleavable linkers (L) or the prodrugs described herein may be prepared using any methods known in the art or as described herein.

In some embodiments, the masking polypeptides (MP), the cleavable moieties (CM), the non-cleavable linkers (L) or the prodrugs described herein are expressed with eukaryotic cells, such as mammalian cells. In some embodiments, the masking polypeptides (MP), the cleavable moieties (CM), the non-cleavable linkers (L) or the prodrugs described herein are expressed with prokaryotic cells.

### 1. Recombinant Production in Prokaryotic Cells

### a) Vector Construction

Polynucleic acid sequences encoding the protein constructs of the present application can be obtained using standard recombinant techniques. Polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present application. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector, which can be used to transform susceptible host cells such as E. coli LE392.

A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the polypeptide by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the—galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target light and heavy chains (Siebenlist et al. (1980) Cell 20: 269) using linkers or adaptors to supply any required restriction sites.

In some embodiments, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this application should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

In some embodiments, the production of the protein construct according to the present application can occur in the cytoplasm of the host cell, and therefore does not require the presence of secretion signal sequences within each cistron. In some embodiments, polypeptide components are expressed, folded, and assembled to form the protein construct within the cytoplasm. Certain host strains (e.g., the E. coli trxB⁻ strains) provide cytoplasm conditions that are favorable for disulfide bond formation, thereby permitting proper folding and assembly of expressed protein subunits. See Proba and Pluckthun, Gene, 159:203 (1995).

### b) Prokaryotic Host Cells

Prokaryotic host cells suitable for expressing the proteins of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In some embodiments, E. coli cells are used as hosts for the application. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kan^{R}(U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon.

Typically, the host cell should secrete minimal amounts of proteolytic enzymes, and additional protease inhibitors may desirably be incorporated in the cell culture.

### c) Protein Production

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the protein constructs of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol, and dithiothreitol. The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

The expressed protein constructs of the present application are secreted into and recovered from the periplasm of the host cells. Protein recovery typically involves disrupting the microorganism, generally by such means as osmotic shock, sonication, or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantities by a fermentation process. Various large-scale fed-batch fermentation procedures are available for the production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small-scale fermentation refers generally to fermentation in a fermentor that is no more than approximately 100 liters in volumetric capacity and can range from about 1 liter to about 100 liters.

During the fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the protein constructs of the present application, various fermentation conditions can be modified. For example, to improve the proper assembly and folding of the secreted polypeptides, additional vectors overexpressing chaperone proteins, such as Dsb proteins (DsbA, DsbB, DsbC, DsbD, or DsbG) or FkpA (a peptidylprolyl cis-, trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present application. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

E. coli strains deficient for proteolytic enzymes and transformed with plasmids overexpressing one or more chaperone proteins may be used as host cells in the expression system encoding the protein constructs of the present application.

### d) Protein Purification

The protein constructs produced herein are further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In some embodiments, Protein A immobilized on a solid phase is used for immunoaffinity purification of the protein constructs comprising an Fc region of the present application. Protein A is a 42 kDa surface protein from Staphylococcus aureus which binds with a high affinity to Fc-containing constructs, e.g., IL-15 prodrug or anti-TNFR2 antibody prodrug described herein. Lindmark et al (1983) J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants. The solid phase is then washed to remove contaminants non-specifically bound to the solid phase. Finally, the protein constructs of interest are recovered from the solid phase by elution.

### 2. Recombinant Production in Eukaryotic Cells

For eukaryotic expression, the vector components generally include, but are not limited to, one or more of the following, a signal sequence, an origin of replication, one or more marker genes, an enhancer element, a promoter, and a transcription termination sequence.

### a) Signal Sequence Component

A vector for use in a eukaryotic host may also be an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region is ligated in reading frame to DNA encoding the protein constructs of the present application.

### b) Origin of Replication

Generally, the origin of replication component is not needed for mammalian expression vectors (the SV40 origin may typically be used only because it contains the early promoter).

### c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the protein constructs of the present application, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

### d) Promoter Component

Expression and cloning vectors usually contain a promoter that is recognized by the host organism and is operably linked to the nucleic acid encoding the desired polypeptide sequences. Virtually all eukaryotic genes have an AT-rich region located approximately 25 to 30 based upstream from the site where transcription is initiated. Another sequence found 70 to 80 bases upstream from the start of the transcription of many genes is a CNCAAT region where N may be any nucleotide. At the 3′ end of most eukaryotic is an AATAAA sequence that may be the signal for addition of the poly A tail to the 3′ end of the coding sequence. All of these sequences may be inserted into eukaryotic expression vectors. Also see section “Vectors” above.

Polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human-interferon cDNA in mouse cells under the control of a thymidine kinase promoter from herpes simplex virus. Alternatively, the Rous Sarcoma Virus long terminal repeat can be used as the promoter.

### e) Enhancer Element Component

Transcription of a DNA encoding the protein constructs of the present application by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (100-270 bp), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

### f) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region. See WO94/11026 and the expression vector disclosed therein.

### g) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); COS fibroblast-like cell lines derived from monkey kidney tissue; human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for protein construct production and cultured in conventional nutrient media modified as appropriate for inducing promoters, selecting transformants, or amplifying the genes encoding the desired sequences.

### h) Culturing the Host Cells

The host cells used to produce the protein constructs of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression and will be apparent to the ordinarily skilled artisan.

### i) Protein Purification

When using recombinant techniques, the protein constructs of the present application can be produced intracellularly, in the periplasmic space, or directly secreted into the medium. If the protein construct is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the protein construct is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the Fc-containing protein construct. Protein A can be used to purify Fc-containing proteins based on human immunoglobulins containing 1, 2, or 4 heavy chains (Lindmark et al., J. Immunol. Meth. 62:1-13 (1983)). Protein G is recommended for all mouse isotypes and for human 3 (Guss et al., EMBO J. 5:15671575 (1986)). The matrix to which the affinity ligand is attached is most often agarose, but other matrices are available. Mechanically stable matrices such as controlled pore glass or poly(styrene-divinyl) benzene allow for faster flow rates and shorter processing times than can be achieved with agarose. Where the protein construct comprises a C_{H}3 domain, the Bakerbond ABXTMresin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the protein construct to be recovered.

Following any preliminary purification step(s), the mixture comprising the protein constructs of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Compositions

Further provided are pharmaceutical compositions comprising prodrugs (e.g., IL-15 prodrug or anti-TNFR2 antibody prodrug) described herein, and optionally a pharmaceutically acceptable carrier. Pharmaceutical compositions can be prepared by mixing a prodrug described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

A reconstituted formulation can be prepared by dissolving a lyophilized prodrug in a diluent such that the protein is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g., phosphate-buffered saline), sterile saline solution, Ringer's solution or dextrose solution, or aqueous solutions of salts and/or buffers.

In some embodiments, the pharmaceutical composition comprises a homogeneous population of prodrugs (e.g., IL-15 prodrug) described herein. A homogeneous population means the prodrugs are exactly the same to each other, e.g., same IL-15 prodrug configuration, same IL-15 cytokine, same IL-15Rα sushi domain, same masking polypeptides, same cleavable moiety, same non-cleavable linker if any, and same Fc domain. In some embodiments, at least about 70% (such as at least about any of 75%, 80%, 85%, 90%, 95%, 96%, 97%, 98%, or 99%) of the IL-15 prodrug in the pharmaceutical composition are homogeneous.

The pharmaceutical composition is preferably to be stable, in which the proteins contained within essentially retain their physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. For example, the extent of aggregation during storage can be used as an indicator of protein stability.

In some embodiments, the pharmaceutical composition has a shelf life of at least about 15 days, such as at least about any of 20 days, 1 month, 2 months, 3 months, 6 months, 1 year, 2 years, 3 years, or longer, for example, at about 2-25° C., such as about 2-8° C. As used herein, “shelf life” means that the storage period during which an active ingredient such as a therapeutic protein (e.g., the IL-15 prodrug described herein) in a pharmaceutical formulation has minimal degradation (e.g., not more than about 5% degradation, such as not more than about 4%, 3%, or 2% degradation) when the pharmaceutical formulation is stored under specified storage conditions, for example, 2-8° C. Exemplary techniques for assessing protein or formulation stability include size-exclusion chromatography (SEC)-HPLC to detect, e.g., aggregation, reverse phase (RP)-HPLC to detect, e.g. protein fragmentation, ion exchange-HPLC to detect, e.g., changes in the charge of the protein, mass spectrometry, fluorescence spectroscopy, circular dichroism (CD) spectroscopy, Fourier transform infrared spectroscopy (FT-IR), and Raman spectroscopy to detect protein conformational changes. All of these techniques can be used singly or in combination to assess the degradation of the protein in the pharmaceutical formulation and determine the shelf life of that formulation.

Acceptable carriers, excipients, or stabilizers are nontoxic to recipients at the dosages and concentrations employed and include buffers, antioxidants including ascorbic acid, methionine, Vitamin E, sodium metabisulfite; preservatives, isotonicifiers (e.g., sodium chloride), stabilizers, metal complexes (e.g. Zn-protein complexes); chelating agents such as EDTA and/or non-ionic surfactants.

Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, polyethylene glycol (PEG), and PLURONICS™.

Buffers are used to control the pH in a range which optimizes the therapeutic effectiveness, especially if stability is pH dependent. Suitable buffering agents for use in the present application include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth. The addition of a preservative may, for example, facilitate the production of a multi-use (multiple-dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1% to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the proteins as well as to protect the proteins against agitation-induced aggregation, which also permits the formulation to be exposed to shear surface stress without causing denaturation of the active proteins.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

For the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

Sustained-release preparations may be prepared. Suitable examples of sustained-release preparations include semi-permeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and Ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical compositions herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

### Methods of Treating Diseases

Further provided are methods of treating a subject with or at risk of developing a disease or disorder, such as proliferative disease, a tumorous disease, an inflammatory disease, an immunological disorder, an autoimmune disease, an infectious disease, a viral disease, an allergic reaction, a parasitic reaction, or graft-versus-host disease. The methods administering to a subject in need thereof an effective amount of an activatable prodrug as disclosed herein that is typically administered as a pharmaceutical composition, wherein the prodrug is activated upon cleavage by an enzyme. In some embodiments, the method further comprises selecting a subject with or at risk of developing such a disease or disorder. In some embodiments, the prodrug is activated in a tumor microenvironment. The prodrug is therapeutically active after it has cleaved from the masking polypeptides. Thus, in some embodiments, the active agent is the cleavage product. In some embodiments, the prodrugs can be used to treat a disease depending on the antigen bound by the antigen-binding domain.

In some embodiments, there is provided a method of treating a disease (e.g., a tumor, a viral infection, or a bacterial infection) in an individual (e.g., human), comprising administering to the individual an effective amount of any of the prodrugs (e.g., IL-15 prodrug or anti-TNFR2 antibody prodrug) described herein or pharmaceutical compositions thereof. In some embodiments, the prodrug (or a pharmaceutical composition thereof) is administered intravenously, intramuscularly, or subcutaneously. In some embodiments, the method of treatment further comprises administering an additional therapeutic agent in combination with (before, after, or concurrently with) the prodrug. The additional agent may be an antibody or antigen-binding fragment thereof, a small molecule drug, or other types of therapeutic drug.

In some embodiments, the IL-15 prodrug or the anti-TNFR2 antibody prodrug is used to treat a cancer or tumor in a subject comprises administering to the subject an effective amount of an IL-15 prodrug or anti-TNFR2 antibody prodrug. In some embodiments, as used herein, the term “tumor or cancer” refers to all types of cancer, neoplasm or malignant tumors found in mammals, including leukemias, lymphomas, melanomas, neuroendocrine tumors, carcinomas and sarcomas. Exemplary cancers that may be treated with a masked cytokine, pharmaceutical composition, or method provided herein, include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, Herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. nonsmall cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

In some embodiments, the IL-15 prodrug is used to treat a bacterial infection such as sepsis. In some embodiments, the bacteria causing the bacterial infection are drug-resistant bacteria. In some embodiments, the antigen-binding moiety binds to a bacterial antigen.

In some embodiments, the IL-15 prodrug is used to treat a viral infection. In some embodiments, the virus causing the viral infection is hepatitis C (HCV), hepatitis B (HBV), human immunodeficiency vims (HIV), or human papilloma virus (HPV). In some embodiments, the antigen-binding moiety binds to a viral antigen.

Administration of the prodrug described herein or pharmaceutical compositions thereof may be carried out in any convenient manner, including by injection or transfusion. The route of administration is in accordance with known and accepted methods, such as by single or multiple bolus or infusion over a long period of time in a suitable manner. The prodrug or pharmaceutical compositions thereof may be administered to a patient orally, subcutaneously, intravenously, intracerebrally, intranasally, transdermally, intraperitoneally, intramuscularly, intrapulmonarily, vaginally, rectally, intraocularly, topically, transarterially, intradermally, intranodally, intraputaminally, or intramedullary, intrathecally, intraventricularly, intracerebrally, intraspinally, intrathecially, intralesionally, or intraocularly. In some embodiments, the prodrug or pharmaceutical composition thereof is administered systemically. In some embodiments, the prodrug or pharmaceutical composition thereof is administered to an individual by infusion, such as intravenous infusion. Infusion techniques for immunotherapy are known in the art (see, e.g., Rosenberg et al., New Eng. J. of Med. 319: 1676 (1988)). In some embodiments, the prodrug or pharmaceutical composition thereof is administered to an individual by intradermal or subcutaneous (i.e. beneath the skin) injection. For subcutaneous injections, the prodrug or pharmaceutical composition thereof may be injected using a syringe. However, other devices for administration of the prodrug or pharmaceutical composition thereof are available such as injection devices; injector pens; auto-injector devices, needleless devices; and subcutaneous patch delivery systems. In some embodiments, the prodrug or pharmaceutical composition thereof is administered by intravenous injection. In some embodiments, the prodrug or pharmaceutical composition thereof is injected directly into the brain or spine. In some embodiments, the prodrug or pharmaceutical composition thereof is administered by sustained release or extended-release means.

Dosages and desired drug concentration of pharmaceutical compositions of the present application may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

When in vivo administration of the prodrug or pharmaceutical composition thereof are used, the dosage amounts may vary depending upon the route of administration and mammal type. It is within the scope of the present application that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

In some embodiments, the prodrug or pharmaceutical composition thereof is administered for a single time (e.g. bolus injection). In some embodiments, the prodrug or pharmaceutical composition thereof is administered for multiple times (such as any of 2, 3, 4, 5, 6, or more times). If multiple administrations, they may be performed by the same or different routes and may take place at the same site or at alternative sites. The prodrug or pharmaceutical composition thereof may be administered daily to once per year. The interval between administrations can be about any one of 24 hours to a year. Intervals can also be irregular (e.g. following tumor progression). In some embodiments, there is no break in the dosing schedule. The optimal dosage and treatment regime for a particular patient can readily be determined by one skilled in the art of medicine by monitoring the patient for signs of disease and adjusting the treatment accordingly.

In some embodiments, the prodrug or pharmaceutical composition thereof is administered in split doses, such as about any one of 2, 3, 4, 5, or more doses. In some embodiments, the split doses are administered over about a week, a month, 2 months, 3 months, or longer. In some embodiments, the dose is equally split. In some embodiments, the split doses are about 20%, about 30% and about 50% of the total dose. In some embodiments, the interval between consecutive split doses is about 1 day, 2 days, 3 days, 1 week, 2 weeks, 3 weeks, a month, 3 months, 6 months, or longer. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Articles of Manufacture and Kits

Further provided are kits, unit dosages, and articles of manufacture comprising any of the prodrugs described herein. In some embodiments, a kit is provided which contains any one of the prodrug compositions described herein and preferably provides instructions for its use, such as for use in the treatment of the disorders described herein (e.g., tumor).

Kits of the application include one or more containers comprising a prodrug described herein, e.g., for treating a disease. For example, the instructions comprise a description of administration of the prodrug to treat a disease, such as a tumor. The kit may further comprise a description of selecting an individual (e.g., human) suitable for treatment based on identifying whether that individual has the disease and the stage of the disease. The instructions relating to the use of the prodrug generally include information as to dosage, dosing schedule, and route of administration for the intended treatment. The containers may be unit doses, bulk packages (e.g., multi-dose packages) or sub-unit doses. Instructions supplied in the kits of the application are typically written instructions on a label or package insert (e.g., a paper sheet included in the kit), but machine-readable instructions (e.g., instructions carried on a magnetic or optical storage disk) are also acceptable. The kits of the present application are in suitable packaging. Suitable packaging includes, but is not limited to, vials, bottles, jars, flexible packaging (e.g., sealed Mylar or plastic bags), and the like. Also contemplated are packages for use in combination with a specific device, such as an infusion device such as a minipump. A kit may have a sterile access port (for example, the container may be an intravenous solution bag or a vial having a stopper pierceable by a hypodermic injection needle). At least one active agent in the composition is a prodrug as described herein. The container may further comprise a second pharmaceutically active agent. The kits may optionally provide additional components such as buffers and interpretive information. Normally, the kit comprises a container and a label or package insert(s) on or associated with the container.

The present application thus also provides articles of manufacture, which include vials (such as sealed vials), bottles, jars, flexible packaging, and the like. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as a tumor) described herein, and may have a sterile access port (for example, the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples below are intended to be purely exemplary of the application and should therefore not be considered to limit the application in any way. The following examples and detailed descriptions are offered by way of illustration and not by way of limitation.

### Example 1: Generation of Masking Polypeptide (MP)

### Composition of the Masking Polypeptide:

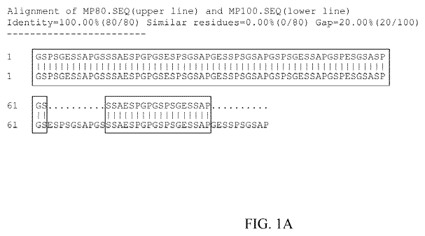

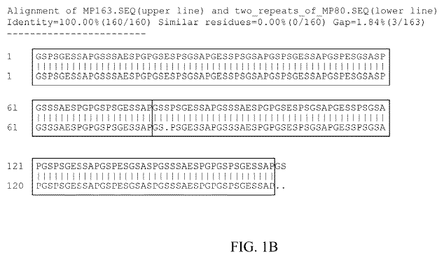

In consideration of choosing the amino acids for a chemically stable and predominantly unstructured masking polypeptide, the masking polypeptide comprises at least 40 contiguous amino acids and is substantially incapable of non-specific binding to serum protein. The final amino acids chosen for masking polypeptide was composed of four types or five types of amino acid residues selected from a group consisting of proline (P), alanine (A), serine (S), glutamic acid (E) and glycine (G). The masking polypeptide sequence was arranged in the ways that there was no single amino acid repeated in three times except serine (S), and the percentage of each amino acid in the masking peptide has a certain ratio: the percentage of amino acid residue A in the masking polypeptide about 5%-20%, the percentage of amino acid residue E in the masking polypeptide is about 1%-20%, the percentage of amino acid residue G in the masking polypeptide is about 15%-30%, the percentage of amino acid residue P in the masking polypeptide is about 15%-40%, the percentage of amino acid residue S in the masking polypeptide is about 20%-40%. The masking polypeptide comprises at least 40 amino acids, the length of the sequence can be extended with the non-repetitive unstructured polypeptides. The exemplary masking polypeptide MP80 was first designed with the sequence SEQ ID NO: 1, the masking polypeptide MP163 and the masking polypeptide MP240 comprise the amino acid sequence of the MP80. Following the rules described above, the masking polypeptides can be designed, not limited to the masking polypeptides shown in Table 1, and tested for the masking activity. FIGS. 1A-1C showed the sequence alignment of MP100 with MP80, MP163 with two repeats of MP80, and MP240 with triple repeats of MP80, respectively. MP80 and MP100 comprised the amino acid sequence of SEQ ID NO: 6, and MP163 and MP240 all comprised the amino acid sequence of MP80.

And all sequences were analyzed with in silicon T cell epitope prediction test, B cell immunogenicity test and with the secondary structure prediction, the masking peptides have no detectable epitope(s) for both T and B cells and no detectable protease cleavage site in masking polypeptide sequences.

TABLE 1

| Masking | SEQ | |
|---|---|---|
| polypeptide (MP) | ID NO. | Sequence |
| MP80 | 1 | GSPSGESSAPGSSSAESPGPGSESPSGSAPGESSPSGSAPG |
| | | SPSGESSAPGSPESGSASPGSSSAESPGPGSPSGESSAP |
| MP100 | 2 | GSPSGESSAPGSSSAESPGPGSESPSGSAPGESSPSGSAPG |
| | | SPSGESSAPGSPESGSASPGSESPSGSAPGSSSAESPGPGS |
| | | PSGESSAPGESSPSGSAP |
| MP96 new | 3 | GPEGPSPEGPESEPGGSPESPEGPSPEGPESPSGPGSGGES |
| | | PGEPSGPEGPESPGSPSGPGGSGPEPSGGESSGEGPSSPE |
| | | GPGSPGGSPEGPSGP |
| MP163 | 4 | GSPSGESSAPGSSSAESPGPGSESPSGSAPGESSPSGSAPG |
| | | SPSGESSAPGSPESGSASPGSSSAESPGPGSPSGESSAPGS |
| | | SPSGESSAPGSSSAESPGPGSESPSGSAPGESSPSGSAPGS |
| | | PSGESSAPGSPESGSASPGSSSAESPGPGSPSGESSAPGS |
| MP240 | 5 | GSPSGESSAPGSSSAESPGPGSESPSGSAPGESSPSGSAPG |
| | | SPSGESSAPGSPESGSASPGSSSAESPGPGSPSGESSAPGS |
| | | PSGESSAPGSSSAESPGPGSESPSGSAPGESSPSGSAPGSP |
| | | SGESSAPGSPESGSASPGSSSAESPGPGSPSGESSAPGSPS |
| | | GESSAPGSSSAESPGPGSESPSGSAPGESSPSGSAPGSPSG |
| | | ESSAPGSPESGSASPGSSSAESPGPGSPSGESSAP |

### Example 2: Expression and Characterization of IL-15 Prodrugs

In order to reduce the toxicity of IL-15 related therapeutic drugs, the IL-15 prodrugs with masking polypeptide were constructed and recombinantly expressed in HEK293 cells. The constructs of IL-15 prodrugs and drugs were shown in Table 2. An exemplary schematic construct illustrating an IL-15 prodrug that includes a masking polypeptide was shown in FIG. 2A. FIG. 2B was an exemplary schematic drawing illustrating the activation process of IL-15 prodrug by released off the masking polypeptide (MP) at the target tissue (e.g., tumor with high levels of MMPs). The prodrugs had very low activities until the masking polypeptide was cleaved off by the protease at the target tissue. The main difference between IL-15 drug and prodrug is that the drug has no masking polypeptide and cleavable moiety (e.g., SB1902-C1, SB1902-C1-variant1, SB1902-C1-variant2 and SB1902-C1-variant3). In the examples, IL-15_L45D, IL-15_L45E, IL-15_Q48K, IL-15_S51D, IL-15_L52D, IL-15_E64K, IL-15_I67D, IL-15_I67E, IL-15_I68D or IL-15_N72D refers to the IL-15 variant comprising mutation L45D, L45E, Q48K, S51D, L52D, E64K, I67D, I67E, I68D or N72D corresponding to human mature wild type IL-15, respectively. Therefore, SB1902-C1-variant3_L45D for example, refers to the IL-15 drug comprising IL-15 L45D variant instead of human mature wild type IL-15 in SB1902-C1-variant3. The non-activatable IL-15 cytokine construct that did not include a cleavable moiety was constructed as control in the following experiments, e.g., SB1902-C4.

TABLE 2

| Construct name | Construct description |
|---|---|
| SB1902-C1 | hIgG Fc(hole)-lk1-IL15 |
| | hIgG Fc(knob)-lk5-IL15Rα_sushi |
| SB1902-C2 | hIgG Fc(hole)-lk1-IL15-lk2-CM1-lk2-MP80 |
| | hIgG Fc(knob)-lk5-IL15Rα_sushi |
| SB1902-C3 | hIgG Fc(hole)-lk1-IL15-lk2-CM2-lk2-MP80 |
| | hIgG Fc(knob)-lk5-IL15Rα_sushi |
| SB1902-C4 | hIgG Fc(hole)-lk1-IL15-lk2-MP80 |
| | hIgG Fc(knob)-lk5-IL15Rα_sushi |
| SB1902-C5 | hIgG Fc(hole)-lk1-IL15-lk2-CM4-lk2-MP80 |
| | hIgG Fc(knob)-lk5-IL15Rα_sushi |
| SB1902-C6 | hIgG Fc(hole)-lk1-IL15-lk2-CM1-lk2-MP96new |
| | hIgG Fc(knob)-lk5-IL15Rα_sushi |
| SB1902-C7 | hIgG Fc(hole)-lk1-IL15-lk2-CM1-lk2-MP100 |
| | hIgG Fc(knob)-lk5-IL15Rα_sushi |
| SB1902-C9 | hIgG Fc(hole)-lk1-IL15-lk2-CM1-lk2-MP163 |
| | hIgG Fc(knob)-lk5-IL15Rα_sushi |
| SB1902-C1-variant1 | hIgG Fc(hole)-lk1-IL15 |
| | hIgG Fc(knob)-lk-IL15Rα_sushi |
| SB1902-C1-variant2 | hIgG Fc(hole)-lk3-IL15Rα_sushi |
| | hIgG Fc(knob)-lk1-IL15 |
| SB1902-C1-variant3 | hIgG Fc(hole-LALA)-lk3-IL15Rα_sushi |
| | hIgG Fc(knob-LALA)-lk1-IL15 |
| SB1902-C1-variant3_L45D | hIgG Fc(hole-LALA)-lk3-IL15Rα_sushi |
| | hIgG Fc(knob-LALA)-lk1-IL15_L45D |
| SB1902-C1-variant3_L45E | hIgG Fc(hole-LALA)-lk3-IL15Rα_sushi |
| | hIgG Fc(knob-LALA)-lk1-IL15_L45E |
| SB1902-C1-variant3_Q48K | hIgG Fc(hole-LALA)-lk3-IL15Rα_sushi |
| | hIgG Fc(knob-LALA)-lk1-IL15_Q48K |
| SB1902-C1-variant3_S51D | hIgG Fc(hole-LALA)-lk3-IL15Rα_sushi |
| | hIgG Fc(knob-LALA)-lk1-IL15_S51D |
| SB1902-C1-variant3_L52D | hIgG Fc(hole-LALA)-lk3-IL15Rα_sushi |
| | hIgG Fc(knob-LALA)-lk1-IL15_L52D |
| SB1902-C1-variant3_E64K | hIgG Fc(hole-LALA)-lk3-IL15Rα_sushi |
| | hIgG Fc(knob-LALA)-lk1-IL15_E64K |
| SB1902-C1-variant3_I67D | hIgG Fc(hole-LALA)-lk3-IL15Rα_sushi |
| | hIgG Fc(knob-LALA)-lk1-IL15_I67D |
| SB1902-C1-variant3_I67E | hIgG Fc(hole-LALA)-lk3-IL15Rα_sushi |
| | hIgG Fc(knob-LALA)-lk1-IL15_I67E |
| SB1902-C1-variant3_I68D | hIgG Fc(hole-LALA)-lk3-IL15Rα_sushi |
| | hIgG Fc(knob-LALA)-lk1-IL15_I68D |
| SB1902-C1-variant3_N72D | hIgG Fc(hole-LALA)-lk3-IL15Rα_sushi |
| | hIgG Fc(knob-LALA)-lk1-IL15_N72D |
| SB1902-C9-variant4 | hIgG Fc(hole-LALA)-lk1-IL15Rα_sushi |
| | hIgG Fc(knob-LALA)-lk1-IL15-lk2-CM1-lk2-MP163 |
| SB1902-C10-variant1 | hIgG Fc(hole)-lk1-IL15-lk2-CM1-lk2-MP240 |
| | hIgG Fc(knob)-lk-IL15Rα_sushi |
| SB1902-C2-variant0 | hIgG Fc(hole)-lk1-IL15-lk2-CM1-lk2-MP80 |
| | hIgG Fc(knob)-lk-IL15Rα_sushi |
| SB1902-C2-variant1 | hIgG Fc(hole)-lk5-IL15Rα_sushi |
| | hIgG Fc(knob)-lk1-IL15-lk2-CM1-lk2-MP80 |
| SB1902-C2-variant2 | hIgG Fc(hole)-lk3-IL15Rα_sushi |
| | hIgG Fc(knob)-lk1-IL15-lk2-CM1-lk2-MP80 |
| SB1902-C9-variant0 | hIgG Fc(hole)-lk-IL15Rα_sushi |
| | hIgG Fc(knob)-lk1-IL15-lk2-CM1-lk2-MP163 |
| SB1902-C9-variant1 | hIgG Fc(hole)-lk5-IL15Rα_sushi |
| | hIgG Fc(knob)-lk1-IL15-lk2-CM1-lk2-MP163 |
| SB1902-C9-variant2 | hIgG Fc(hole)-lk3-IL15Rα_sushi |
| | hIgG Fc(knob)-lk1-IL15-lk2-CM1-lk2-MP163 |
| SB1902-C9-variant2_L45D | hIgG Fc(hole)-lk3-IL15Rα_sushi |
| | hIgG Fc(knob)-lk1-IL15_L45D-lk2-CM1-lk2-MP163 |
| SB1902-C9-variant2_L45E | hIgG Fc(hole)-lk3-IL15Rα_sushi |
| | hIgG Fc(knob)-lk1-IL15_L45E-lk2-CM1-lk2-MP163 |
| SB1902-C9-variant2_Q48K | hIgG Fc(hole)-lk3-IL15Rα_sushi |
| | hIgG Fc(knob)-lk1-IL15_Q48K-lk2-CM1-lk2-MP163 |
| SB1902-C9-variant2_S51D | hIgG Fc(hole)-lk3-IL15Rα_sushi |
| | hIgG Fc(knob)-lk1-IL15_S51D-lk2-CM1-lk2-MP163 |
| SB1902-C9-variant2_L52D | hIgG Fc(hole)-lk3-IL15Rα_sushi |
| | hIgG Fc(knob)-lk1-IL15_L52D-lk2-CM1-lk2-MP163 |
| SB1902-C9-variant2_E64K | hIgG Fc(hole)-lk3-IL15Rα_sushi |
| | hIgG Fc(knob)-lk1-IL15_E64K-lk2-CM1-lk2-MP163 |
| SB1902-C9-variant2_I67D | hIgG Fc(hole)-lk3-IL15Rα_sushi |
| | hIgG Fc(knob)-lk1-IL15_I67D-lk2-CM1-lk2-MP163 |
| SB1902-C9-variant2_I67E | hIgG Fc(hole)-lk3-IL15Rα_sushi |
| | hIgG Fc(knob)-lk1-IL15_I67E-lk2-CM1-lk2-MP163 |
| SB1902-C9-variant2_I68D | hIgG Fc(hole)-lk3-IL15Rα_sushi |
| | hIgG Fc(knob)-lk1-IL15_I68D-lk2-CM1-lk2-MP163 |
| SB1902-C9-variant2_N72D | hIgG Fc(hole)-lk3-IL15Rα_sushi |
| | hIgG Fc(knob)-lk1-IL15_N72D-lk2-CM1-lk2-MP163 |
| SB1902-C9-variant3 | hIgG Fc(hole)-lk1-IL15Rα_sushi |
| | hIgG Fc(knob)-lk1-IL15-lk2-CM1-lk2-MP163 |
| SB1902-C9-variant5 | hIgG Fc(hole-LALA)-lk3-IL15Rα_sushi |
| | hIgG Fc(knob-LALA)-lk1-IL15-lk2-CM1-lk2-MP163 |
| SB1902-C9-variant6 | hIgG Fc(hole)-lk1-IL15-lk2-CM1-lk2-MP163 |
| | hIgG Fc(knob)-lk-IL15Rα_sushi |

Plasmid construction is exemplified with prodrug SB1902-C2 (hIgG Fc(hole)-lk1-IL15-lk2-CM1-lk2-MP80/hIgG Fc(knob)-lk5-IL15Rα_sushi). Similar methods were used for other prodrugs and drugs. Cloning of prodrug expression vector was accomplished with standard molecular techniques. The gene fragments of masking polypeptide (e.g., MP80), human IL-15, human IL-15Rα_sushi was synthesized commercially (Genscripts USA) and digested with restriction enzymes correspondingly. The human IgG1 Fc(hole) or Fc(knob) was PCR amplified and digested with restriction enzymes correspondingly. The cleavable moiety CM1 was synthesized in a single forward and a single reverse nucleotide chain with corresponding restriction enzyme sites in both the 5′ and in the 3′ ends after annealing at 50° C. All synthesized gene fragments and PCR fragments were purified and cloned into plasmid pcDNA3.1 (Invitrogen). After transformation and plating, colonies were picked up and grown at 37° C. overnight in LB media containing carbenicillin. The recombinant plasmids were extracted using commercial kit (Qiagen, Cat number 27104) and sequenced using both T7-forward and BGH reverse primers. The whole coding sequence was verified by DNA sequencing. The exemplary sequences of the cleavable moieties (CM1-CM10) were shown in Table 3, the non-cleavable linkers (lk, lk1, lk2, lk3, and lk5) were shown in Table 4, the exemplary sequences of human wild-type IL-15 (mature form or precursor form), IL-15 variants and IL-15 Rα_sushi (long version from or short version form) were shown in Table 5, the sequences of human IgG1 Fc(hole), Fc(knob), Fc(knob-LALA) and Fc(hole-LALA) were shown in Table 6, and the exemplary sequences of IL-15 prodrug constructs were shown in Table 7. In Table 7, the human IL-15 or IL-15Rα_sushi domain is italicized, the non-cleavable linker is bolded, the cleavable moiety is underlined with single line, the masking polypeptide is underlined with double lines. and the introduced restriction enzyme recognition sites is underlined with dotted line.

For characterization and evaluation, N-terminal 8×His tagged expression constructs of both prodrug and drug were cloned and verified accordingly.

The exemplary cleavage moieties (e.g., CM1, CM2, or CM4) for use in the activatable cytokine of this disclosure could be cleaved by matrix metalloprotease 2 (MMP2), and matrix metalloprotease 9 (MMP9).

TABLE 3

| | SEQ | |
|---|---|---|
| Cleavable moiety | ID NO. | Sequence |
| CM1 | 8 | MVPSALTASG |
| CM2 | 9 | MVLSAVTASG |
| CM4 | 10 | MVVLALTASG |
| CM5 | 11 | MVLSAPTASG |
| CM6 | 12 | MVPSAVTASG |
| CM7 | 13 | MVPSAYTASG |
| CM8 | 14 | MVASALTASG |
| CM9 | 15 | MVVSALTASG |
| CM10 | 16 | MVPSALTVSG |
| CM Composite | 49 | MVX₁X₂AX₃TX₄SG, wherein |
| | | X₁ is selected from P, L, V, or A; |
| | | X₂ is selected from L or S; |
| | | X₃ is selected from L, V, P, or Y; and |
| | | X₄ is selected from A or V |

TABLE 4

| Non-cleavable | SEQ | |
|---|---|---|
| Linker | ID NO. | Sequence |
| lk | 17 | GSG |
| lk1 | 18 | (G4S)1 |
| lk2 | 19 | (G4S)2 |
| lk3 | 20 | (G4S)3 |
| lk5 | 21 | (G4S)5 |

TABLE 5

| | SEQ ID NO. | Sequence |
|---|---|---|
| Human IL15 (mature | 22 | NWVNVISDLKKIEDLIQSMHIDATLYTESDVHPSCKVTA |
| form) | | MKCFLLELQVISLESGDASIHDTVENLIILANNSLSSNGNV |
| | | TESGCKECEELEEKNIKEFLQSFVHIVQMFINTS |
| Human IL-15 | 23 | MRISKPHLRSISIQCYLCLLLNSHFLTEAGIHVFILGCFSAG |
| (precursor form) | | LPKTEANWVNVISDLKKIEDLIQSMHIDATLYTESDVH |
| | | PSCKVTAMKCFLLELQVISLESGDASIHDTVENLIILAN |
| | | NSLSSNGNVTESGCKECEELEEKNIKEFLQSFVHIVQM |
| | | FINTS |
| Human IL-15_L45D | 67 | NWVNVISDLKKIEDLIQSMHIDATLYTESDVHPSCKVTA |
| | | MKCFLDELQVISLESGDASIHDTVENLIILANNSLSSNGNV |
| | | TESGCKECEELEEKNIKEFLQSFVHIVQMFINTS |
| Human IL-15_L45E | 68 | NWVNVISDLKKIEDLIQSMHIDATLYTESDVHPSCKVTA |
| | | MKCFLEELQVISLESGDASIHDTVENLIILANNSLSSNGNV |
| | | TESGCKECEELEEKNIKEFLQSFVHIVQMFINTS |
| Human IL-15_Q48K | 69 | NWVNVISDLKKIEDLIQSMHIDATLYTESDVHPSCKVTA |
| | | MKCFLLELKVISLESGDASIHDTVENLIILANNSLSSNGNV |
| | | TESGCKECEELEEKNIKEFLQSFVHIVQMFINTS |
| Human IL-15_S51D | 70 | NWVNVISDLKKIEDLIQSMHIDATLYTESDVHPSCKVTA |
| | | MKCFLLELQVIDLESGDASIHDTVENLIILANNSLSSNGNV |
| | | TESGCKECEELEEKNIKEFLQSFVHIVQMFINTS |
| Human IL-15_L52D | 71 | NWVNVISDLKKIEDLIQSMHIDATLYTESDVHPSCKVTA |
| | | MKCFLLELQVISDESGDASIHDTVENLIILANNSLSSNGNV |
| | | TESGCKECEELEEKNIKEFLQSFVHIVQMFINTS |
| Human IL-15_N72D | 72 | NWVNVISDLKKIEDLIQSMHIDATLYTESDVHPSCKVTA |
| | | MKCFLLELQVISLESGDASIHDTVENLIILANDSLSSNGNV |
| | | TESGCKECEELEEKNIKEFLQSFVHIVQMFINTS |
| Human IL-15_E64K | 73 | NWVNVISDLKKIEDLIQSMHIDATLYTESDVHPSCKVTA |
| | | MKCFLLELQVIQLESGDASIHDTVKNLIILANNSLSSNGN |
| | | VTESGCKECEELEEKNIKEFLQSFVHIVQMFINTS |
| Human IL-15_I67D | 74 | NWVNVISDLKKIEDLIQSMHIDATLYTESDVHPSCKVTA |
| | | MKCFLLELQVIQLESGDASIHDTVENLDILANNSLSSNGN |
| | | VTESGCKECEELEEKNIKEFLQSFVHIVQMFINTS |
| Human IL-15_I67E | 75 | NWVNVISDLKKIEDLIQSMHIDATLYTESDVHPSCKVTA |
| | | MKCFLLELQVIQLESGDASIHDTVENLEILANNSLSSNGN |
| | | VTESGCKECEELEEKNIKEFLQSFVHIVQMFINTS |
| Human IL-15_I68D | 76 | NWVNVISDLKKIEDLIQSMHIDATLYTESDVHPSCKVTA |
| | | MKCFLLELQVIQLESGDASIHDTVENLIDLANNSLSSNGN |
| | | VTESGCKECEELEEKNIKEFLQSFVHIVQMFINTS |
| Human IL15Rα_sushi | 24 | ITCPPPMSVEHADIWVKSYSLYSRERYICNSGFKRKAGTS |
| (short version 1) | | SLTECVLNKATNVAHWTTPSLKCIR |
| Human IL15Rα_sushi | 25 | TCPPPMSVEHADIWVKSYSLYSRERYICNSGFKRKAGTSS |
| (short version 2) | | LTECVLNKATNVAHWTTPSLKCIR |
| Human IL15Rα_sushi | 26 | ITCPPPMSVEHADIWVKSYSLYSRERYICNSGFKRKAGTS |
| (long version) | | SLTECVLNKATNVAHWTTPSLKCIRDPALVHQRPAPP |

TABLE 6

| | SEQ ID NO. | Sequence |
|---|---|---|
| hIgG1 Fc(hole) | 27 | DKTHTCPPCPAPELLGGPSVFLFPPKPKDTLMISRTPEVTCV |
| T366S + L368A + | | VVDVSHEDPEVKFNWYVDGVEVHNAKTKPREEQYNSTY |
| Y407V | | RVVSVLTVLHQDWLNGKEYKCKVSNKALPAPIEKTISKAK |
| | | GQPREPQVYTLPPSREEMTKNQVSLSCAVKGFYPSDIAVE |
| | | WESNGQPENNYKTTPPVLDSDGSFFLVSKLTVDKSRWQQ |
| | | GNVFSCSVMHEALHNHYTQKSLSLSPGK |
| hIgG1 Fc(knob) | 28 | DKTHTCPPCPAPELLGGPSVFLFPPKPKDTLMISRTPEVTCV |
| T366W | | VVDVSHEDPEVKFNWYVDGVEVHNAKTKPREEQYNSTY |
| | | RVVSVLTVLHQDWLNGKEYKCKVSNKALPAPIEKTISKAK |
| | | GQPREPQVYTLPPSREEMTKNQVSLWCLVKGFYPSDIAVE |
| | | WESNGQPENNYKTTPPVLDSDGSFFLYSKLTVDKSRWQQ |
| | | GNVFSCSVMHEALHNHYTQKSLSLSPGK |
| hIgG1 Fc(hole- | 29 | DKTHTCPPCPAPEAAGGPSVFLFPPKPKDTLMISRTPEVTC |
| LALA) | | VVVDVSHEDPEVKFNWYVDGVEVHNAKTKPREEQYNST |
| | | YRVVSVLTVLHQDWLNGKEYKCKVSNKALPAPIEKTISKA |
| | | KGQPREPQVYTLPPSREEMTKNQVSLSCAVKGFYPSDIAV |
| | | EWESNGQPENNYKTTPPVLDSDGSFFLVSKLTVDKSRWQQ |
| | | GNVFSCSVMHEALHNHYTQKSLSLSPGK |
| hIgG1 Fc(knob- | 30 | DKTHTCPPCPAPEAAGGPSVFLFPPKPKDTLMISRTPEVTC |
| LALA) | | VVVDVSHEDPEVKFNWYVDGVEVHNAKTKPREEQYNST |
| | | YRVVSVLTVLHQDWLNGKEYKCKVSNKALPAPIEKTISKA |
| | | KGQPREPQVYTLPPSREEMTKNQVSLWCLVKGFYPSDIAV |
| | | EWESNGQPENNYKTTPPVLDSDGSFFLYSKLTVDKSRWQQ |
| | | GNVFSCSVMHEALHNHYTQKSLSLSPGK |

TABLE 7

| | | SEQ | |
|---|---|---|---|
| Construct | Construct | ID | |
| name | description | NO. | Sequence |
| SB1902-C1 | hIgG Fc(hole)-lk1- | 31 | DKTHTCPPCPAPELLGGPSVFLFPPKPKDTLMISRT |
| | IL15 | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 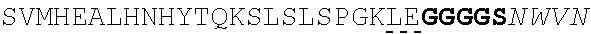 |
| | | | VISDLKKIEDLIQSMHIDATLYTESDVHPSCKVTAMKC |
| | | | FLLELQVISLESGDASIHDTVENLIILANNSLSSNGNVT |
| | | | ESGCKECEELEEKNIKEFLQSFVHIVOMFINTS |
| | hIgG Fc(knob)-lk5- | 32 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 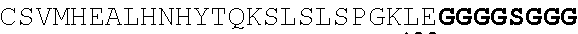 |
| | | | 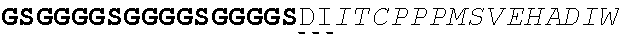 |
| | | | VKSYSLYSRERYICNSGFKRKAGTSSLTECVLNKATNV |
| | | | AHWTTPSLKCIR |
| SB1902-C2 | hIgG Fc(hole)-lk1- | 33 | DKTHTCPPCPAPELLGGPSVFLFPPKPKDTLMISRT |
| | IL15-lk2-CM1-lk2- | | PEVTCVVVDVSHEDPEVKFNWYVDGVEVHNAKT |
| | MP80 | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 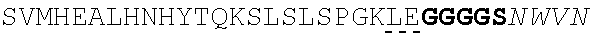 |
| | | | VISDLKKIEDLIQSMHIDATLYTESDVHPSCKVTAMKC |
| | | | FLLELQVISLESGDASIHDTVENLIILANNSLSSNGNVT |
| | | | 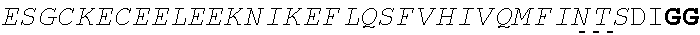 |
| | | | GGSGGGGSMVPSALTASGGGGGSGGGGSRGSPS |
| | | | GESSAPGSSSAESPGPGSESPSGSAPGESSPSGSAPG |
| | | | SPSGESSAPGSPESGSASPGSSSAESPGPGSPSGESS |
| | | | AP |
| | hIgG Fc(knob)-lk5- | 32 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 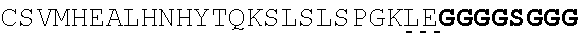 |
| | | | 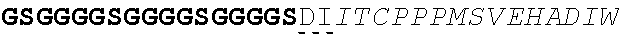 |
| | | | VKSYSLYSRERYICNSGFKRKAGTSSLTECVLNKATNV |
| | | | AHWTTPSLKCIR |
| SB1902-C3 | hIgG Fc(hole)-lk1- | 34 | DKTHTCPPCPAPELLGGPSVFLFPPKPKDTLMISRT |
| | IL15-lk2-CM2-lk2- | | PEVTCVVVDVSHEDPEVKFNWYVDGVEVHNAKT |
| | MP80 | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 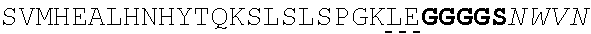 |
| | | | VISDLKKIEDLIQSMHIDATLYTESDVHPSCKVTAMKC |
| | | | FLLELQVISLESGDASIHDTVENLIILANNSLSSNGNVT |
| | | | 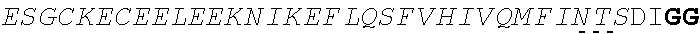 |
| | | | 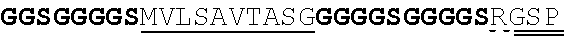 |
| | | | SGESSAPGSSSAESPGPGSESPSGSAPGESSPSGSAP |
| | | | GSPSGESSAPGSPESGSASPGSSSAESPGPGSPSGES |
| | | | SAP |
| | hIgG Fc(knob)-lk5- | 32 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 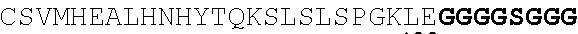 |
| | | | 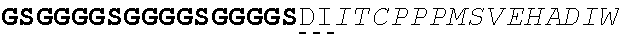 |
| | | | VKSYSLYSRERYICNSGFKRKAGTSSLTECVLNKATNV |
| | | | AHWTTPSLKCIR |
| SB1902-C4 | hIgG Fc(hole)-lk1- | 35 | DKTHTCPPCPAPELLGGPSVFLFPPKPKDTLMISRT |
| | IL15-lk2-MP80 | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 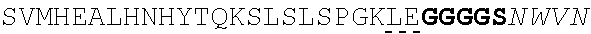 |
| | | | VISDLKKIEDLIQSMHIDATLYTESDVHPSCKVTAMKC |
| | | | FLLELQVISLESGDASIHDTVENLIILANNSLSSNGNVT |
| | | | 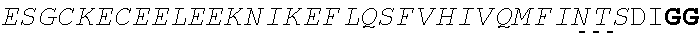 |
| | | | GGSGGGGSRGSPSGESSAPGSSSAESPGPGSESPS |
| | | | GSAPGESSPSGSAPGSPSGESSAPGSPESGSASPGSS |
| | | | SAESPGPGSPSGESSAP |
| | hIgG Fc(knob)-lk5- | 32 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 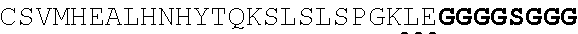 |
| | | | 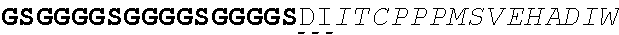 |
| | | | VKSYSLYSRERYICNSGFKRKAGTSSLTECVLNKATNV |
| | | | AHWTTPSLKCIR |
| SB1902-C5 | hIgG Fc(hole)-lk1- | 36 | DKTHTCPPCPAPELLGGPSVFLFPPKPKDTLMISRT |
| | IL15-lk2-CM4-lk2- | | PEVTCVVVDVSHEDPEVKFNWYVDGVEVHNAKT |
| | MP80 | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 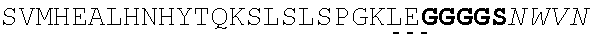 |
| | | | VISDLKKIEDLIQSMHIDATLYTESDVHPSCKVTAMKC |
| | | | FLLELQVISLESGDASIHDTVENLIILANNSLSSNGNVT |
| | | | 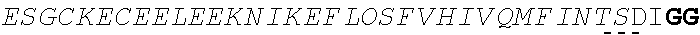 |
| | | | 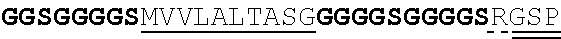 |
| | | | SGESSAPGSSSAESPGPGSESPSGSAPGESSPSGSAP |
| | | | GSPSGESSAPGSPESGSASPGSSSAESPGPGSPSGES |
| | | | SAP |
| | hIgG Fc(knob)-lk5- | 32 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 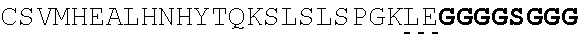 |
| | | | 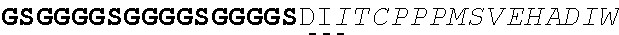 |
| | | | VKSYSLYSRERYICNSGFKRKAGTSSLTECVLNKATNV |
| | | | AHWTTPSLKCIR |
| SB1902-C6 | hIgG Fc(hole)-lk1- | 37 | DKTHTCPPCPAPELLGGPSVFLFPPKPKDTLMISRT |
| | IL15-lk2-CM1-lk2- | | PEVTCVVVDVSHEDPEVKFNWYVDGVEVHNAKT |
| | MP96new | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | SVMHEALHNHYTQKSLSLSPGKLEGGGGSNWVN |
| | | | VISDLKKIEDLIQSMHIDATLYTESDVHPSCKVTAMKC |
| | | | FLLELQVISLESGDASIHDTVENLIILANNSLSSNGNVT |
| | | | 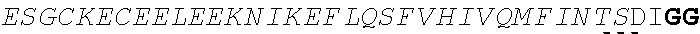 |
| | | | GGSGGGGSMVPSALTASGGGGGSGGGGSRGPE |
| | | | GPSPEGPESEPGGSPESPEGPSPEGPESPSGPGSGGE |
| | | | SPGEPSGPEGPESPGSPSGPGGSGPEPSGGESSGEG |
| | | | PSSPEGPGSPGGSPEGPSGP |
| | hIgG Fc(knob)-lk5- | 32 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 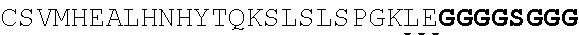 |
| | | | 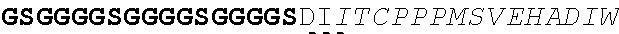 |
| | | | VKSYSLYSRERYICNSGFKRKAGTSSLTECVLNKATNV |
| | | | AHWTTPSLKCIR |
| SB1902-C7 | hIgG Fc(hole)-lk1- | 38 | DKTHTCPPCPAPELLGGPSVFLFPPKPKDTLMISRT |
| | IL15-lk2-CM1-lk2- | | PEVTCVVVDVSHEDPEVKFNWYVDGVEVHNAKT |
| | MP100 | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 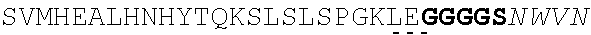 |
| | | | VISDLKKIEDLIQSMHIDATLYTESDVHPSCKVTAMKC |
| | | | FLLELQVISLESGDASIHDTVENLIILANNSLSSNGNVT |
| | | | 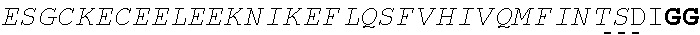 |
| | | | 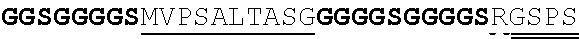 |
| | | | GESSAPGSSSAESPGPGSESPSGSAPGESSPSGSAPG |
| | | | SPSGESSAPGSPESGSASPGSESPSGSAPGSSSAESP |
| | | | GPGSPSGESSAPGESSPSGSAP |
| | hIgG Fc(knob)-lk5- | 32 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 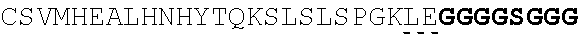 |
| | | | 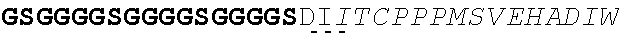 |
| | | | VKSYSLYSRERYICNSGFKRKAGTSSLTECVLNKATNV |
| | | | AHWTTPSLKCIR |
| SB1902-C9 | hIgG Fc(hole)-lk1- | 39 | DKTHTCPPCPAPELLGGPSVFLFPPKPKDTLMISRT |
| | IL15-lk2-CM1-lk2- | | PEVTCVVVDVSHEDPEVKFNWYVDGVEVHNAKT |
| | MP163 | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 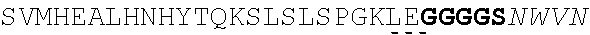 |
| | | | VISDLKKIEDLIQSMHIDATLYTESDVHPSCKVTAMKC |
| | | | FLLELQVISLESGDASIHDTVENLIILANNSLSSNGNVT |
| | | | 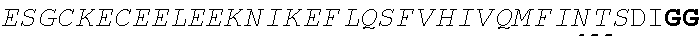 |
| | | | 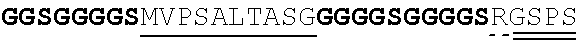 |
| | | | GESSAPGSSSAESPGPGSESPSGSAPGESSPSGSAPG |
| | | | SPSGESSAPGSPESGSASPGSSSAESPGPGSPSGESS |
| | | | APGSSPSGESSAPGSSSAESPGPGSESPSGSAPGESS |
| | | | PSGSAPGSPSGESSAPGSPESGSASPGSSSAESPGPG |
| | | | SPSGESSAPGS |
| | hIgG Fc(knob)-lk5- | 32 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 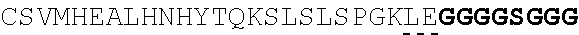 |
| | | | 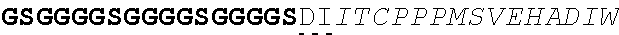 |
| | | | VKSYSLYSRERYICNSGFKRKAGTSSLTECVLNKATNV |
| | | | AHWTTPSLKCIR |
| SB1902-C1- | hIgG Fc(hole)-lk1- | 31 | DKTHTCPPCPAPELLGGPSVFLFPPKPKDTLMISRT |
| variant1 | IL15 | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 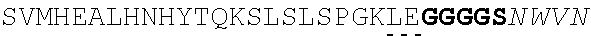 |
| | | | VISDLKKIEDLIQSMHIDATLYTESDVHPSCKVTAMKC |
| | | | FLLELQVISLESGDASIHDTVENLIILANNSLSSNGNVT |
| | | | ESGCKECEELEEKNIKEFLQSFVHIVQMFINTS |
| | hIgG Fc(knob)-lk- | 40 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | CSVMHEALHNHYTQKSLSLSPGKGSGTCPPPMSV |
| | | | EHADIWVKSYSLYSRERYICNSGFKRKAGTSSLTECVL |
| | | | NKATNVAHWTTPSLKCIR |
| SB1902-C1- | hIgG Fc(hole)-lk3- | 41 | DKTHTCPPCPAPELLGGPSVFLFPPKPKDTLMISRT |
| variant2 | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 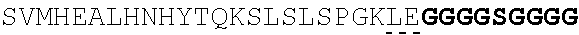 |
| | | | 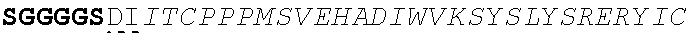 |
| | | | NSGFKRKAGTSSLTECVLNKATNVAHWTTPSLKCIR |
| | hIgG Fc(knob)-lk1- | 42 | DKTHTCPPCPAPELLGGPSVFLFPPKPKDTLMISRT |
| | IL15 | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 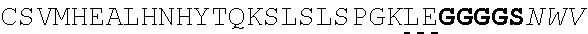 |
| | | | NVISDLKKIEDLIQSMHIDATLYTESDVHPSCKVTAMK |
| | | | CFLLELQVISLESGDASIHDTVENLIILANNSLSSNGNV |
| | | | TESGCKECEELEEKNIKEFLQSFVHIVQMFINTS |
| SB1902-C1- | hIgG Fc(hole- | 43 | DKTHTCPPCPAPEaaGGPSVFLFPPKPKDTLMISRT |
| variant3 | LALA)-lk3- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 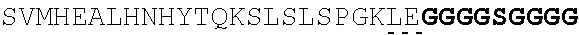 |
| | | | 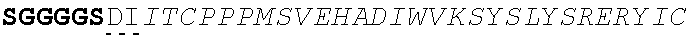 |
| | | | NSGFKRKAGTSSLTECVLNKATNVAHWTTPSLKCIR |
| | hIgG Fc(knob- | 44 | DKTHTCPPCPAPEaaGGPSVFLFPPKPKDTLMISRT |
| | LALA)-lk1-IL15 | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 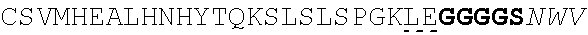 |
| | | | NVISDLKKIEDLIQSMHIDATLYTESDVHPSCKVTAMK |
| | | | CFLLELQVISLESGDASIHDTVENLIILANNSLSSNGNV |
| | | | TESGCKECEELEEKNIKEFLQSFVHIVQMFINTS |
| SB1902-C1- | hIgG Fc(hole- | 43 | DKTHTCPPCPAPEaaGGPSVFLFPPKPKDTLMISRT |
| variant3_L45D | LALA)-lk3- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 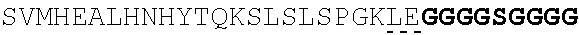 |
| | | | 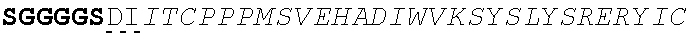 |
| | | | NSGFKRKAGTSSLTECVLNKATNVAHWTTPSLKCIR |
| | hIgG Fc(knob- | 77 | DKTHTCPPCPAPEaaGGPSVFLFPPKPKDTLMISRT |
| | LALA)-lk1- | | PEVTCVVVDVSHEDPEVKFNWYVDGVEVHNAKT |
| | IL15_L45D | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 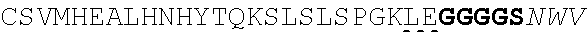 |
| | | | NVISDLKKIEDLIQSMHIDATLYTESDVHPSCKVTAMK |
| | | | CFLDELQVISLESGDASIHDTVENLIILANNSLSSNGNV |
| | | | TESGCKECEELEEKNIKEFLQSFVHIVQMFINTS |
| SB1902-C1- | hIgG Fc(hole- | 43 | DKTHTCPPCPAPEaaGGPSVFLFPPKPKDTLMISRT |
| variant3_L45E | LALA)-lk3- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 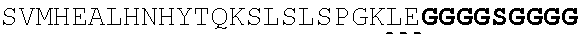 |
| | | | 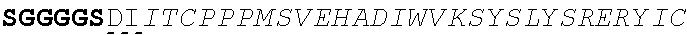 |
| | | | NSGFKRKAGTSSLTECVLNKATNVAHWTTPSLKCIR |
| | hIgG Fc(knob- | 78 | DKTHTCPPCPAPEaaGGPSVFLFPPKPKDTLMISRT |
| | LALA)-lk1- | | PEVTCVVVDVSHEDPEVKFNWYVDGVEVHNAKT |
| | IL15_L45E | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 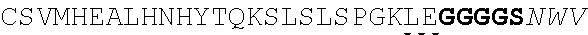 |
| | | | NVISDLKKIEDLIQSMHIDATLYTESDVHPSCKVTAMK |
| | | | CFLEELQVISLESGDASIHDTVENLIILANNSLSSNGNV |
| | | | TESGCKECEELEEKNIKEFLQSFVHIVQMFINTS |
| SB1902-C1- | hIgG Fc(hole- | 43 | DKTHTCPPCPAPEaaGGPSVFLFPPKPKDTLMISRT |
| variant3_Q48K | LALA)-lk3- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 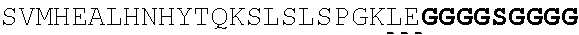 |
| | | | 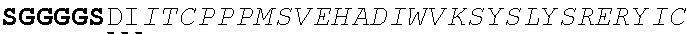 |
| | | | NSGFKRKAGTSSLTECVLNKATNVAHWTTPSLKCIR |
| | hIgG Fc(knob- | 79 | DKTHTCPPCPAPEaaGGPSVFLFPPKPKDTLMISRT |
| | LALA)-lk1- | | PEVTCVVVDVSHEDPEVKFNWYVDGVEVHNAKT |
| | IL15_Q48K | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 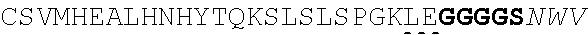 |
| | | | NVISDLKKIEDLIQSMHIDATLYTESDVHPSCKVTAMK |
| | | | CFLLELKVISLESGDASIHDTVENLIILANNSLSSNGNV |
| | | | TESGCKECEELEEKNIKEFLQSFVHIVQMFINTS |
| SB1902-C1- | hIgG Fc(hole- | 43 | DKTHTCPPCPAPEaaGGPSVFLFPPKPKDTLMISRT |
| variant3_S51D | LALA)-lk3- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 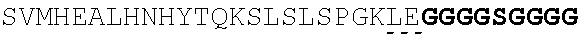 |
| | | | 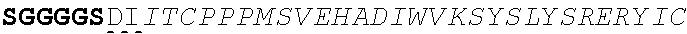 |
| | | | NSGFKRKAGTSSLTECVLNKATNVAHWTTPSLKCIR |
| | hIgG Fc(knob- | 80 | DKTHTCPPCPAPEaaGGPSVFLFPPKPKDTLMISRT |
| | LALA)-lk1- | | PEVTCVVVDVSHEDPEVKFNWYVDGVEVHNAKT |
| | IL15_S51D | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 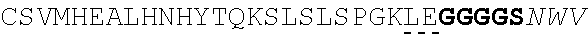 |
| | | | NVISDLKKIEDLIQSMHIDATLYTESDVHPSCKVTAMK |
| | | | CFLLELQVIDLESGDASIHDTVENLIILANNSLSSNGNV |
| | | | TESGCKECEELEEKNIKEFLQSFVHIVOMFINTS |
| SB1902-C1- | hIgG Fc(hole- | 43 | DKTHTCPPCPAPEaaGGPSVFLFPPKPKDTLMISRT |
| variant3_L52D | LALA)-lk3- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 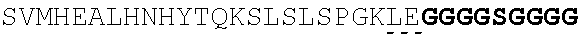 |
| | | | 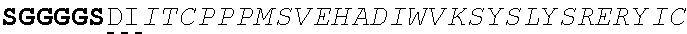 |
| | | | NSGFKRKAGTSSLTECVLNKATNVAHWTTPSLKCIR |
| | hIgG Fc(knob- | 81 | DKTHTCPPCPAPEaaGGPSVFLFPPKPKDTLMISRT |
| | LALA)-lk1- | | PEVTCVVVDVSHEDPEVKFNWYVDGVEVHNAKT |
| | IL15_L52D | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 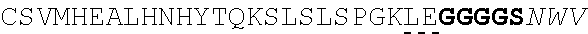 |
| | | | NVISDLKKIEDLIQSMHIDATLYTESDVHPSCKVTAMK |
| | | | CFLLELQVISDESGDASIHDTVENLIILANNSLSSNGNV |
| | | | TESGCKECEELEEKNIKEFLQSFVHIVQMFINTS |
| SB1902-C1- | hIgG Fc(hole- | 43 | DKTHTCPPCPAPEaaGGPSVFLFPPKPKDTLMISRT |
| variant3_E64K | LALA)-lk3- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 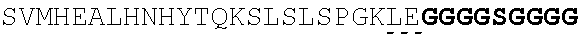 |
| | | | 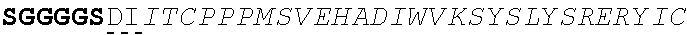 |
| | | | NSGFKRKAGTSSLTECVLNKATNVAHWTTPSLKCIR |
| | hIgG Fc(knob- | 82 | DKTHTCPPCPAPEaaGGPSVFLFPPKPKDTLMISRT |
| | LALA)-lk1- | | PEVTCVVVDVSHEDPEVKFNWYVDGVEVHNAKT |
| | IL15_E64K | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 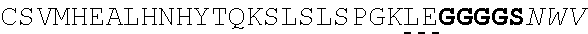 |
| | | | NVISDLKKIEDLIQSMHIDATLYTESDVHPSCKVTAMK |
| | | | CFLLELQVISLESGDASIHDTVKNLIILANNSLSSNGNV |
| | | | TESGCKECEELEEKNIKEFLQSFVHIVQMFINTS |
| SB1902-C1- | hIgG Fc(hole- | 43 | DKTHTCPPCPAPEaaGGPSVFLFPPKPKDTLMISRT |
| variant3_I67D | LALA)-lk3- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 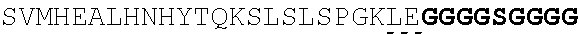 |
| | | | 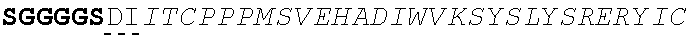 |
| | | | NSGFKRKAGTSSLTECVLNKATNVAHWTTPSLKCIR |
| | hIgG Fc(knob- | 83 | DKTHTCPPCPAPEaaGGPSVFLFPPKPKDTLMISRT |
| | LALA)-lk1- | | PEVTCVVVDVSHEDPEVKFNWYVDGVEVHNAKT |
| | IL15_I67D | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 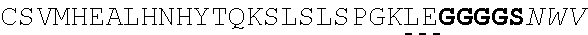 |
| | | | NVISDLKKIEDLIQSMHIDATLYTESDVHPSCKVTAMK |
| | | | CFLLELQVISLESGDASIHDTVENLDILANNSLSSNGN |
| | | | VTESGCKECEELEEKNIKEFLQSFVHIVQMFINTS |
| SB1902-C1- | hIgG Fc(hole- | 43 | DKTHTCPPCPAPEaaGGPSVFLFPPKPKDTLMISRT |
| variant3_I67E | LALA)-lk3- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 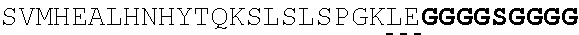 |
| | | | 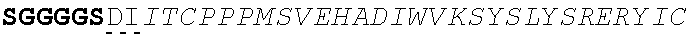 |
| | | | NSGFKRKAGTSSLTECVLNKATNVAHWTTPSLKCIR |
| | hIgG Fc(knob- | 84 | DKTHTCPPCPAPEaaGGPSVFLFPPKPKDTLMISRT |
| | LALA)-lk1- | | PEVTCVVVDVSHEDPEVKFNWYVDGVEVHNAKT |
| | IL15_I67E | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 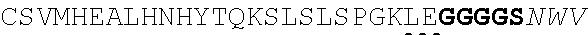 |
| | | | NVISDLKKIEDLIQSMHIDATLYTESDVHPSCKVTAMK |
| | | | CFLLELQVISLESGDASIHDTVENLEILANNSLSSNGN |
| | | | VTESGCKECEELEEKNIKEFLQSFVHIVQMFINTS |
| SB1902-C1- | hIgG Fc(hole- | 43 | DKTHTCPPCPAPEaaGGPSVFLFPPKPKDTLMISRT |
| variant3_I68D | LALA)lk3- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 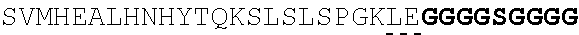 |
| | | | 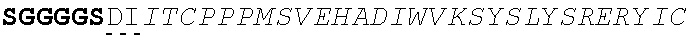 |
| | | | NSGFKRKAGTSSLTECVLNKATNVAHWTTPSLKCIR |
| | hIgG Fc(knob- | 85 | DKTHTCPPCPAPEaaGGPSVFLFPPKPKDTLMISRT |
| | LALA)-lk1- | | PEVTCVVVDVSHEDPEVKFNWYVDGVEVHNAKT |
| | IL15_I68D | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 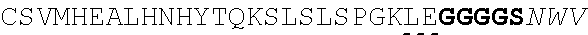 |
| | | | NVISDLKKIEDLIQSMHIDATLYTESDVHPSCKVTAMK |
| | | | CFLLELQVISLESGDASIHDTVENLIDLANNSLSSNGN |
| | | | VTESGCKECEELEEKNIKEFLQSFVHIVQMFINTS |
| SB1902-C1- | hIgG Fc(hole- | 43 | DKTHTCPPCPAPEaaGGPSVFLFPPKPKDTLMISRT |
| variant3_N72D | LALA)-lk3- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 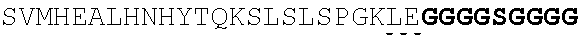 |
| | | | 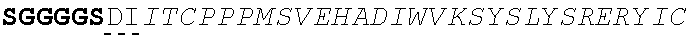 |
| | | | NSGFKRKAGTSSLTECVLNKATNVAHWTTPSLKCIR |
| | hIgG Fc(knob- | 86 | DKTHTCPPCPAPEaaGGPSVFLFPPKPKDTLMISRT |
| | LALA)-lkl- | | PEVTCVVVDVSHEDPEVKFNWYVDGVEVHNAKT |
| | IL15_N72D | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 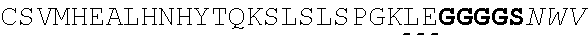 |
| | | | NVISDLKKIEDLIQSMHIDATLYTESDVHPSCKVTAMK |
| | | | CFLLELQVISLESGDASIHDTVENLIILANDSLSSNGNV |
| | | | TESGCKECEELEEKNIKEFLQSFVHIVQMFINTS |
| SB1902-C9- | hIgG Fc(hole- | 45 | DKTHTCPPCPAPEaaGGPSVFLFPPKPKDTLMISRT |
| variant4 | LALA)-lk1- | | PEVTCVVVDVSHEDPEVKFNWYVDGVEVHNAKT |
| | IL15Rα_sushi | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 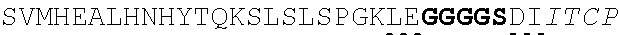 |
| | | | PPMSVEHADIWVKSYSLYSRERYICNSGFKRKAGTSSL |
| | | | TECVLNKATNVAHWTTPSLKCIR |
| | hIgG Fc(knob- | 46 | DKTHTCPPCPAPEaaGGPSVFLFPPKPKDTLMISRT |
| | LALA)-lk1-IL15- | | PEVTCVVVDVSHEDPEVKFNWYVDGVEVHNAKT |
| | lk2-CM1-lk2- | | KPREEQYNSTYRVVSVLTVLHQDWLNGKEYKCK |
| | MP163 | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 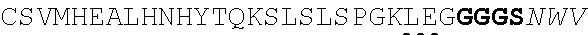 |
| | | | NVISDLKKIEDLIQSMHIDATLYTESDVHPSCKVTAMK |
| | | | CFLLELQVISLESGDASIHDTVENLIILANNSLSSNGNV |
| | | | 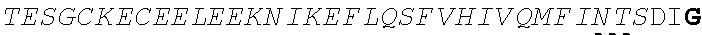 |
| | | | 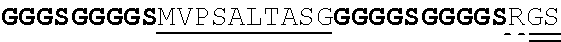 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| SB1902-C10- | hIgG Fc(hole)-lk1- | 47 | DKTHTCPPCPAPELLGGPSVFLFPPKPKDTLMISRT |
| variant1 | IL15-lk2-CM1-lk2- | | PEVTCVVVDVSHEDPEVKFNWYVDGVEVHNAKT |
| | MP240 | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 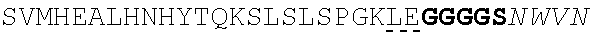 |
| | | | VISDLKKIEDLIQSMHIDATLYTESDVHPSCKVTAMKC |
| | | | FLLELQVISLESGDASIHDTVENLIILANNSLSSNGNVT |
| | | | 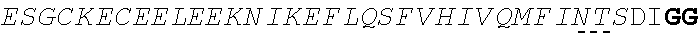 |
| | | | 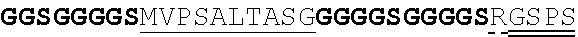 |
| | | | GESSAPGSSSAESPGPGSESPSGSAPGESSPSGSAPG |
| | | | SPSGESSAPGSPESGSASPGSSSAESPGPGSPSGESS |
| | | | APGSPSGESSAPGSSSAESPGPGSESPSGSAPGESSP |
| | | | SGSAPGSPSGESSAPGSPESGSASPGSSSAESPGPGS |
| | | | PSGESSAPGSPSGESSAPGSSSAESPGPGSESPSGSA |
| | | | PGESSPSGSAPGSPSGESSAPGSPESGSASPGSSSAE |
| | | | SPGPGSPSGESSAP |
| | hIgG Fc(knob)-lk- | 40 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | CSVMHEALHNHYTQKSLSLSPGKGSGTCPPPMSV |
| | | | EHADIWVKSYSLYSRERYICNSGFKRKAGTSSLTECVL |
| | | | NKATNVAHWTTPSLKCIR |
| SB1902-C2- | hIgG Fc(hole)-lk1- | 33 | DKTHTCPPCPAPELLGGPSVFLFPPKPKDTLMISRT |
| variant0 | IL15-lk2-CM1-lk2- | | PEVTCVVVDVSHEDPEVKFNWYVDGVEVHNAKT |
| | MP80 | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 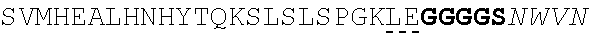 |
| | | | VISDLKKIEDLIQSMHIDATLYTESDVHPSCKVTAMKC |
| | | | FLLELQVISLESGDASIHDTVENLIILANNSLSSNGNVT |
| | | | 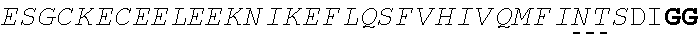 |
| | | | 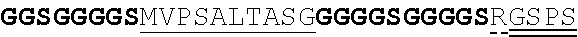 |
| | | | GESSAPGSSSAESPGPGSESPSGSAPGESSPSGSAPG |
| | | | SPSGESSAPGSPESGSASPGSSSAESPGPGSPSGESS |
| | | | AP |
| | hIgG Fc(knob)-lk- | 40 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | CSVMHEALHNHYTQKSLSLSPGKGSGTCPPPMSV |
| | | | EHADIWVKSYSLYSRERYICNSGFKRKAGTSSLTECVL |
| | | | NKATNVAHWTTPSLKCIR |
| SB1902-C2- | hIgG Fc(hole)-lk5- | 62 | DKTHTCPPCPAPELLGGPSVFLFPPKPKDTLMISRT |
| variant1 | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 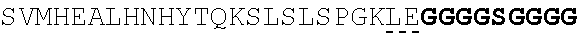 |
| | | | 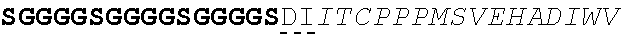 |
| | | | KSYSLYSRERYICNSGFKRKAGTSSLTECVLNKATNVA |
| | | | HWTTPSLKCIR |
| | hIgG Fc(knob)-lk1- | 63 | DKTHTCPPCPAPELLGGPSVFLFPPKPKDTLMISRT |
| | IL15-1k2-CM1-lk2- | | PEVTCVVVDVSHEDPEVKFNWYVDGVEVHNAKT |
| | MP80 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 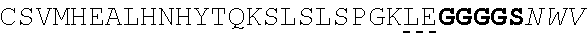 |
| | | | NVISDLKKIEDLIQSMHIDATLYTESDVHPSCKVTAMK |
| | | | CFLLELQVISLESGDASIHDTVENLIILANNSLSSNGNV |
| | | | 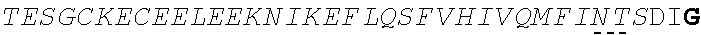 |
| | | | 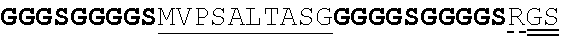 |
| | | | PSGESSAPGSSSAESPGPGSESPSGSAPGESSPSGSA |
| | | | PGSPSGESSAPGSPESGSASPGSSSAESPGPGSPSGE |
| | | | SSAP |
| SB1902-C2- | hIgG Fc(hole)-lk3- | 41 | DKTHTCPPCPAPELLGGPSVFLFPPKPKDTLMISRT |
| variant2 | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 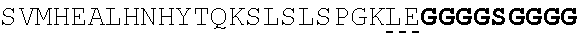 |
| | | | 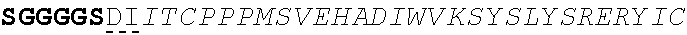 |
| | | | NSGFKRKAGTSSLTECVLNKATNVAHWTTPSLKCIR |
| | hIgG Fc(knob)-lk1- | 63 | DKTHTCPPCPAPELLGGPSVFLFPPKPKDTLMISRT |
| | IL15-lk2-CM1-lk2- | | PEVTCVVVDVSHEDPEVKFNWYVDGVEVHNAKT |
| | MP80 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 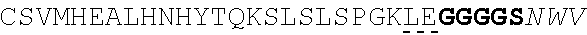 |
| | | | NVISDLKKIEDLIQSMHIDATLYTESDVHPSCKVTAMK |
| | | | CFLLELQVISLESGDASIHDTVENLIILANNSLSSNGNV |
| | | | 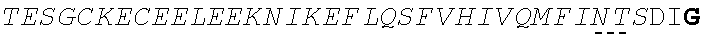 |
| | | | 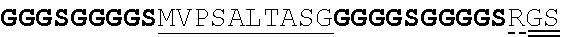 |
| | | | PSGESSAPGSSSAESPGPGSESPSGSAPGESSPSGSA |
| | | | PGSPSGESSAPGSPESGSASPGSSSAESPGPGSPSGE |
| | | | SSAP |
| SB1902-C9- | hIgG Fc(hole)-lk- | 64 | DKTHTCPPCPAPELLGGPSVFLFPPKPKDTLMISRT |
| variant0 | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | SVMHEALHNHYTQKSLSLSPGKGSGTCPPPMSVE |
| | | | HADIWVKSYSLYSRERYICNSGFKRKAGTSSLTECVLN |
| | | | KATNVAHWTTPSLKCIR |
| | hIgG Fc(knob)-lk1- | 65 | DKTHTCPPCPAPELLGGPSVFLFPPKPKDTLMISRT |
| | IL15-lk2-CM1-lk2- | | PEVTCVVVDVSHEDPEVKFNWYVDGVEVHNAKT |
| | MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 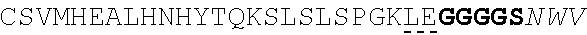 |
| | | | NVISDLKKIEDLIQSMHIDATLYTESDVHPSCKVTAMK |
| | | | CFLLELQVISLESGDASIHDTVENLIILANNSLSSNGNV |
| | | | 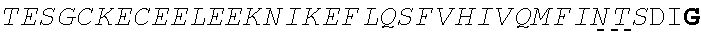 |
| | | | 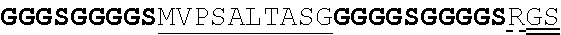 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| | hIgG Fc(hole)-lk5- | 62 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 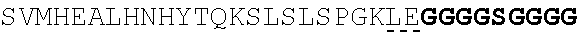 |
| | | | 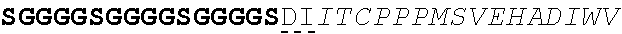 |
| | | | KSYSLYSRERYICNSGFKRKAGTSSLTECVLNKATNVA |
| | | | HWTTPSLKCIR |
| SB1902-C9- | hIgG Fc(knob)-lk1- | 65 | DKTHTCPPCPAPELLGGPSVFLFPPKPKDTLMISRT |
| variant1 | IL15-lk2-CM1-lk2- | | PEVTCVVVDVSHEDPEVKFNWYVDGVEVHNAKT |
| | MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 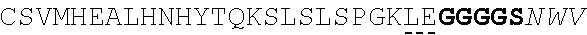 |
| | | | NVISDLKKIEDLIQSMHIDATLYTESDVHPSCKVTAMK |
| | | | CFLLELQVISLESGDASIHDTVENLIILANNSLSSNGNV |
| | | | 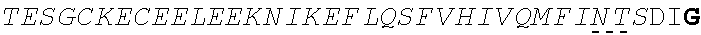 |
| | | | 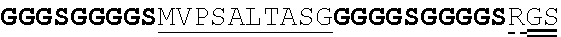 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| SB1902-C9- | hIgG Fc(hole)-lk3- | 41 | DKTHTCPPCPAPELLGGPSVFLFPPKPKDTLMISRT |
| variant2 | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 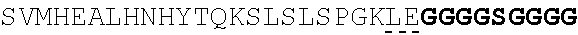 |
| | | | 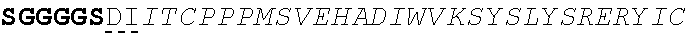 |
| | | | NSGFKRKAGTSSLTECVLNKATNVAHWTTPSLKCIR |
| | hIgG Fc(knob)-lk1- | 65 | DKTHTCPPCPAPELLGGPSVFLFPPKPKDTLMISRT |
| | IL15-lk2-CM1-lk2- | | PEVTCVVVDVSHEDPEVKFNWYVDGVEVHNAKT |
| | MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 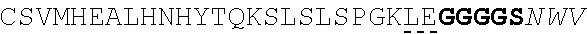 |
| | | | NVISDLKKIEDLIQSMHIDATLYTESDVHPSCKVTAMK |
| | | | CFLLELQVISLESGDASIHDTVENLIILANNSLSSNGNV |
| | | | 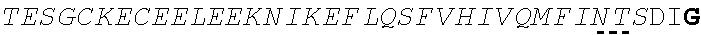 |
| | | | 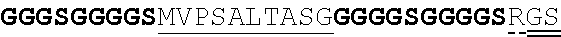 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| SB1902-C9- | hIgG Fc(hole)-lk3- | 41 | DKTHTCPPCPAPELLGGPSVFLFPPKPKDTLMISRT |
| variant2_L45D | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 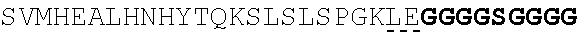 |
| | | | 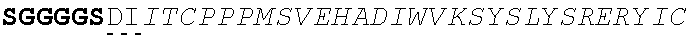 |
| | | | NSGFKRKAGTSSLTECVLNKATNVAHWTTPSLKCIR |
| | hIgG Fc(knob)-lk1- | 87 | DKTHTCPPCPAPELLGGPSVFLFPPKPKDTLMISRT |
| | IL15_L45D-lk2- | | PEVTCVVVDVSHEDPEVKFNWYVDGVEVHNAKT |
| | CM1-lk2-MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 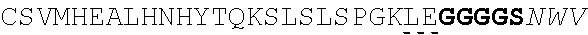 |
| | | | NVISDLKKIEDLIQSMHIDATLYTESDVHPSCKVTAMK |
| | | | CFLDELQVISLESGDASIHDTVENLIILANNSLSSNGNV |
| | | | 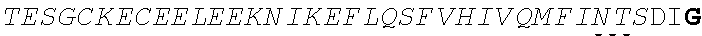 |
| | | | 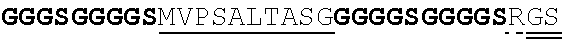 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| SB1902-C9- | hIgG Fc(hole)-lk3- | 41 | DKTHTCPPCPAPELLGGPSVFLFPPKPKDTLMISRT |
| variant2_L45E | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 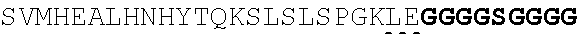 |
| | | | 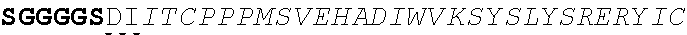 |
| | | | NSGFKRKAGTSSLTECVLNKATNVAHWTTPSLKCIR |
| | hIgG Fc(knob)-lk1- | 88 | DKTHTCPPCPAPELLGGPSVFLFPPKPKDTLMISRT |
| | IL15_L45E-lk2- | | PEVTCVVVDVSHEDPEVKFNWYVDGVEVHNAKT |
| | CM1-lk2-MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 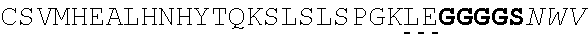 |
| | | | NVISDLKKIEDLIQSMHIDATLYTESDVHPSCKVTAMK |
| | | | CFLEELQVISLESGDASIHDTVENLIILANNSLSSNGNV |
| | | | 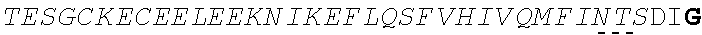 |
| | | | 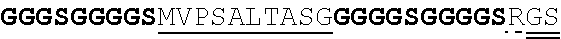 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| SB1902-C9- | hIgG Fc(hole)-lk3- | 41 | DKTHTCPPCPAPELLGGPSVFLFPPKPKDTLMISRT |
| variant2_Q48K | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 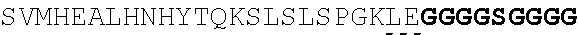 |
| | | | 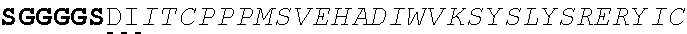 |
| | | | NSGFKRKAGTSSLTECVLNKATNVAHWTTPSLKCIR |
| | hIgG Fc(knob)-lk1- | 89 | DKTHTCPPCPAPELLGGPSVFLFPPKPKDTLMISRT |
| | IL15_Q48K-lk2- | | PEVTCVVVDVSHEDPEVKFNWYVDGVEVHNAKT |
| | CM1-lk2-MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 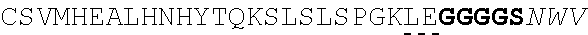 |
| | | | NVISDLKKIEDLIQSMHIDATLYTESDVHPSCKVTAMK |
| | | | CFLLELKVISLESGDASIHDTVENLIILANNSLSSNGNV |
| | | | 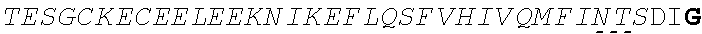 |
| | | | 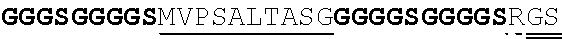 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| SB1902-C9- | hIgG Fc(hole)-lk3- | 41 | DKTHTCPPCPAPELLGGPSVFLFPPKPKDTLMISRT |
| variant2_S51D | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 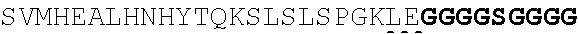 |
| | | | 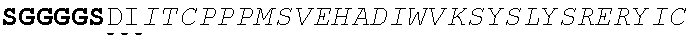 |
| | | | NSGFKRKAGTSSLTECVLNKATNVAHWTTPSLKCIR |
| | hIgG Fc(knob)-lk1- | 90 | DKTHTCPPCPAPELLGGPSVFLFPPKPKDTLMISRT |
| | IL15_S51D-lk2- | | PEVTCVVVDVSHEDPEVKFNWYVDGVEVHNAKT |
| | CM1-lk2-MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 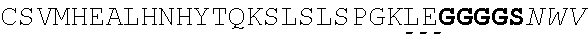 |
| | | | NVISDLKKIEDLIQSMHIDATLYTESDVHPSCKVTAMK |
| | | | CFLLELQVIDLESGDASIHDTVENLIILANNSLSSNGNV |
| | | | 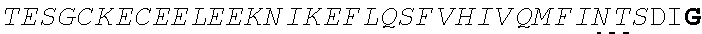 |
| | | | 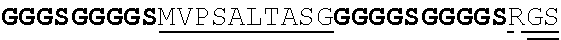 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| SB1902-C9- | hIgG Fc(hole)-lk3- | 41 | DKTHTCPPCPAPELLGGPSVFLFPPKPKDTLMISRT |
| variant2_L52D | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 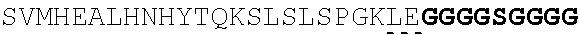 |
| | | | 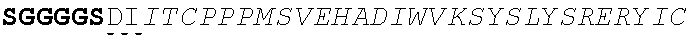 |
| | | | NSGFKRKAGTSSLTECVLNKATNVAHWTTPSLKCIR |
| | hIgG Fc(knob)-lk1- | 91 | DKTHTCPPCPAPELLGGPSVFLFPPKPKDTLMISRT |
| | IL15_L52D-lk2- | | PEVTCVVVDVSHEDPEVKFNWYVDGVEVHNAKT |
| | CM1-lk2-MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 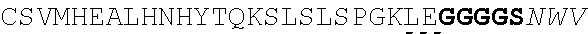 |
| | | | NVISDLKKIEDLIQSMHIDATLYTESDVHPSCKVTAMK |
| | | | CFLLELQVISDESGDASIHDTVENLIILANNSLSSNGNV |
| | | | 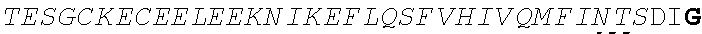 |
| | | | 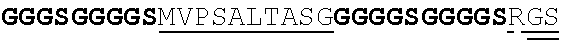 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| SB1902-C9- | hIgG Fc(hole)-lk3- | 41 | DKTHTCPPCPAPELLGGPSVFLFPPKPKDTLMISRT |
| variant2_E64K | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 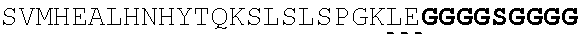 |
| | | | 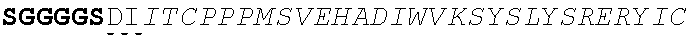 |
| | | | NSGFKRKAGTSSLTECVLNKATNVAHWTTPSLKCIR |
| | hIgG Fc(knob)-lk1- | 92 | DKTHTCPPCPAPELLGGPSVFLFPPKPKDTLMISRT |
| | IL15_E64K-lk2- | | PEVTCVVVDVSHEDPEVKFNWYVDGVEVHNAKT |
| | CM1-lk2-MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 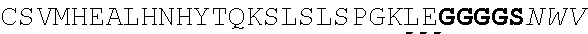 |
| | | | NVISDLKKIEDLIQSMHIDATLYTESDVHPSCKVTAMK |
| | | | CFLLELQVISLESGDASIHDTVKNLIILANNSLSSNGNV |
| | | | 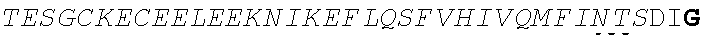 |
| | | | 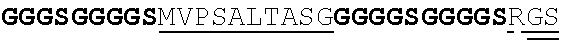 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| SB1902-C9- | hIgG Fc(hole)-lk3- | 41 | DKTHTCPPCPAPELLGGPSVFLFPPKPKDTLMISRT |
| variant2_I67D | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 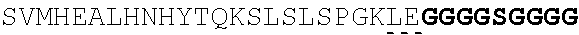 |
| | | | 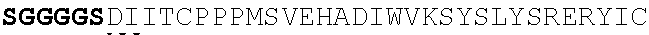 |
| | | | NSGFKRKAGTSSLTECVLNKATNVAHWTTPSLKCIR |
| | hIgG Fc(knob)-lk1- | 93 | DKTHTCPPCPAPELLGGPSVFLFPPKPKDTLMISRT |
| | IL15_I67D-lk2- | | PEVTCVVVDVSHEDPEVKFNWYVDGVEVHNAKT |
| | CM1-lk2-MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 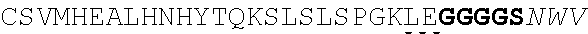 |
| | | | NVISDLKKIEDLIQSMHIDATLYTESDVHPSCKVTAMK |
| | | | CFLLELQVISLESGDASIHDTVENLDILANNSLSSNGN |
| | | | 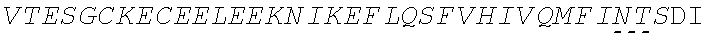 |
| | | | 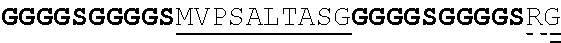 |
| | | | SPSGESSAPGSSSAESPGPGSESPSGSAPGESSPSGS |
| | | | APGSPSGESSAPGSPESGSASPGSSSAESPGPGSPSG |
| | | | ESSAPGSSPSGESSAPGSSSAESPGPGSESPSGSAPG |
| | | | ESSPSGSAPGSPSGESSAPGSPESGSASPGSSSAESP |
| | | | GPGSPSGESSAPGS |
| SB1902-C9- | hIgG Fc(hole)-lk3- | 41 | DKTHTCPPCPAPELLGGPSVFLFPPKPKDTLMISRT |
| variant2_I67E | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 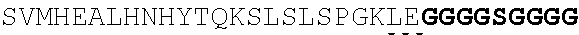 |
| | | | 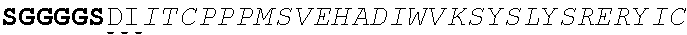 |
| | | | NSGFKRKAGTSSLTECVLNKATNVAHWTTPSLKCIR |
| | hIgG Fc(knob)-lk1- | 94 | DKTHTCPPCPAPELLGGPSVFLFPPKPKDTLMISRT |
| | IL15_167E-lk2- | | PEVTCVVVDVSHEDPEVKFNWYVDGVEVHNAKT |
| | CM1-lk2-MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 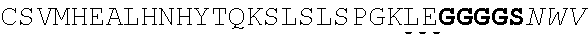 |
| | | | NVISDLKKIEDLIQSMHIDATLYTESDVHPSCKVTAMK |
| | | | CFLLELQVISLESGDASIHDTVENLEILANNSLSSNGN |
| | | | 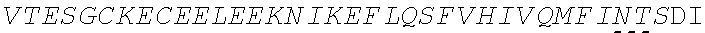 |
| | | | 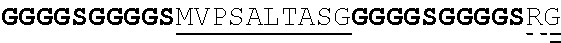 |
| | | | SPSGESSAPGSSSAESPGPGSESPSGSAPGESSPSGS |
| | | | APGSPSGESSAPGSPESGSASPGSSSAESPGPGSPSG |
| | | | ESSAPGSSPSGESSAPGSSSAESPGPGSESPSGSAPG |
| | | | ESSPSGSAPGSPSGESSAPGSPESGSASPGSSSAESP |
| | | | GPGSPSGESSAPGS |
| SB1902-C9- | hIgG Fc(hole)-lk3- | 41 | DKTHTCPPCPAPELLGGPSVFLFPPKPKDTLMISRT |
| variant2_I68D | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 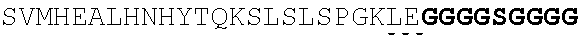 |
| | | | 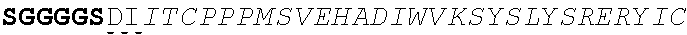 |
| | | | NSGFKRKAGTSSLTECVLNKATNVAHWTTPSLKCIR |
| | hIgG Fc(knob)-lk1- | 95 | DKTHTCPPCPAPELLGGPSVFLFPPKPKDTLMISRT |
| | IL15_I68D-lk2- | | PEVTCVVVDVSHEDPEVKFNWYVDGVEVHNAKT |
| | CM1-lk2-MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 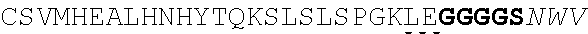 |
| | | | NVISDLKKIEDLIQSMHIDATLYTESDVHPSCKVTAMK |
| | | | CFLLELQVISLESGDASIHDTVENLIDLANNSLSSNGN |
| | | | 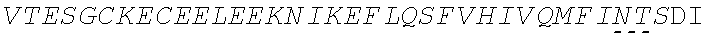 |
| | | | 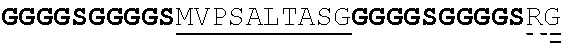 |
| | | | SPSGESSAPGSSSAESPGPGSESPSGSAPGESSPSGS |
| | | | APGSPSGESSAPGSPESGSASPGSSSAESPGPGSPSG |
| | | | ESSAPGSSPSGESSAPGSSSAESPGPGSESPSGSAPG |
| | | | ESSPSGSAPGSPSGESSAPGSPESGSASPGSSSAESP |
| | | | GPGSPSGESSAPGS |
| SB1902-C9- | hIgG Fc(hole)-lk3- | 41 | DKTHTCPPCPAPELLGGPSVFLFPPKPKDTLMISRT |
| variant2_N72D | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 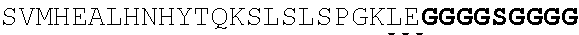 |
| | | | 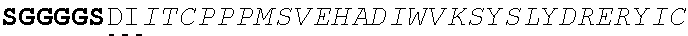 |
| | | | NSGFKRKAGTSSLTECVLNKATNVAHWTTPSLKCIR |
| | hIgG Fc(knob)-lk1- | 96 | DKTHTCPPCPAPELLGGPSVFLFPPKPKDTLMISRT |
| | IL15_N72D-lk2- | | PEVTCVVVDVSHEDPEVKFNWYVDGVEVHNAKT |
| | CM1-lk2-MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 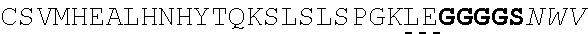 |
| | | | NVISDLKKIEDLIQSMHIDATLYTESDVHPSCKVTAMK |
| | | | CFLLELQVISLESGDASIHDTVENLIILANDSLSSNGNV |
| | | | 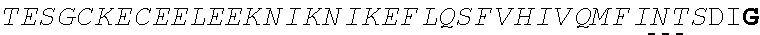 |
| | | | 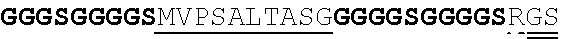 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| SB1902-C9- | hIgG Fc(hole)-lk1- | 66 | DKTHTCPPCPAPELLGGPSVFLFPPKPKDTLMISRT |
| variant3 | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 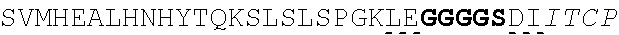 |
| | | | PPMSVEHADIWVKSYSLYSRERYICNSGFKRKAGTSSL |
| | | | TECVLNKATNVAHWTTPSLKCIR |
| | hIgG Fc(knob)-lk1- | 65 | DKTHTCPPCPAPELLGGPSVFLFPPKPKDTLMISRT |
| | IL15-lk2-CM1-lk2- | | PEVTCVVVDVSHEDPEVKFNWYVDGVEVHNAKT |
| | MP163 | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | 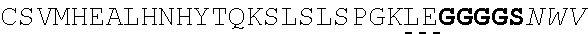 |
| | | | NVISDLKKIEDLIQSMHIDATLYTESDVHPSCKVTAMK |
| | | | CFLLELQVISLESGDASIHDTVENLIILANNSLSSNGNV |
| | | | 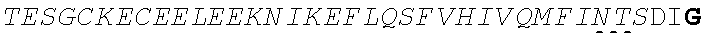 |
| | | | 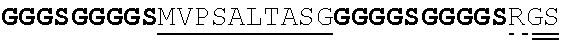 |
| | | | PSGESSAPGSSSAESPGPGSESPSGSAPGESSPSGSA |
| | | | PGSPSGESSAPGSPESGSASPGSSSAESPGPGSPSGE |
| | | | SSAPGSSPSGESSAPGSSSAESPGPGSESPSGSAPGE |
| | | | SSPSGSAPGSPSGESSAPGSPESGSASPGSSSAESPG |
| | | | PGSPSGESSAPGS |
| SB1902-C9- | hIgG Fc(hole- | 43 | DKTHTCPPCPAPEaaGGPSVFLFPPKPKDTLMISRTP |
| variant5 | LALA)-lk3- | | EVTCVVVDVSHEDPEVKFNWYVDGVEVHNAKTK |
| | IL15Rα_sushi | | PREEQYNSTYRVVSVLTVLHQDWLNGKEYKCKV |
| | | | SNKALPAPIEKTISKAKGQPREPQVYTLPPSREEMT |
| | | | KNQVSLSCAVKGFYPSDIAVEWESNGQPENNYKT |
| | | | TPPVLDSDGSFFLVSKLTVDKSRWQQGNVFSCSV |
| | | | 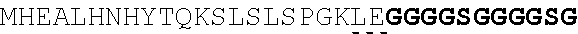 |
| | | | 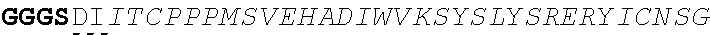 |
| | | | FKRKAGTSSLTECVLNKATNVAHWTTPSLKCIR |
| | hIgG Fc(knob- | 46 | DKTHTCPPCPAPEaaGGPSVFLFPPKPKDTLMISRTP |
| | LALA)-lk1-IL15- | | EVTCVVVDVSHEDPEVKFNWYVDGVEVHNAKTK |
| | lk2-CM1-lk2- | | PREEQYNSTYRVVSVLTVLHQDWLNGKEYKCKV |
| | MP163 | | SNKALPAPIEKTISKAKGQPREPQVYTLPPSREEMT |
| | | | KNQVSLWCLVKGFYPSDIAVEWESNGQPENNYK |
| | | | TTPPVLDSDGSFFLYSKLTVDKSRWQQGNVFSCS |
| | | | 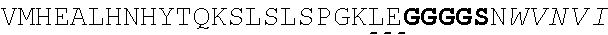 |
| | | | SDLKKIEDLIQSMHIDATLYTESDVHPSCKVTAMKCF |
| | | | LLELQVISLESGDASIHDTVENLIILANNSLSSNGNVTE |
| | | | 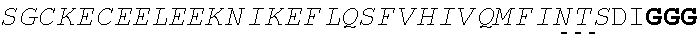 |
| | | | GSGGGGSMVPSALTASGGGGGSGGGGSRGSPSG |
| | | | ESSAPGSSSAESPGPGSESPSGSAPGESSPSGSAPGS |
| | | | PSGESSAPGSPESGSASPGSSSAESPGPGSPSGESSA |
| | | | PGSSPSGESSAPGSSSAESPGPGSESPSGSAPGESSP |
| | | | SGSAPGSPSGESSAPGSPESGSASPGSSSAESPGPGS |
| | | | PSGESSAPGS |
| SB1902-C9- | hIgG Fc(hole)-lk1- | 39 | DKTHTCPPCPAPELLGGPSVFLFPPKPKDTLMISRT |
| variant6 | IL15-lk2-CM1-lk2- | | PEVTCVVVDVSHEDPEVKFNWYVDGVEVHNAKT |
| | MP163 | | KPREEQYNSTYRVVSVLTVLHQDWLNGKEYKCK |
| | | | VSNKALPAPIEKTISKAKGQPREPQVYTLPPSREE |
| | | | MTKNQVSLSCAVKGFYPSDIAVEWESNGQPENNY |
| | | | KTTPPVLDSDGSFFLVSKLTVDKSRWQQGNVFSC |
| | | | 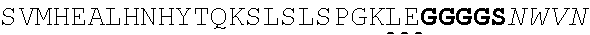 |
| | | | VISDLKKIEDLIQSMHIDATLYTESDVHPSCKVTAMKC |
| | | | FLLELQVISLESGDASIHDTVENLIILANNSLSSNGNVT |
| | | | 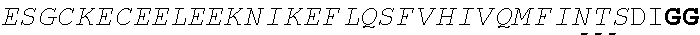 |
| | | | 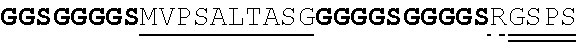 |
| | | | GESSAPGSSSAESPGPGSESPSGSAPGESSPSGSAPG |
| | | | SPSGESSAPGSPESGSASPGSSSAESPGPGSPSGESS |
| | | | APGSSPSGESSAPGSSSAESPGPGSESPSGSAPGESS |
| | | | PSGSAPGSPSGESSAPGSPESGSASPGSSSAESPGPG |
| | | | SPSGESSAPGS |
| | hIgG Fc(knob)-lk- | 40 | DKTHTCPPCPAPELLGGPSVFLFPPKPKDTLMISRT |
| | IL15Rα_sushi | | PEVTCVVVDVSHEDPEVKFNWYVDGVEVHNAKT |
| | | | KPREEQYNSTYRVVSVLTVLHQDWLNGKEYKCK |
| | | | VSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | CSVMHEALHNHYTQKSLSLSPGKGSGTCPPPMSV |
| | | | EHADIWVKSYSLYSRERYICNSGFKRKAGTSSLTECVL |
| | | | NKATNVAHWTTPSLKCIR |
| MBP-MP80 | MBP-MP80 | 48 | EEGKLVIWINGDKGYNGLAEVGKKFEKDTGIKVT |
| | | | VEHPDKLEEKFPQVAATGDGPDIIFWAHDRFGGY |
| | | | AQSGLLAEITPDKAFQDKLYPFTWDAVRYNGKLI |
| | | | AYPIAVEALSLIYNKDLLPNPPKTWEEIPALDKELK |
| | | | AKGKSALMFNLQEPYFTWPLIAADGGYAFKYEN |
| | | | GKYDIKDVGVDNAGAKAGLTFLVDLIKNKHMNA |
| | | | DTDYSIAEAAFNKGETAMTINGPWAWSNIDTSKV |
| | | | NYGVTVLPTFKGQPSKPFVGVLSAGINAASPNKEL |
| | | | AKEFLENYLLTDEGLEAVNKDKPLGAVALKSYEE |
| | | | ELAKDPRIAATMENAQKGEIMPNIPQMSAFWYAV |
| | | | RTAVINAASGRQTVDEALKDAQTSRGSPSGESSAP |
| | | | GSSSAESPGPGSESPSGSAPGESSPSGSAPGSPSGES |
| | | | SAPGSPESGSASPGSSSAESPGPGSPSGESSAP |

Production: the vectors coding the IL-15 prodrug, IL-15 drug or non-activatable IL-15 cytokine (i.e., SB1902-C4, without cleavable moiety) as shown in Table 2 were transiently transfected, and proteins were expressed in Expi293 cells (Thermo Fisher Scientific, Waltham, MA, USA) according to the manufacturer's protocol. The culture supernatant media were clarified by centrifugation and 0.2 μm membrane filtration. The exemplary IL-15 prodrugs, the exemplary IL-15 drugs and the non-activatable IL-15 cytokine SB1902-C4 as shown in Table 2 were purified by a two-step purification process comprising a precast MabSelect SuRe pcc column (Cytiva lifescience, Cat number 17549112) and size-exclusion chromatography (Superdex200, Cytiva, USA), according to the manufacturer's protocol. MBP-MP80 was a fusion protein of maltose binding protein and masking polypeptide MP80 (SEQ ID NO: 1), and was purified with a precast column with amylose resin (NEB, Cat number E8021L, USA) by following the manufacturer's instructions.

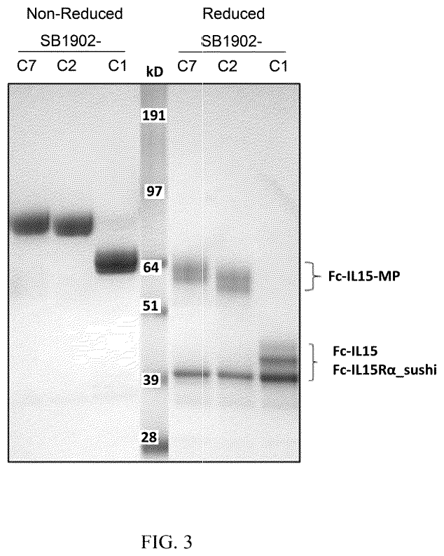

SDS-PAGE Analysis: The exemplary purified prodrugs SB1902-C2, SB1902-C7 and drug SB1902-C1 were analyzed with 4-20% polyacrylamide SDS-PAGE under reduced or non-reduced conditions for purity evaluation, the gel was stained using SimplyBlue SafeStain (FIG. 3).

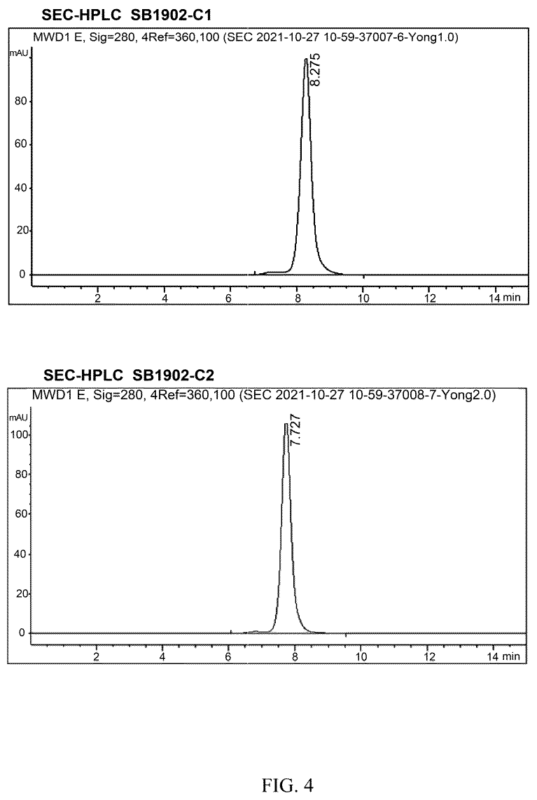

SEC-HPLC Analysis: the prodrug SB1902-C2 and drug SB1902-C1 were analyzed with analytic SEC-HPLC for homogeneity assessment (FIG. 4). HPLC analysis was performed using TSKgel G3000SWxl column according to the manufacturer's suggested running protocol. All purified drug and prodrugs were analyzed by both SDS-PAGE and SEC-HPLC in the same way.

The SDS-PAGE and HPLC results were shown in FIG. 3 and FIG. 4, this exemplary data demonstrate that constructs as described herein can be successfully produced, and the purified prodrugs SB1902-C2 and SB1902-C7 and the drug SB1902-C1 showed good purity and homogeneity.

### Example 3: Protease Cleavage of Prodrugs by MMP2 and MMP9

This experiment relates to the discovery of polypeptide substrates that are sensitive for MMP2 and MMP9, the efficiency of substrate cleavage can be measured by different methods.

In order to obtain a suitable polypeptide substrate that exhibits suitable sensitivity to both MMP2 and MMP9, a known substrate sequence (i.e., phage clone A3 with the sequence of AKPRALTA, from US patent Pub. NO.: US 2009/0253896 A1) was used as a template for mutagenesis. After mutagenesis and screening, a series of substrate sequences (cleavage moiety) for both MMP2 and MMP9 with suitable sensitivity were obtained, named CM1-CM10, of which the sequences and in vitro cleavage efficiency results were shown in Table 8.

As to the test of in vitro cleavage efficiency, an in vitro enzymatic analysis was carried out. Briefly, recombinant human MMP2 (rhMMP2) (R&D Systems, Cat number 902-MP-010) and recombinant human MMP9 (rhMMP9) (R&D Systems, Cat number 911-MP-010) were diluted to 100 μg/mL with Assay Buffer (50 mM Tris, 10 mM CaCl₂, 150 mM NaCl, 0.05% (w/v) Brij 35, pH 7.5), and the rhMMP2 and rhMMP9 were activated by 1 mM of APMA (4-aminophenylmercuric acetate, Sigma-Aldrich, USA). Briefly, one microgram of each of diluted MMP2 and MMP9 was aliquoted into Eppendorf tubes and p-aminophenylmercuric acetate (APMA) (Sigma, Catalog number A-9563) was added to a final concentration of 1 mM. The tubes were incubated at 37° C. for 2 hours for rhMMP2 and overnight for rhMMP9 to activate rhMMP-2 and rhMMP9. Activated rhMMP-2 or rhMMP9 was diluted to 1 ng/μL in Assay Buffer. Load 16 μL of activated rhMMP2 and rhMMP9 respectively into the first tube, add 4 μL of Assay buffer. Start to do 2 times serial dilution from tube 1 to tube 7 to get the final enzyme concentration from 400 ng/mL to 6.25 ng/mL. The protease digestion was carried out by adding 1.5 μg of SB1902-C2, SB1902-C5 or SB1902-C4 to each Eppendorf tube with preloaded active rhMM2 or rhMM9 with different enzyme concentrations for 5 hours at 37° C. After 5 hours of incubation, the prodrugs or drug prior to and after digestion were analyzed with 4-20% polyacrylamide gel.

TABLE 8

| | | | | | | | | | | | Cleavage | |
|---|---|---|---|---|---|---|---|---|---|---|---|---|
| | | | | | | | | | | | efficiency | |
| | Mutation site | | | | | | | | | | (EC50) (μg/ml) | |
| | P5 | P4 | P3 | P2 | P1 | P1′ | P2′ | P3′ | P4′ | P5′ | MMP2 | MMP9 |
| Template | A | K | P | R | A | L | T | A | | | | |
| CM1 | M | V | P | S | A | L | T | A | S | G | 0.006 | 0.05 |
| CM2 | M | V | L | S | A | V | T | A | S | G | 0.2 | >0.4 |
| CM4 | M | V | V | L | A | L | T | A | S | G | 0.15 | 0.2 |
| CM5 | M | V | L | S | A | P | T | A | S | G | >0.4 | ~ |
| CM6 | M | V | P | S | A | V | T | A | S | G | >0.4 | ~ |
| CM7 | M | V | P | S | A | Y | T | A | S | G | 0.1 | >0.4 |
| CM8 | M | V | A | S | A | L | T | A | S | G | 0.05 | >0.4 |
| CM9 | M | V | V | S | A | L | T | A | S | G | 0.025 | 0.1 |
| CM10 | M | V | P | S | A | L | T | V | S | G | 0.0125 | >0.4 |

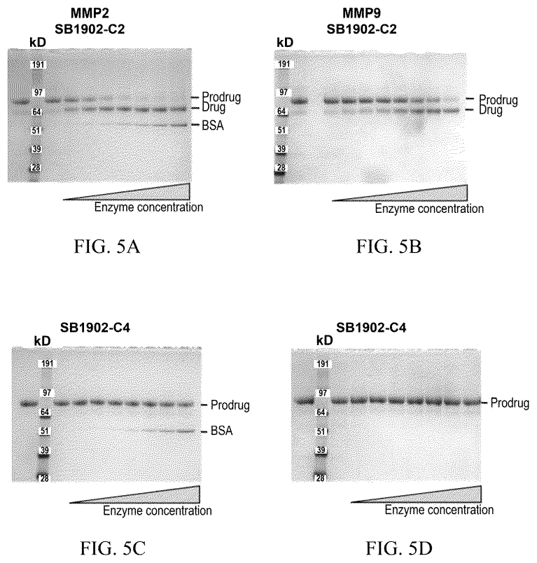

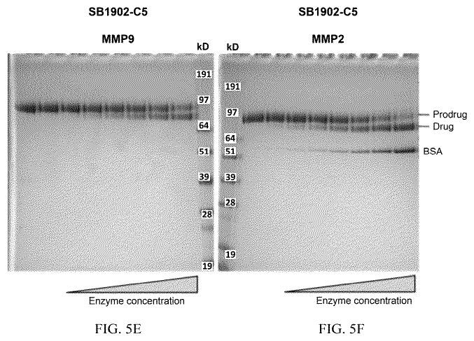

FIGS. 5A-5B depicted a proper cleavage profile of CM1 in prodrug SB1902-C2 after MMP2 and MMP9 digestion in vitro according to the protocol described above. The mutated substrate CM1 was cleavable in vitro by both MMP2 and MMP9 enzymes, with an enzyme dose-dependent manner.

FIGS. 5E-5F depicted a proper cleavage profile of CM4 in prodrug SB1902-C5 after MMP2 and MMP9 digestion in vitro according to the protocol described above. The mutated substrate CM4 was cleavable in vitro by both MMP2 and MMP9 enzymes, with an enzyme dose-dependent manner.

The other cleavable moieties (such as CM2, CM5-CM10) was also cleavable in vitro by both MMP2 and MMP9 (data not shown). These results indicated that the designed cleavable moieties in prodrugs could be cleaved at target tissue (e.g., tumor) in vivo.

As shown in FIGS. 5C-5D, however, the non-cleavable G4S linker in SB1902-C4 was resistant to both MMP2 and MMP9 enzyme digestion.

### Example 4: In Vitro Characterization of Prodrug and Drug

### 4.1 Nonspecific Serum Protein Binding:

the exemplary prodrug SB1902-C2 and SB1902-C3, and SB1902-C4 (without cleavable moiety) and drug SB1902-C1 binding to human, cynomolgus monkey, or rat serum proteins were assessed by direct ELISA. Briefly, human, cynomolgus monkey, or rat sera was coated on 96-well Maxisorp plates (Corning) at 25 μL per well at 4° C. overnight, respectively. The plates were then blocked in PBS with 1% BSA at room temperature for one hour. Serial dilution of SB1902-C2, SB1902-C3, SB1902-C4 or SB1902-C1 in PBS containing 0.5% BSA (ELISA buffer) was added to each plate for an hour and the plate was washed with PBS (wash buffer). Bound SB1902-C2, SB1902-C3, SB1902-C4 and SB1902-C1 were detected with anti-human IgG Fc specific antibody AP conjugated (Southern Biotech, Cat number 2014-04) in ELISA buffer. The plate was incubated at room temperature for one hour with agitation, washed six times with wash buffer, and developed for 3-10 minutes by addition of 50 μL/well of freshly prepared PNPP substrate (Sigma Aldrich, number N2770). Enzymatic color development was stopped with 50 μL/well of TMB stop solution (VWR, Cat number 95059-200). Plates were analyzed with a BioTek Gen5 plate reader (BioTek) at 405 nm.

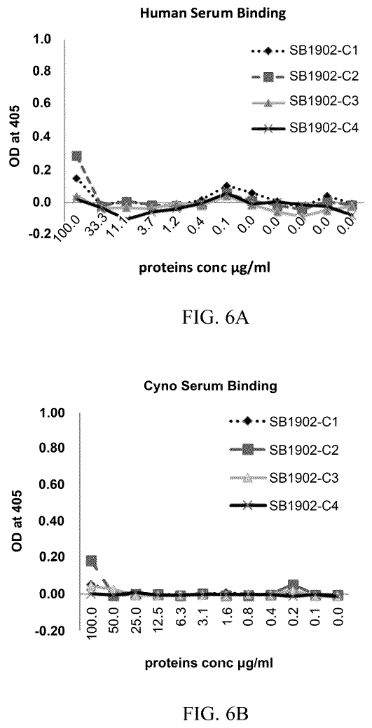

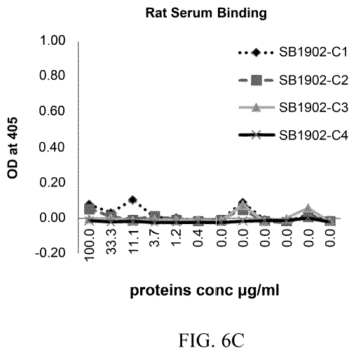

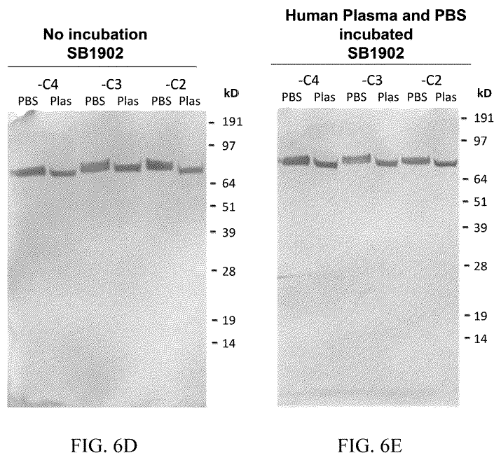

As shown in FIGS. 6A-6C, the prodrug SB1902-C2, SB1902-C3, SB1902-C4 and drug SB1902-C1 showed no detectable nonspecific binding with human, cynomolgus monkey, and rat serum protein.

### 4.2 Stability in Plasma:

The prodrugs in vitro serum stability was tested in human Plasma, cynomolgus plasma, and rat plasma. Blood obtained from human, cynomolgus, or rat was centrifuged to separate the blood cells from plasma. The top layer of plasma was carefully transferred to a new centrifuge tube and continued to centrifuge at 10000rcf for another 10 minutes. Five micrograms each of prodrug or drug was added into the tubes individually with 500 μL of human plasma, 500 μL of cynomolgus plasma or 500 μL of rat plasma, or 500 μL of PBS. The tubes were sealed and incubated at 40° C. for 7 days. Five microliter each of incubated prodrug and drug was separated by 4-20% precast polyacrylamide gel (Thermo Fisher, Cat number XP04205BOX). The separated proteins on the polyacrylamide gel were transferred to a PVDF membrane. After blocking and washing, the membranes were incubated with anti His6 polyclonal antibody with AP conjugates (SouthernBiotech, Cat number SB194b) for 1 hour followed by a wash. The blotted protein bands were developed with chromogenic reagent (Bio-Rad, Cat number 1706432) by following the manufacturer's instructions.

Representative western blot results were shown in FIGS. 6D-6E. The bands of prodrug SB1902-C2, SB1902-C3 and SB1902-C4 on WB membrane before/after incubation with plasma and PBS buffer were shown in FIG. 6D and FIG. 6E, which indicated that the prodrug SB1902-C2, SB1902-C3 and SB1902-C4 were incubated with the human plasma or PBS buffer without detectable degradation bands. Also, there were no detectable degradation bands shown on the WB membrane for both drug SB1902-C1 and prodrugs SB1902-C2, SB1902-C3, and SB1902-C4 after being incubated with cynomolgus monkey, or rat plasma, respectively (data was not shown). These results indicated that the prodrugs were stable in human, cynomolgus monkey and rat plasma.

### 4.3 Receptor Binding Affinity:

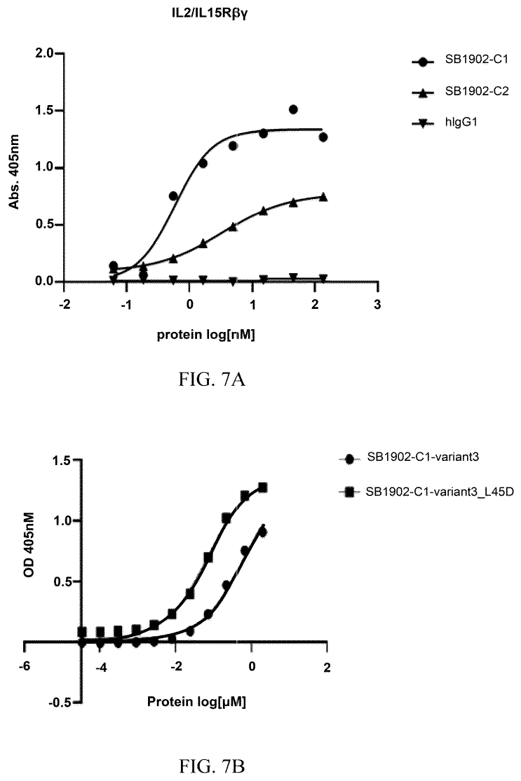

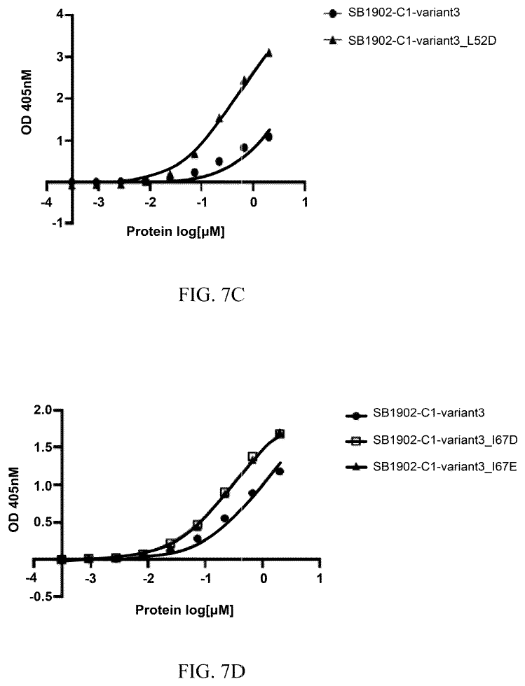

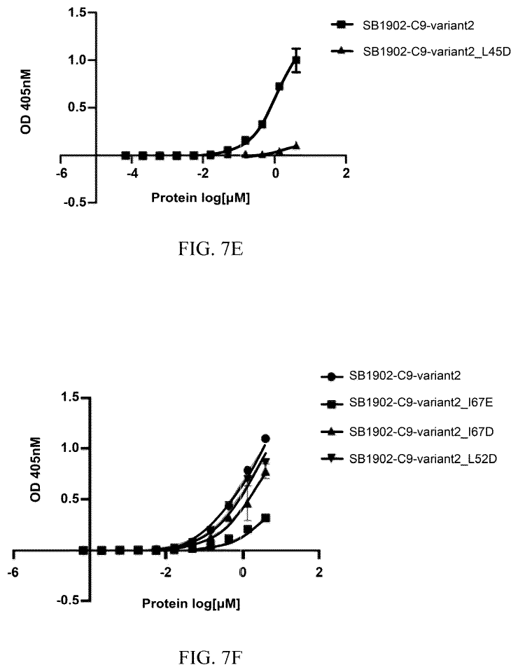

The binding affinity of the prodrug or drug to IL-2/IL-15Rβγ-Fc fusion protein was assessed by ELISA. The ELISA plate was coated with recombinant IL-15Rβγ-Fc at 1 μg/ml at 4° C. overnight. After blocking with 1% BSA in PBS and washing plate, a serial diluted exemplary prodrugs or drugs was loaded to the plate and incubated for 2 hours. Human IgG1 isotype antibody MOPC21, abbreviated as hIgG1 in FIG. 7A (see Hamlyn P H, Gait M J, Milstein C. (1981) Complete sequence of an immunoglobulin mRNA using specific priming and the dideoxynucleotide method of RNA sequencing. Nucleic Acids Res 9(18):4485-4494) was used as a negative control. After washing plate, an anti-human IgG-Fc-AP conjugated antibody at 1:2000 dilution was added to the individual well of the plate and incubated for 45 min. After washing plate, the AP substrate pNPP (Thermo, USA) was added and the optical densities were obtained at 405 nm with a spectrophotometer (BioTec, USA). The data were analyzed and plotted with GraphPad Prism8 software.

### 4.3.1 the Effect of Masking Polypeptide in the IL-15 Prodrugs on Receptor Binding Affinity of IL-15:

The exemplary prodrug SB1902-C2 and the drug SB1902-C1 were detected in this assay, as shown in FIG. 7A, the exemplary prodrug SB1902-C2 dramatically reduced the binding affinity to IL-2/IL-15Rβγ with comparison to the drug SB1902-C1. The result suggested that the masking polypeptide effectively hindered the receptor binding function of IL-15.

### 4.3.2 The Binding Affinity of the IL-15 Drugs or Prodrugs:

The exemplary drug SB1902-C1-variant3 with wild-type IL-15 and the drugs with IL-15 variants were detected in this assay. As shown in FIGS. 7B-7D, the exemplary drugs with IL-variants: SB1902-C1-variant3_L45D, SB1902-C1-variant3_L52D, SB1902-C1-variant3_I67D and SB1902-C1-variant3_I67E all showed higher binding affinity to IL-2/IL-15Rβ compared to the drug SB1902-C1-variant3.

The exemplary prodrug with wild-type IL-15 (SB1902-C9-variant2) and the prodrugs with IL-15 variants were also detected in this assay. The results, as shown in FIGS. 7E-7F, however, showed that the exemplary IL-15 prodrug SB1902-C9-variant2_L45D, SB1902-C9-variant2_L52D, SB1902-C9-variant2_I67D and SB1902-C9-variant2_I67E, exhibited reduced binding affinity to IL-2/IL-15Rβ compared to the prodrug SB1902-C9-variant2.

These results indicated that, in prodrugs with IL-15 variants, the masking polypeptide can also effectively hinder the binding function of IL-15 variant.

### Example 5: Immunogenicity Assessment

The immunogenicity of masking peptide (MP) was assessed by immunization of Balb/c mice with prodrug at 1 mg/kg per week through i.v. injection continuously for 6 weeks. Serum samples were collected from all mice on week 6 and analyzed for anti-prodrug antibodies by direct ELISA. The plate was coated with prodrug SB1902-C2 or MBP-MP80 overnight at 4° C., then blocked with 1% BSA in PBS. After washing plate, serially diluted sera from prodrug SB1902-C2 treated mice were added into the prodrug SB1902-C2 and MBP-MP80 pre-coated wells and serially diluted sera from Boco (Bococizumab, Pfizer, used as a positive control) treated mice were added to Boco pre-coated wells. After incubation and washing plate, goat anti-mouse IgG Fc-AP at 1:3000 dilution was added to the plates and incubated for 45 minutes. After washing plate, the freshly prepared substrate pNPP (Thermo, USA) was added to each well of the plate and the optical densities for each well were obtained using a spectrophotometer (BioTec, USA) set at 405 nm wavelength. The data were plotted using GraphPad Prism 8 software.

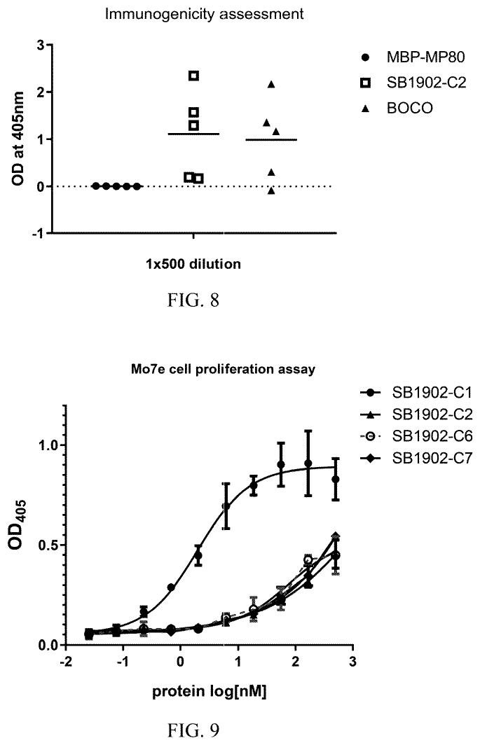

As shown in FIG. 8, all five mice with a weekly injection of prodrug for 6 weeks had not shown any detectable anti-drug antibody (ADA) from their sera against MBP-MP80 fusion protein by ELISA. In contrast, three out of five mice with a weekly injection of prodrug SB1902-C2 showed significant ADA from their sera against the Fc portion of the prodrug. The results indicated that the masking polypeptide MP80 in the prodrug is not immunogenic.

### Example 6: In Vitro Functional Experiments: Mo7e Cell Proliferation Assay

The prodrugs were also tested in vitro cell proliferation assay. It was known that IL-15 signal mainly through its binding with IL-2/IL-15Rβγ, Mo7e cells have both IL-2/IL15Rβ and γc expression on their cell membrane, and need IL-2 or IL-15 for their survival and proliferation.

To measure IL-15 cytokine-dependent Mo7e cell proliferation, the assay was performed essentially as follows: Mo7e cells (DSMZ, Cat number ACC 104) were maintained in RPMI-1640 medium supplemented with 10% FBS, 1% penicillin and streptomycin, and 10 ng/ml GM-CSF (Peprotech, Cat number 300-03) in the incubator at 37° C. (5% CO₂). Mo7e cells were harvested in their logarithmic growth phase and washed twice with GM-CSF free medium. Cells were plated in a 384-well plate with a cell number of 1×10⁴/well, and a medium volume of 45 μL/well (GM-CSF free). Cells were incubated for 2-4 hours in the incubator at 37° C. (5% CO₂). Prodrugs SB1902-C2, SB1902-C6, SB1902-C7 and drug SB1902-C1 were 3-times diluted with medium and 5 μL were added to the wells. Each concentration was repeated in triplicate. Cells with the only medium were set as a blank control. The cells were cultured in the incubator for 3 days. Then 5 μL of WST-8 (WST-8 Cell Proliferation Assay Kit, Cayman, Catalog number 10010199) was added to all wells and incubated in the incubator for 2-3 hours to test the proliferation of Mo7e cells. Absorbance at 450 nm (OD450) was detected. The data were analyzed and plotted with Graphpad Prism 8 software.

FIG. 9 showed that all the exemplary prodrugs SB1902-C2, SB1902-C6 and SB1902-C7 and drug SB1902-C1 had a dose-dependent stimulation of cell proliferation. However, the prodrugs SB1902-C2, SB1902-C6 and SB1902-C7 showed significantly reduced functionality compared to the drug SB1902-C1. The results demonstrated that the masking polypeptide MP80, MP96new and MP100 prevented IL-15 prodrug from binding to its receptor IL-2/IL-15Rβγ and reduced its signal transduction and downstream function.

### Example 7: In Vitro Functional Experiments: CD8+ T Cell Activation Assay

It was well documented that IL-15 promotes CD8+ memory T, natural killer (NK) and NKT cells proliferation, survival, and homeostasis. IL-15 causes T cell activation indicated by upregulation of the membrane surface expression of CD69 and cytokine release including IFN-γ. CD69 was an early activation marker of T cells. The percentage of CD8+ T cell activation was reflected by the percentage of CD69 surface expression.

### 7.1 the CD8+ T Cell-Based Activity of the IL-15 Drug or Prodrug

To compare the activity of the IL-15 prodrugs and IL-15 drugs in activating CD8+ T cell, this assay was performed essentially as follows: human PBMC (Stem Cell Technologies, Catalog number 70500) were plated with a cell number of 2×10⁵/well in 96 well round-bottom cell culture plate in RPMI-1640 medium supplemented with 10% FBS, 1% penicillin, and streptomycin. The exemplary IL-15 drugs and prodrugs were 3-times titrated with medium and 5 μL were added to the wells. Each concentration was repeated in triplicate, and blank wells (added with only medium) were used as a blank control. Cell plates were cultured in the incubator for 3 days. Then the cells were centrifuged and the cell pellet was stained with anti-CD3 Ab (BioLegend, cat #317306), anti-CD8 Ab (BioLegend, cat #344722), and anti-CD69 Ab (BioLegend, cat #310906) in FACS buffer for 30 minutes. They were washed twice with FACS buffer and acquired by flow cytometry with Attune (ThermoFisher Scientific). Data were analyzed with FlowJo software. The percentage of CD69+ cells in CD8+ T cells was plotted with Graphpad Prism 8 software.

### 7.1.1 the Effect of Masking Polypeptide in the IL-15 Prodrugs:

The exemplary prodrugs SB1902-C2, SB1902-C6, SB1902-C7, SB1902-C2-variant1, SB1902-C9-variant0, SB1902-C9-variant1, SB1902-C9-variant2, SB1902-C9-variant3, SB1902-C9-variant4 and SB1902-C10-variant1 and the exemplary drugs SB1902-C1, SB1902-C1-variant1, SB1902-C1-variant2 and SB1902-C1-variant3 were detected in this assay.

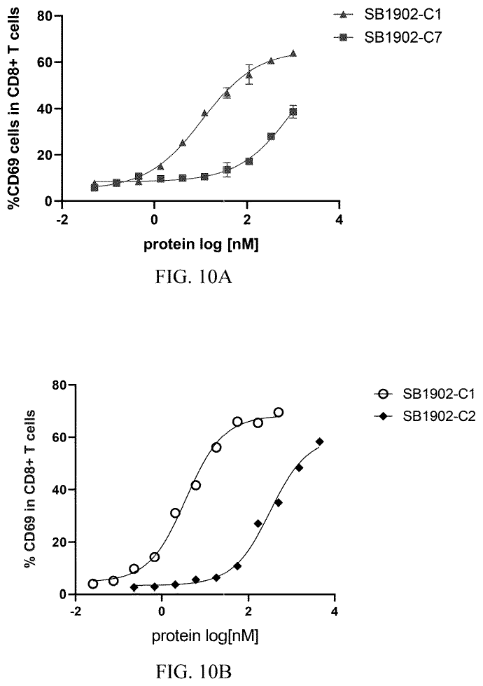

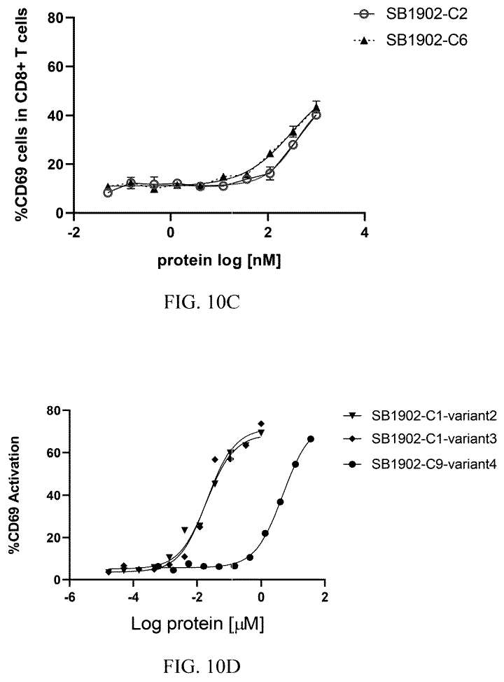

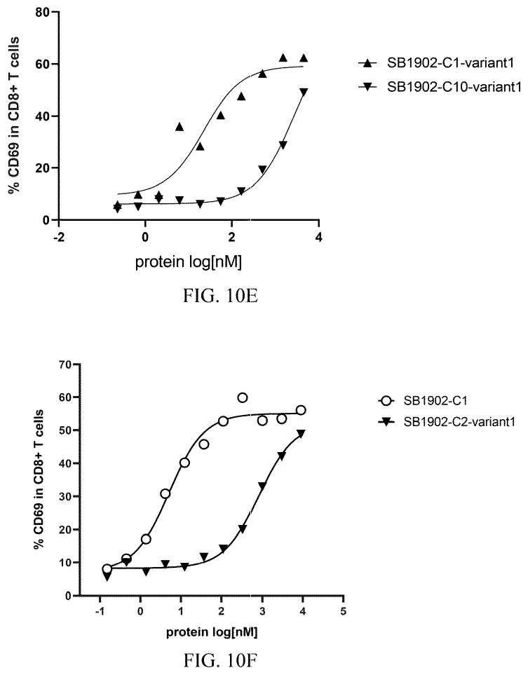

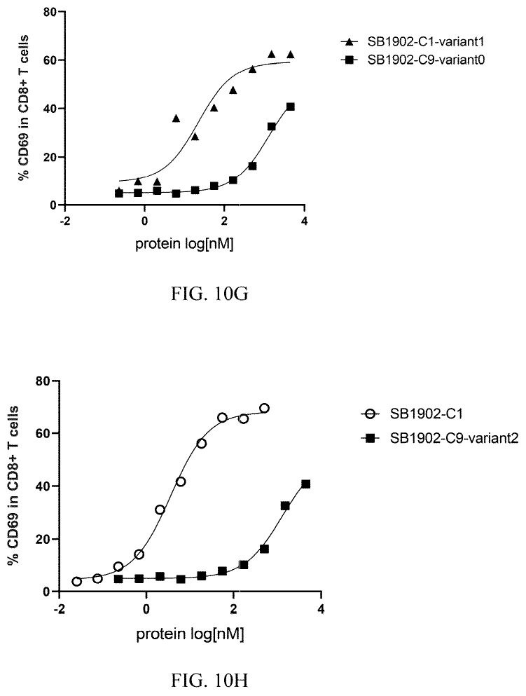

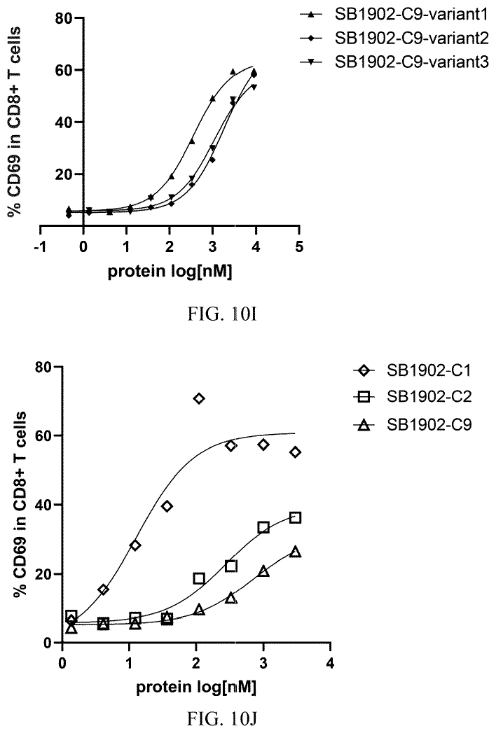

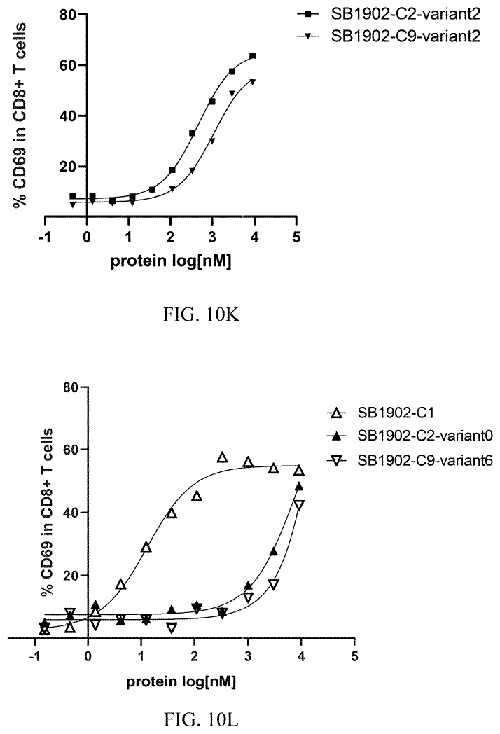

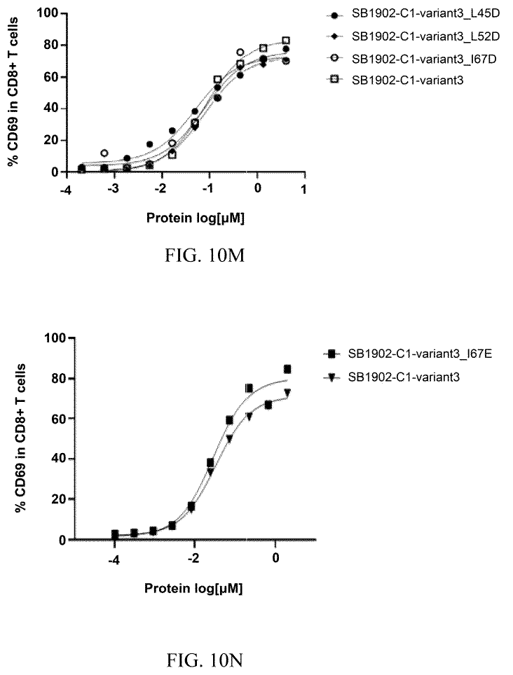

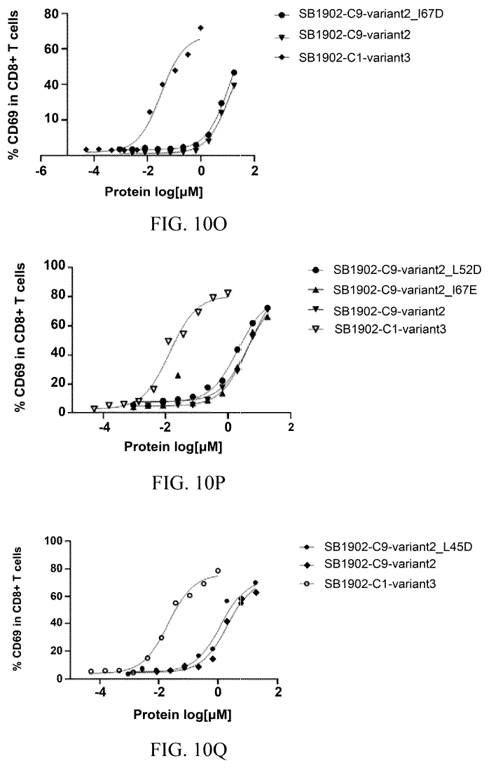

As shown in FIG. 10A, the prodrug SB1902-C7 with masking polypeptide MP100 had a significantly lower activity to stimulate T cell activation compared to the drug SB1902-C1.

As shown in FIG. 10B, the prodrug SB1902-C2 with masking polypeptide MP80 had a significantly lower activity to stimulate T cell activation compared to the drug SB1902-C1.

As shown in FIG. 10C, both the prodrug SB1902-C2 with masking polypeptide MP80 and the prodrug SB1902-C6 with masking polypeptide MP96new had low activity to stimulate T cell activation. Comparatively speaking, SB1902-C2 showed better efficacy in inhibiting CD69 activation in CD8+ T cells than SB1902-C6.

As shown in FIG. 10D, the prodrug SB1902-C9-variant4 with masking polypeptide MP163 and a LALA mutation (L234A and L235A) in Fc domain had a significantly lower activity to stimulate T cell activation compared to the drug SB1902-C1-variant2 and SB1902-C1-variant3.

As shown in FIG. 10E, the prodrug SB1902-C10-variant1 with masking polypeptide MP240 had a significantly lower activity to stimulate T cell activation compared to the drug SB1902-C1-variant1.

As shown in FIG. 10F, the prodrug SB1902-C2-variant1 with masking polypeptide MP80 had a significantly lower activity to stimulate T cell activation compared to the drug SB1902-C1.

As shown in FIG. 10G, the prodrug SB1902-C9-variant0 with masking polypeptide MP163 had a significantly lower activity to stimulate T cell activation compared to the drug SB1902-C1-variant1.

As shown in FIG. 10H, the prodrug SB1902-C9-variant2 with masking polypeptide MP163 had a significantly lower activity to stimulate T cell activation compared to the drug SB1902-C1.

As shown in FIG. 10I, the prodrugs SB1902-C9-variant1, SB1902-C9-variant2 and SB1902-C9-variant3 with masking polypeptide MP163 had low activity to stimulate T cell activation.

These results above demonstrated that the activity of IL15 cytokine in different prodrug formats with different masking polypeptides in activating T cells was significantly shielded by the masking polypeptides, demonstrated that the masking polypeptides of prodrugs prevented IL-15 from activating CD8+ T cells.

### 7.1.2 Different Length of the Masking Polypeptide (MP) in the IL-15 Prodrugs:

This assay was performed essentially as described above, the results of the prodrug SB1902-C9 and SB1902-C2 were shown in FIG. 10J. The results of the prodrug SB1902-C9-variant2 and SB1902-C2-variant2 were shown in FIG. 10K. And the results of the prodrug SB1902-C9-variant6 and SB1902-C2-variant0 were shown in FIG. 10L.

These results showed that the exemplary prodrugs with masking polypeptide all had a significantly lower activity to stimulate T cell activation compared to their corresponding drug SB1902-C1.

Moreover, comparatively speaking, in the FIGS. 10J-10L, the SB1902-C9 (FIG. 10J), SB1902-C9-variant2 (FIG. 10K) or SB1902-C9-variant6 (FIG. 10L) with masking polypeptide MP163 showed better efficacy in inhibiting CD69 activation in CD8+ T cells than the SB1902-C2 (FIG. 10J), SB1902-C2-variant2 (FIG. 10K) or SB1902-C2-variant0 (FIG. 10L) with masking polypeptide MP80 did, respectively, indicating that the masking polypeptide MP163 had better masking effect than MP80.

Combined with the results of FIG. 9 and FIGS. 10J-10L, the masking polypeptides with different lengths all had a good masking effect. The length of masking polypeptide in these tested lengths ranges from 80-100 amino acids, the small change in the MP length would not impact the activity much. However, the length of masking polypeptide plays some role when within the range of 80-163, there is some enhancements on the masking efficiency as the length increases.

7.1.3 the Activity of IL-15 Prodrugs or Drugs with IL-15 Variants:

This assay was performed essentially as described above, the exemplary drug with wild-type IL-15 (SB1902-C1-variant3) and the exemplary drugs with IL-15 variants (SB1902-C1-variant3_L45D, SB1902-C1-variant3_L52D, SB1902-C1-variant3_I67D and SB1902-C1-variant3_I67E) were detected in this assay.

The results, shown in FIG. 10M-10N, showed the IL-15 drug with the wild-type IL-15 and the drugs with IL-15 variants were all able to activate T cells, leading to an increased percentage of CD69 surface expression.

The exemplary prodrug with wild-type IL-15 (SB1902-C9-variant2) and the prodrugs with IL-15 variants (SB1902-C9-variant2_L45D, SB1902-C9-variant2_L52D, SB1902-C9-variant2_I67D and SB1902-C9-variant2_I67E) were also detected in this assay.

The results of these prodrugs, as shown in FIGS. 10O-10Q, showed that the prodrugs with IL-15 variants and the prodrug with wild-type IL-15 (SB1902-C9-variant2) all had significant decrease in the activity of activating T cells, when compared to the exemplary drug (SB1902-C1-variant3).

These results indicated that the masking polypeptide was able to exert a masking effect in either prodrug with wild-type IL-15 or prodrugs with IL-15 variants.

7.2 the Activity of Prodrug was Activated after Removing Masking Polypeptide by MMP2 Treatment

The masking polypeptide (MP) of the prodrug needs to be removed off to be a fully functional drug. To do that, the recombinant human MMP-2 (rhMMP2) (R&D Systems, Cat number 902-MP-010) was activated by following the manufacturer's instructions. Then, one microgram of activated MMP2 was added to an Eppendorf tube containing 250 μg of purified prodrug SB1902-C2, and the digestion volume was added to 0.5 mL with digestion buffer (50 mM Tris, 10 mM CaCl2, 150 mM NaCl, 0.05% (w/v) Brij 35, pH 7.5). The tube containing enzyme/prodrug mix was incubated at 37° C. for 5 hrs. After enzyme digestion, the sample was taken for evaluation of the digestion efficiency by SDS-PAGE. It was shown that about 90% of the prodrug SB1902-C2 was digested. The digested SB1902-C2 was purified with protein A beads to remove the enzyme and followed by buffer exchange and concentration. The final concentrate was applied to a human PBMC activation assay in which the comparison with the non-digested prodrug SB1902-C2 and the drug SB1902-C1 was done.

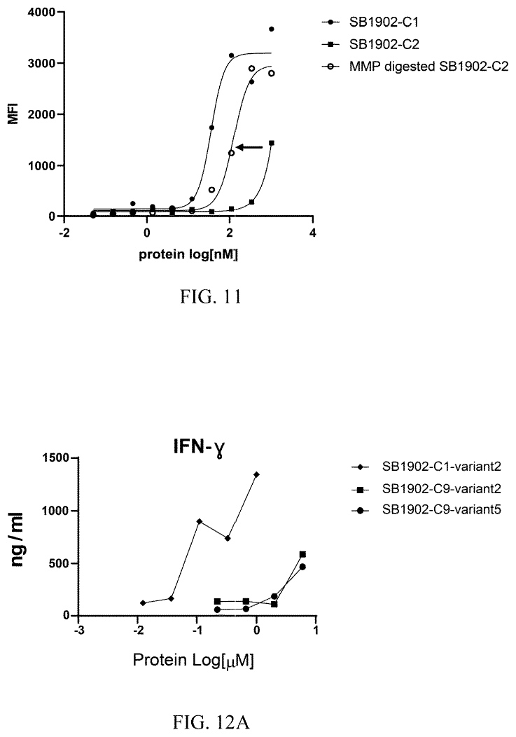

As shown in FIG. 11, after the masking polypeptide was removed from the exemplary prodrug SB1902-C2, the activity of MMP digested SB1902-C2 in the stimulation of CD8+ T cell activation was restored as compared to drug SB1902-C1.

### Example 8: In Vitro IFN-γ and Granzyme B Production Assay

T cell-derived IFN-γ is a key cytokine that stimulates innate immune responses. IL-15 has a significant role as an activator of T cell functions. It has been shown that IL-15 induces the expression of IFN-γ (Strengell M, et al. IL-21 in synergy with IL-15 or IL-18 enhances IFN-gamma production in human NK and T cells. J Immunol. 2003 Jun. 1; 170(11):5464-9). Overproduction of the proinflammatory cytokine IFN-γ systemically could cause unwanted side effects/toxicity in vivo.

Granzymes are serine proteases that are released by cytoplasmic granules within cytotoxic T cells and natural killer cells. Human Granzyme B is one of the five members of the human granzyme family which includes Granzymes A, B, G, H, and K. The IL-15 treatment resulted in increased NK and CD8+ T cell activation, the activation of NK and CD8+ T cell with features of enhanced IFN-γ production, proliferation (Ki67+), cytotoxic potential (Granzyme B production) and expression of the survival factor Bcl-2. (see Bergamaschi C, et al. Heterodimeric IL-15 delays tumor growth and promotes intratumoral CTL and dendritic cell accumulation by a cytokine network involving XCL1, IFN-γ, CXCL9 and CXCL10. J Immunother Cancer. 2020 May; 8(1):e000599).

The activity of the prodrugs on the IL-15 cytokine-dependent IFN-γ and Granzyme B production was detected essentially as follows: as mentioned above (e.g., in CD8+ T cell activation assay), Human PBMCs were stimulated with serially diluted IL-15 prodrugs or drugs. Cell plates were cultured in the incubator for 3 days. Then the cells were centrifuged at room temperature, 150×g for 5 min. The supernatant was collected for Granzyme B and IFN-7 detection. The human Granzyme B and IFN-γ concentrations in supernatant were quantified by ELISA using LEGEND MAX™ Human Granzyme B ELISA Kit (BioLegend, cat #439207) and Human IFN-γ ELISA MAX Deluxe kit (BioLegend, cat #430116), respectively. The data was analyzed and plotted with GraphPad Prism 8 software.

8.1 the Effect of Masking Polypeptide in the Prodrug with Wild-Type IL-15 on the IL-15 Cytokine-Dependent IFN-γ and Granzyme B Production

The exemplary prodrugs SB1902-C1-variant2, SB1902-C9-variant2 and SB1902-C9-variant5 were detected in this assay.

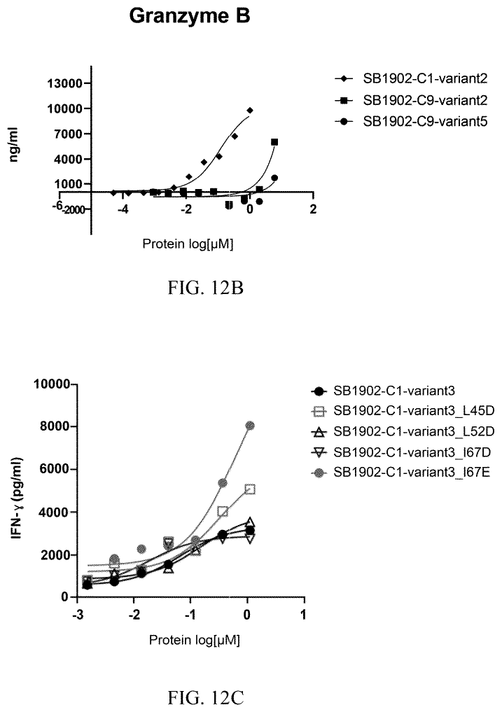

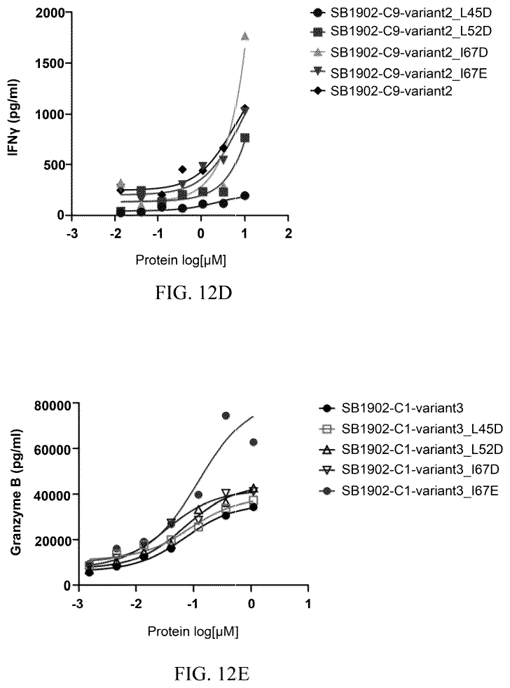

FIGS. 12A-12B showed that PBMCs stimulated by the exemplary prodrug SB1902-C9-variant2 or SB1902-C9-variant5 had less IFN-γ (FIG. 12A) and Granzyme B (FIG. 12B) production compared to that by the drug SB1902-C1-variant2 without masking polypeptide, demonstrating the masking polypeptide in the IL-15 prodrug can hinder the activity of IL-15 on the IL-15 cytokine-dependent IFN-γ and Granzyme B production.

8.2 the Activity of Prodrugs and Drugs with IL-15 Variants on the IFN-γ and Granzyme B Production

The exemplary IL-15 drug and prodrug with wild-type IL-15 (SB1902-C1-variant3, SB1902-C9-variant2) or with IL-15 variants were also detected in IFN-γ and Granzyme B production assays respectively.

### IFN-γ Production Assay:

The result of the drugs with wild-type IL-15 or IL-15 variants (FIG. 12C) showed that cells incubated with the exemplary drugs with IL-15 variants all had comparable or even higher efficacy in stimulation of T cell production of IFN-γ compared to the drug with wild-type IL-15 (SB1902-C1-variant3).

The result of the prodrugs with wild-type IL-15 or IL-15 variants (FIG. 12D) showed that cells incubated with the prodrugs with IL-15 variants exhibited even reduced production of IFN-γ, particularly with the variant L45D, by comparison to the parental prodrug with wild-type IL-15 (SB1902-C9-variant2) which as already shown previously had significantly reduced production of IFN-γ compared to the drug.

Combining with the FIG. 12C and FIG. 12D, it can be seen that the masking polypeptide in the prodrug with IL-15 variants can also reduce the activity of IL-15 variants on the IL-15 cytokine-dependent IFN-γ production, exerting its masking effect.

### Granzyme B Production Assay:

The result of the drugs with wild-type IL-15 or IL-15 variants (FIG. 12E) showed that cells incubated with the exemplary drugs with IL-15 variants had higher efficacy in stimulation of T cell production of Granzyme B compared to the drug with wild-type IL-15 (SB1902-C1-variant3).

The result of the prodrugs with wild-type IL-15 or IL-15 variants (FIG. 12F), however showed that cells incubated with the prodrugs with IL-15 variants exhibited reduced production of Granzyme B, particularly with the variant L45D, by comparison to the parental prodrug with wild-type IL-15 (SB1902-C9-variant2) which as already shown previously had significantly reduced production of Granzyme B compared to the drug.

Combining with the FIG. 12E and FIG. 12F, it can be seen that the masking polypeptide in the prodrug with IL-15 variants can also reduce the activity of IL-15 variants on the IL-15 cytokine-dependent Granzyme B production, exerting its masking effect.

In conclusion, these results showed that IL-15 prodrugs exhibited reduced stimulation of T cell production of Granzyme B and IFN-γ would potentially be translated to higher dosing and less systemic immune response when used in vivo compared to the drug, while the masking polypeptide gets cleaved off from the prodrug in the tumor tissue and exert therapeutic effects.

Moreover, the above results, which were consistent with the receptor binding affinity assay described in Example 4, also demonstrated that the IL-15 variants exhibited enhanced binding activity to the receptor IL-2/IL-15Rβ when compared to their parental wild-type IL-15, which were also positively correlated with their higher activities in stimulation of Granzyme B and IFN-γ production. However, these IL-15 variants in the prodrugs exhibited more reduced binding activity to the receptor compared to prodrug with wild-type IL-15, which were also correlated with a more decrease of Granzyme B and IFN-γ production. It could potentially be translated to a wider dosing window with less systemic toxicity in clinic practice when compared to the prodrug with wild-type IL-15.

### Example 9: In Vivo IFN-γ Production Assay

The IFN-γ production was also assayed in mouse, to measure if the masking polypeptide in the prodrug effect the IL-15 cytokine-dependent IFN-γ production in vivo, the assay was performed essentially as follows:

The drug SB1902-C1 or the prodrug SB-1902-C2 as an example was intravenously injected into Balb/c mice with the same molarity. Plasma was collected at 6 and 24 hours after injection. IFN-γ was detected with an ELISA kit (ThermoFisher Scientific, Cat #KMC4021C) according to the instruction.

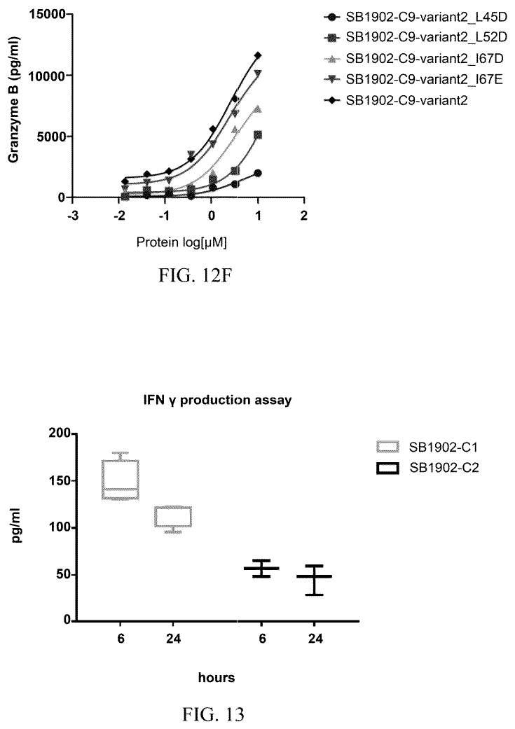

As shown in FIG. 13, mice treated with the exemplary prodrug SB1902-C2 had less IFN-γ production compared to the drug SB1902-C1 without masking polypeptide. The data indicated that the reduction of IFN-γ production induced by IL-15 in mice treated with prodrug is resulted from the prevention of IL-15 function by the masking polypeptide in prodrug. This would potentially translate to a lower systemic immune response by SB1902-C2 when it is used in vivo.

### Example 10: In Vivo Tumor Models to Evaluate Activity of Prodrug and Drug

The ability of the IL-15 prodrug and drug without masking polypeptide to promote tumor eradication and inhibit metastasis is assessed in vivo using the mouse WEHI-164 tumor model. A human IgG1 isotype antibody MOPC21 (see Hamlyn P H, Gait M J, Milstein C. (1981) Complete sequence of an immunoglobulin mRNA using specific priming and the dideoxynucleotide method of RNA sequencing. Nucleic Acids Res 9(18):4485-4494) was used as a control in this experiment.

### A. In Vivo Activity of Prodrug in WEHI-164 Subcutaneous Tumor Model

Animals and husbandry: Female mice (7-9 weeks of age) were used in the studies. The animals were fed irradiated Harlan 2918.15 Rodent Diet and water ad libitum. Animals were ear-tagged for identification purposes and shaved on the left dorsal flank area in preparation for cell implantation. Animals were housed in Innovive disposable ventilated caging with corn cob bedding at 60 complete air changes per hour. The environment was controlled to a temperature range of 70°±2° F. and a humidity range of 30-70%. All procedures carried out in this experiment were conducted by skilled personnel in compliance with all the laws, regulations, and guidelines of the National Institutes of Health (NIH) and with the approval of Biomere's Animal Care and Use Committee (Richmond, CA).

Cell preparation and implantation: WEHI-164 cells were obtained from ATCC (CAT #: CRL-1751™). WEHI-164 cells were cultured and expanded in Dulbecco's Modified Eagles Medium (DMEM) with 2 mM L-glutamine, 10% fetal bovine serum (FBS), and 1% 100× Penicillin/Streptomycin (PS). The growth environment was maintained in an incubator with a 5% CO₂ atmosphere at 37° C. When the expansion was complete, the cells were trypsinized using a 0.25% trypsin-EDTA solution. The cells were then washed and counted. Pre-implantation cell viability was >95%. The cells were resuspended in Dulbecco's Phosphate Buffered Saline (DPBS). Test animals were sterilized at the implantation site with an alcohol prep pad and were implanted subcutaneously on Day 0 in 0.1 mL using a 25-gauge needle and 1 mL syringe.

Measurements and treatment: Tumors were allowed to grow at the range of 70-150 mm³ and were then randomized into study groups. Mice were distributed to ensure that the mean body weights for all groups were within 10% of the overall mean tumor burden for the study population. Mice were intravenously injected twice weekly with a dose of 3 mg/kg of human IgG1 isotype antibody or the prodrug SB1902-C2 for 2 weeks and tumor volumes were monitored.

Assessment of side effects: All animals were observed for clinical signs of distress or toxicity at least once daily. Animals were weighed once per week. If an individual animal showed overt signs of distress or 15% body weight loss, the individual animal was weighed daily. Animals were euthanized if bodyweight loss was in excess of 20% or other clinical signs warranted euthanasia. Individual animals were euthanized when their tumor volume reached or exceeded 2000 mm³.

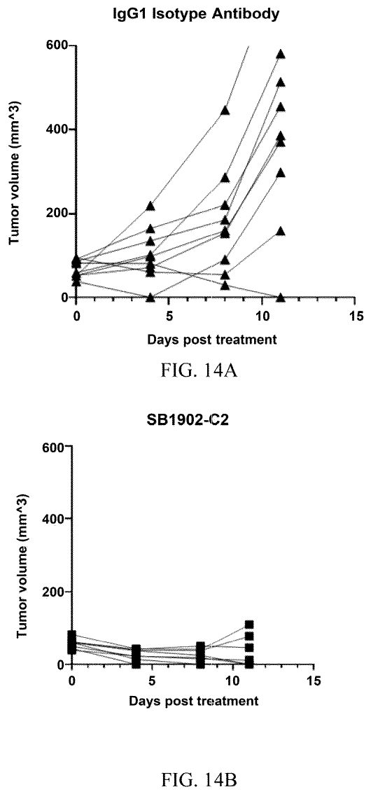

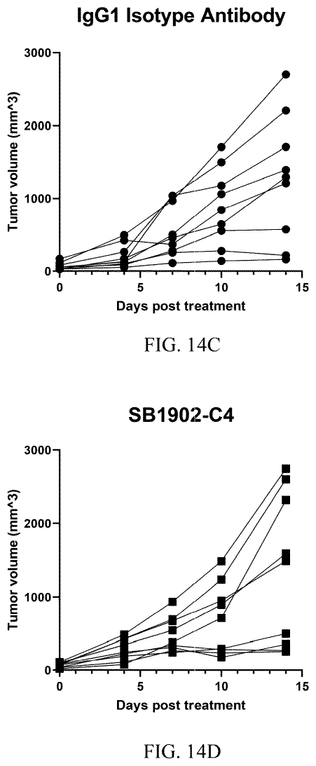

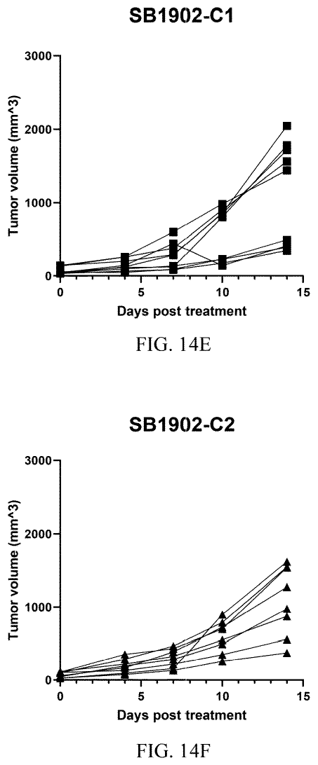

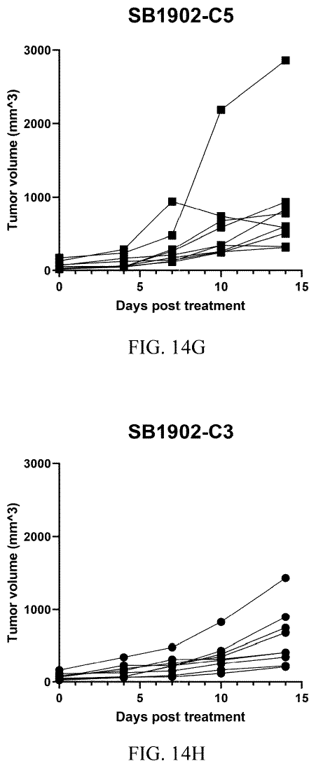

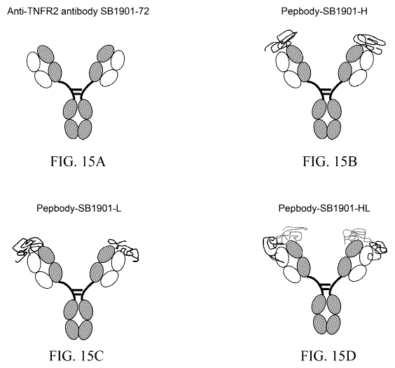

Results: As shown in FIGS. 14A-14B, animals with WEHI-164 tumor were treated with human IgG1 isotype control antibody or exemplary prodrug SB1902-C2. All animals in the study did not show overt signs of systemic toxicity throughout the treatment course. The tumor growth in SB19020-C2 treated animals (FIG. 14B) was dramatically inhibited compared to the tumor growth in animals treated with isotype control antibody (FIG. 14A). This result demonstrated that the prodrug can function in the tumor bearing mice, which suggested that the masking polypeptide can be cleaved in situ and IL-15 was released and functioned in the tumor microenvironment.

B. In Vivo Activity of Prodrug with Different Cleavable Moiety (CM) in WEHI-164 Subcutaneous Tumor Model:

To test the activity of different prodrug constructs with different cleavable moiety in the WEHI-164 tumor mouse model, the procedure was the same as described above. the exemplary prodrug SB1902-C2, SB1902-C3, and SB1902-C5, the drug SB1902-C1, and SB1902-C4 without cleavable moiety (CM) were tested in this assay. A human IgG1 isotype antibody MOPC21 (see Hamlyn P H, Gait M J, Milstein C. (1981) Complete sequence of an immunoglobulin mRNA using specific priming and the dideoxynucleotide method of RNA sequencing. Nucleic Acids Res. 9(18):4485-4494) was used as a control in this experiment. Prodrugs were intravenously injected at a dose of 1 mg/kg for all groups except the drug SB1902-C1 group which was dosed at 0.3 mg/kg which was MTD based on previous dosing experiments. The dosing time was at day0, day4, day7, day10 and day14. Tumor volumes were monitored.

Results: As shown in FIGS. 14C-14H, tumor size in the isotype control antibody group (FIG. 14C) and SB1902-C4 group (FIG. 14D) did not show much difference, indicating that the non-cleavable linker in SB1902-C4 could not be cleaved by the enzymes in the tumor and the masking peptide was not removed from the complex, thus IL-15 in SB1902-C4 did not show any function. However, tumor growth in SB1902-C2 (FIG. 14F), SB1902-C3 (FIG. 14H), and SB1902-C5 (FIG. 14G) groups was remarkedly reduced and no significant difference was observed among these groups even there is one outlier in the group SB1902-C5 treatment. These results strongly suggested that the cleavable moiety in these groups were cleaved and IL-15 prodrug was activated in the tumor microenvironment, and the tumor cell was inhibited by the activated IL-15 prodrug. In addition, the tumor growth in the SB1902-C1 group (FIG. 14E) was only partially inhibited, suggesting that the maximal tolerable IL-15 dose (MTD) couldn't achieve optimal antitumor efficacy. Therefore, these results demonstrated that the masking polypeptide not only reduced the toxicity of IL-15, and also that the CM in the prodrug was cleavable on the predictable site in the tumor microenvironment.

### Example 11: Construction and Expression of Masked Anti-TNFR2 Antibody Prodrug

The effect of masking polypeptide has been validated in cytokine prodrugs as described above. The following assays identified the effect of masking polypeptides described in antibody prodrugs, e.g., to test if the masking polypeptides were capable of blocking the antibody from binding to the antigen.

The anti-TNFR2 antibody SB1901-72 provided herein was described in U.S. App. No. 63/219,796, which was incorporated by reference herein in its entirety.

For masked antibody constructs, the masking polypeptide was added to the N-terminal of the heavy chain of SB1901-72 via a cleavable moiety (CM), named Pepbody-SB1901-H; the masking polypeptide was added to the N-terminal of the light chain of SB1901-72 via a cleavable moiety (CM), named Pepbody-SB1901-L; the masking polypeptide was added to both the N-terminal of the heavy chain and N-terminal of the light chain of SB1901-72 via a cleavable moiety (CM), named Pepbody-SB1901-HL. The antibody prodrug constructs were recombinantly expressed in HEK 293 cells, and purified as described in Example 2. The CDR sequences of the SB1901-72 antibody were shown in Table 9, wherein the CDR numbering is according to the EU index of Kabat. The V_{H} and V_{L} sequences of the SB1901-72 antibody were shown in Table 10. The construct description of the antibody prodrug was shown in Table 11. The sequences of masked anti-TNFR2 antibody prodrug were shown in Table 12. The illustrative structure of the anti-TNFR2 antibody was shown in FIG. 15A, the illustrative structure of masked antibody prodrug: Pepbody-SB1901-H, Pepbody-SB1901-L and Pepbody-SB1901-HL were shown in FIGS. 15B-15D.

TABLE 9

| Antibody | | | |
|---|---|---|---|
| Name | HC-CDR1 | HC-CDR2 | HC-CDR3 |
| SB1901-72 | DDYID | EIYPGSGNTYYNEKFKG | SQVYGKIAMDH |
| | (SEQ ID NO: 50) | (SEQ ID NO: 51) | (SEQ ID NO: 52) |
| Antibody | | | |
| Name | LC-CDR1 | LC-CDR2 | LC-CDR3 |
| SB1901-72 | RASESVDNSGNSFMH | RASNLES | QQSKEDPYT |
| | (SEQ ID NO: 53) | (SEQ ID NO: 54) | (SEQ ID NO: 55) |

TABLE 10

| | SEQ ID NO: | Sequence |
|---|---|---|
| SB1901-72 V_{H} | SEQ ID NO: 56 | QVQLVQSGAEVKKPGASVKVSCKASGYTFTDDY |
| | | IDWVRQATGQGLEWIGETYPGSGNTYYNEKFKGR |
| | | VTMTRDKSISTAYMELSSLRSEDTAVYYCARSQV |
| | | YGKIAMDHWGQGTLVTVSS |
| SB1901-72 V_{L} | SEQ ID NO: 57 | DIVMTQSPDSLAVSLGERATINCRASESVDNSGNS |
| | | FMHWYQQKPGQPPKLLIYRASNLESGVPDRFSGS |
| | | GSRTDFTLTISSLQAEDVAVYYCQQSKEDPYTFG |
| | | QGTKVEIK |

TABLE 11

| | Construct description (SEQ ID NO:) | |
|---|---|---|
| Construct name | Heavy chain | Light chain |
| Pepbody-SB1901-H | MP80-lk2-CM1-lk2-V_{H}-C_{H}1-hinge- | V_{L}-C_{L} |
| | C_{H}2-C_{H}3 | (SEQ ID NO: 59) |
| | (SEQ ID NO: 58) | |
| Pepbody-SB1901-L | V_{H}-C_{H}1-hinge-C_{H}2-C_{H}3 | MP80-lk2-CM1-lk2-V_{L}-C_{L} |
| | (SEQ ID NO: 60) | (SEQ ID NO: 61) |
| Pepbody-SB1901-HL | MP80-lk2-CM1-lk2-V_{H}-C_{H}1-hinge- | MP80-lk2-CM1-lk2-V_{L}-CL |
| | C_{H}2-C_{H}3 | (SEQ ID NO: 61) |
| | (SEQ ID NO: 58) | |

TABLE 12

| Construct name | Sequences |
|---|---|
| Pepbody-SB1901-H | GSPSGESSAPGSSSAESPGPGSESPSGSAPGESSPSGSAPGSPSGESSAPGS |
| heavy chain | PESGSASPGSSSAESPGPGSPSGESSAPGGGGSGGGGSMVPSALTASGG |
| (SEQ ID NO: 58) | GGGSGGGGSQVQLVQSGAEVKKPGASVKVSCKASGYTFTDDYIDWVR |
| | QATGQGLEWIGEIYPGSGNTYYNEKFKGRVTMTRDKSISTAYMELSSL |
| | RSEDTAVYYCARSQVYGKIAMDHWGQGTLVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCPP |
| | CPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGF |
| | YPSDIAVEWESNGQPENNYKTTPPVLDSDGSFFLYSKLTVDKSRWQQG |
| | NVFSCSVMHEALHNHYTQKSLSLSPGK |
| Pepbody-SB1901-H | DIVMTQSPDSLAVSLGERATINCRASESVDNSGNSFMHWYQQKPGQPP |
| light chain | KLLIYRASNLESGVPDRFSGSGSRTDFTLTISSLQAEDVAVYYCQQSKE |
| (SEQ ID NO: 59) | DPYTFGQGTKVEIKRTVAAPSVFIFPPSDEQLKSGTASVVCLLNNFYPRE |
| | AKVQWKVDNALQSGNSQESVTEQDSKDSTYSLSSTLTLSKADYEKHK |
| | VYACEVTHQGLSSPVTKSFNRGEC |
| Pepbody-SB1901-L | QVQLVQSGAEVKKPGASVKVSCKASGYTFTDDYIDWVRQATGQGLE |
| heavy chain | WIGEIYPGSGNTYYNEKFKGRVTMTRDKSISTAYMELSSLRSEDTAVY |
| (SEQ ID NO: 60) | YCARSQVYGKIAMDHWGQGTLVTVSSASTKGPSVFPLAPSSKSTSGGT |
| | AALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLG |
| | GPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAP |
| | IEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSV |
| | MHEALHNHYTQKSLSLSPGK |
| Pepbody-SB1901-L | GSPSGESSAPGSSSAESPGPGSESPSGSAPGESSPSGSAPGSPSGESSAPGS |
| light chain | PESGSASPGSSSAESPGPGSPSGESSAPGGGGSGGGGSMVPSALTASGG |
| (SEQ ID NO: 61) | GGGSGGGGSDIVMTQSPDSLAVSLGERATINCRASESVDNSGNSFMHW |
| | YQQKPGQPPKLLIYRASNLESGVPDRFSGSGSRTDFTLTISSLQAEDVAV |
| | YYCQQSKEDPYTFGQGTKVEIKRTVAAPSVFIFPPSDEQLKSGTASVVC |
| | LLNNFYPREAKVQWKVDNALQSGNSQESVTEQDSKDSTYSLSSTLTLS |
| | KADYEKHKVYACEVTHQGLSSPVTKSFNRGEC |
| Pepbody-SB1901-HL | GSPSGESSAPGSSSAESPGPGSESPSGSAPGESSPSGSAPGSPSGESSAPGS |
| heavy chain | PESGSASPGSSSAESPGPGSPSGESSAPGGGGSGGGGSMVPSALTASGG |
| (SEQ ID NO: 58) | GGGSGGGGSQVQLVQSGAEVKKPGASVKVSCKASGYTFTDDYIDWVR |
| | QATGQGLEWIGEIYPGSGNTYYNEKFKGRVTMTRDKSISTAYMELSSL |
| | RSEDTAVYYCARSQVYGKIAMDHWGQGTLVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCPP |
| | CPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGF |
| | YPSDIAVEWESNGQPENNYKTTPPVLDSDGSFFLYSKLTVDKSRWQQG |
| | NVFSCSVMHEALHNHYTQKSLSLSPGK |
| Pepbody-SB1901-HL | GSPSGESSAPGSSSAESPGPGSESPSGSAPGESSPSGSAPGSPSGESSAPGS |
| light chain | PESGSASPGSSSAESPGPGSPSGESSAPGGGGSGGGGSMVPSALTASGG |
| (SEQ ID NO: 61) | GGGSGGGGSDIVMTQSPDSLAVSLGERATINCRASESVDNSGNSFMHW |
| | YQQKPGQPPKLLIYRASNLESGVPDRFSGSGSRTDFTLTISSLQAEDVAV |
| | YYCQQSKEDPYTFGQGTKVEIKRTVAAPSVFIFPPSDEQLKSGTASVVC |
| | LLNNFYPREAKVQWKVDNALQSGNSQESVTEQDSKDSTYSLSSTLTLS |
| | KADYEKHKVYACEVTHQGLSSPVTKSFNRGEC |

### Example 12: Masked Antibody Prodrug Reduced Antibody Affinity and Function

### A. Effect of Masking Peptide on Antibody-Binding:

To evaluate the ability of the masking polypeptide in antibody prodrug to mask or inhibit binding of the anti-TNFR2 antibody, binding ELISA assay was performed by coating a plate with 50 μL/well human TNFR2 at 1 μg/mL at 4° C. overnight followed by adding 200 μL of blocking buffer (PBS containing 1% BSA) for 60 min at room temperature. After plate wash, 50 μL each of a serial diluted Pepbody-SB1901-H, Pepbody-SB1901-L, and Pepbody-SB1901-HL or SB1901-72 (an anti-TNFR2 antibody made in house) were loaded to wells and incubated at 37° C. for 120 minutes. After plate wash, Anti-mus FC-AP conjugated antibody at 1:2000 dilution in PBS containing 1% BSA was added to each well and incubated at 37° C. for 90 minutes. After plate wash, the plate was added with 50 μL of the freshly prepared substrate (pNPP in Tris buffer) to each well. The absorbance at 405 nm was obtained in a microplate reader (BioTec, USA).

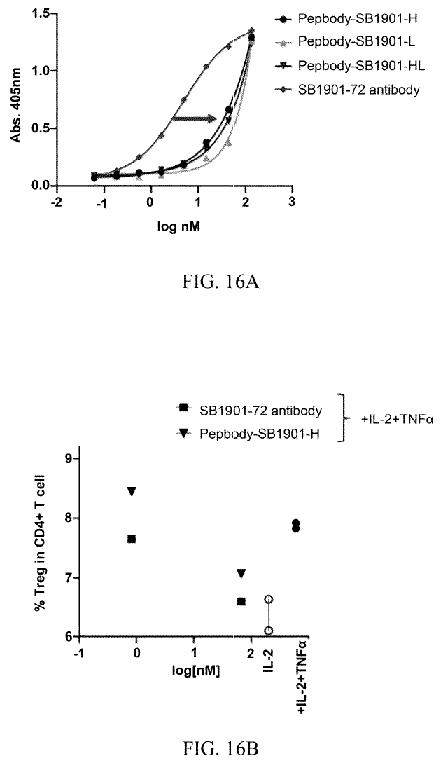

As shown in FIG. 16A, the prodrug Pepbody-SB1901-H, Pepbody-SB1901-L and Pepbody-SB1901-2-HL all had significantly reduced the binding to TNFR2 with comparison to the SB1901-72 antibody. This demonstrated that the masking polypeptide could also block the antibody SB1901-72 binding to its TNFR2 target, which suggested that the masking polypeptide could be used in antibody modality and prevented or reduced the binding of the antibody and its target.

### B. Effect of Masking Polypeptide on the Function of TNFR2 Antibody (SB1901-72) in Human Primary Treg Cell Proliferation Assay:

TNFR2 plays a critical role in human primary Treg cell proliferation. Blocking the TNFR2 signaling pathway using an anti-TNFR2 antibody can negate TNFα-induced Treg cell proliferation. This assay was exploited to test whether the masking peptide was able to cloak the effect of the anti-TNFR2 antibody SB1901-72.

Human PBMCs were incubated with 200U/mL IL-2 (Peprotech, cat #200-02) and 20 ng/ml TNFα (Peprotech, cat #300-01A) in the presence or absence of SB1901-72 or Pepbody-SB190-H in complete RPMI media in 96 well plates at 37° C. for 72 hours. Cells were stained with fluorescent-conjugated anti-CD3 antibody (BD Biosciences, cat #557705) and anti-CD4 antibody (Biolegend, cat #317424) in FACS buffer, followed by fix/permeabilization and intracellular staining with fluorescent-conjugated anti-Foxp3 antibody (Fisher Scientific, cat #50-151-75). Cells were fixed with 2% PFA and analyzed by flow cytometry with Attune. The percentage of Foxp3+ cells in CD4+ cells was analyzed with FlowJo software.

As shown in FIG. 16B, the Pepbody-SB1901-H had reduced the anti-TNFR2 antibody SB1901-72 inhibition of Treg cell proliferation to a certain degree. This data further ratified the masking polypeptide that could be used in antibody modality as well to block or reduce antibody function.

1. An IL-15 prodrug comprising: (i) one or more IL-15 cytokine (I), (ii) one or more cleavable moiety (CM), and (iii) one or more masking polypeptide (MP), wherein the cleavable moiety (CM) comprises the amino acid sequence of any one of SEQ ID NOs: 8-16, or a variant thereof having at least about 90% sequence identity to the amino acid sequence of any one of SEQ ID NOs: 8-16, the masking polypeptide (MP) attenuates the activity of IL-15 cytokine (I), and the cleavable moiety (CM) is susceptible to cleave at or near a tumor or a target cell.

2. (canceled)

3. The IL-15 prodrug according to claim 1, wherein the IL-15 cytokine (I) and the masking polypeptide (MP) are linked through the cleavable moiety (CM).

4. The IL-15 prodrug according to claim 1, wherein the IL-15 prodrug further comprises an IL-15Rα or a functional fragment thereof (S), and wherein:
(i) the IL-15Rα or a functional fragment thereof is selected from an extracellular domain of IL-15Rα or a sushi domain or functional analogs, or
(ii) the IL-15Rα or a functional fragment thereof (S) and masking polypeptide (MP) are linked through the cleavable moiety (CM), or
(iii) the IL-15Rα or a functional fragment thereof (S) and the IL-15 cytokine (I) are covalently linked; or the IL-15Rα or a functional fragment thereof (S) and the IL-15 cytokine (I) are non-covalently linked, and form an IL-15/IL-15Rα complex.

5. (canceled)

6. The IL-15 prodrug according to claim 1, wherein the IL-15 prodrug further comprises one or more half-life extension moiety (C).

7. The IL-15 prodrug according to claim 6, wherein:
(i) the IL-15 cytokine (I) is linked to the half-life extension moiety (C); and/or
(ii) the IL-15Rα or a functional fragment thereof (S) is linked to the half-life extension moiety (C); and/or
(iii) the masking polypeptide (MP) is linked to the half-life extension moiety (C) through the cleavable moiety (CM).

8. The IL-15 prodrug according to claim 7, wherein:
(i) the IL-15 cytokine (I) is linked to the half-life extension moiety (C), the IL-15Rα or a functional fragment thereof (S) is linked to the IL-15 cytokine (I), and the masking polypeptide (MP) is linked to the IL-15Rα or a functional fragment thereof (S) through the cleavable moiety (CM); or
(ii) the IL-15 cytokine (I) is linked to the half-life extension moiety (C), the IL-15Rα or a functional fragment thereof (S) is linked to the half-life extension moiety (C), and the masking polypeptide (MP) is linked to the IL-15 cytokine (I) through the cleavable moiety (CM); or
(iii) the IL-15Rα or a functional fragment thereof (S) is linked to the half-life extension moiety (C), the IL-15 cytokine (I) is linked to the IL-15R a or a functional fragment thereof (S), and the masking polypeptide (MP) is linked to the IL-15 cytokine (I) through the cleavable moiety (CM); or
(iv) the IL-15 cytokine (I) is linked to the half-life extension moiety (C), the IL-15Rα or a functional fragment thereof (S) is linked to the half-life extension moiety (C), and the masking polypeptide (MP) is linked to the IL-15Rα or a functional fragment thereof (S) through the cleavable moiety (CM); or
(v) the masking polypeptide (MP) is linked to the half-life extension moiety (C) through the cleavable moiety (CM), the IL-15Rα or a functional fragment thereof (S) is linked to the half-life extension moiety (C), and the IL-15 cytokine (I) is linked to the IL-15Rα or a functional fragment thereof (S); or
(vi) the masking polypeptide (MP) is linked to the half-life extension moiety (C) through the cleavable moiety (CM), the IL-15 cytokine (I) is linked to the half-life extension moiety (C), and the IL-15Rα or a functional fragment thereof (S) is linked to the IL-15 cytokine (I).

9. (canceled)

10. The IL-15 prodrug according to claim 1, wherein the prodrug is a dimer and comprises two monomers, and wherein:
(i) one monomer comprises the first half-life extension moiety (C), the IL-15Rα or a functional fragment thereof (S), the IL-15 cytokine (I); and the other monomer comprises the second half-life extension moiety (C), the masking polypeptide (MP) and the cleavable moiety (CM), wherein the masking polypeptide (MP) is linked to the second half-life extension moiety (C) through the cleavable moiety (CM); or
(ii) one monomer comprises the first half-life extension moiety (C), the IL-15Rα or a functional fragment thereof (S), the masking polypeptide (MP) and the cleavable moiety (CM); and the other monomer comprises the second half-life extension moiety (C) and the IL-15 cytokine (I); or
(iii) one monomer comprises the first half-life extension moiety (C), the IL-15 cytokine (I), the masking polypeptide (MP) and a cleavable moiety (CM); and the other monomer comprises the second half-life extension moiety (C) and the IL-15Rα or a functional fragment thereof (S); or
(iv) one monomer and the other monomer each comprises the half-life extension moiety (C), the IL-15 cytokine (I), the IL-15Rα or a functional fragment thereof (S), the cleavable moiety (CM) and the masking polypeptide (MP).

11. The IL-15 prodrug according to claim 10, wherein:
(i) in one monomer: both the IL-15 cytokine (I) and the IL-15Rα or a functional fragment thereof (S) are linked to the first half-life extension moiety (C); and in the other monomer: the masking polypeptide (MP) is linked to the second half-life extension moiety (C) through the cleavable moiety (CM); or
(ii) in one monomer: the IL-15 cytokine (I) is linked to the first half-life extension moiety (C) and the IL-15Rα or a functional fragment thereof (S) is linked to the IL-15 cytokine (I); and in the other monomer: the masking polypeptide (MP) is linked to the second half-life extension moiety (C) through the cleavable moiety (CM); or
(iii) in one monomer: the IL-15Rα or a functional fragment thereof (S) is linked to the first half-life extension moiety (C) and the IL-15 cytokine (I) is linked to the IL-15Rα or a functional fragment thereof (S); and in the other monomer: the masking polypeptide (MP) is linked to the second half-life extension moiety (C) through the cleavable moiety (CM); or
(iv) in one monomer: the IL-15Rα or a functional fragment thereof (S) is linked to the first half-life extension moiety (C) and the masking polypeptide (MP) is linked to the first half-life extension moiety (C) through the cleavable moiety (CM); and in the other monomer: the IL-15 cytokine (I) is linked to the second half-life extension moiety (C); or
(v) in one monomer: the IL-15Rα or a functional fragment thereof (S) is linked to the first half-life extension moiety (C) and the masking polypeptide (MP) is linked to the IL-15Rα or a functional fragment thereof (S) through the cleavable moiety (CM); and in the other monomer: the IL-15 cytokine (I) is linked to the second half-life extension moiety (C); or
(vi) in one monomer: the IL-15 cytokine (I) is linked to the first half-life extension moiety (C) and the masking polypeptide (MP) is linked to the first half-life extension moiety (C) through the cleavable moiety (CM); and in the other monomer: the IL-15Rα or a functional fragment thereof (S) is linked to the second half-life extension moiety (C); or
(vii) in one monomer: the IL-15 cytokine (I) is linked to the first half-life extension moiety (C) and the masking polypeptide (MP) is linked to the IL-15 cytokine (I) through the cleavable moiety (CM); and in the other monomer: the IL-15Rα or a functional fragment thereof (S) is linked to the second half-life extension moiety (C); or
(viii) one monomer and the other monomer each comprises the construct of any one of claim 8.

12. (canceled)

13. The IL-15 prodrug according to claim 1, wherein;
(i) the IL-15 cytokine (I) comprises one or more amino acids mutations selected from the group consisting of L45D, L45E, Q48K, S51D, L52D, E64K, I67D, 167E, I68D, and N72D; or
(ii) the IL-15 cytokine (I) comprises the amino acid sequence of any one of SEQ ID NOs: 22-23 and 67-76, or a variant thereof having at least about 90% sequence identity to the amino acid sequence of any one of SEQ ID NOs: 22-23 and 67-76.

14. (canceled)

15. The IL-15 prodrug according to claim 4, wherein the IL-15Rα or a functional fragment thereof (S) comprises the amino acid sequence of any one of SEQ ID NOs: 24-26, or a variant thereof having at least about 90% sequence identity to the amino acid sequence of any one of SEQ ID NOs: 24-26.

16. The IL-15 prodrug according to claim 6, wherein the half-life extension moiety (C) comprises an Fc domain; wherein:
(i) the Fc domain is selected from the group consisting of a human IgG1 Fc domain, a human IgG2 Fc domain, a human IgG3 Fc domain, a human IgG4 Fc domain, an IgA Fc domain, an IgD Fc domain, an IgE Fc domain, and an IgM Fc domain; or
(ii) the Fc domain is a human IgG1 Fc domain having L234A and L235A mutations, according to EU Numbering system; and/or
(iii) the Fc domains comprise knobs-into-holes mutations (Fc knob and Fc hole), wherein the Fc knob comprises a T366W mutation in the Fc domain, and the Fc hole comprises T366S, L368A, and Y407V mutations in the Fc domain, according to EU Numbering system, optionally, the Fc knob further comprises S354C mutation, and the Fc hole further comprises Y349C mutation, according to EU Numbering system.

17-21. (canceled)

22. The IL-15 prodrug according to claim 1, wherein:
(i) the masking polypeptide (MP) is composed of five types of amino acids G, S, P, E, and A, and further wherein the percentage of amino acid residue G in the masking polypeptide is about 15%-30%, preferably about 20%; the percentage of amino acid residue S in the masking polypeptide is about 20%-40%, preferably about 40%; the percentage of amino acid residue P in the masking polypeptide is about 15%-40%, preferably about 20%; the percentage of amino acid residue E in the masking polypeptide is about 1%-20%, preferably about 10%; and the percentage of amino acid residue A in the masking polypeptide is about 5%-20%, preferably about 10%; and when the number of amino acids is not an integer, take the integer value; or
(ii) the masking polypeptide (MP) is composed of four types of amino acids S, P, E, and G, and further wherein the percentage of amino acid residue S in the masking polypeptide is about 20%-40%, preferably about 23%; the percentage of amino acid residue P in the masking polypeptide is about 15%-40%, preferably about 29%, the percentage of amino acid residue E in the masking polypeptide is about 1%-20%, preferably about 18%, and the percentage of amino acid residue G in the masking polypeptide is about 15%-30%, preferably about 30%; and when the number of amino acids is not an integer, take the integer value; or
(iii) the masking polypeptide (MP) comprises about 40 to 720 amino acid residues; or
(iv) the masking polypeptide (MP) comprises about 80 to 320 amino acid residues; or
(v) the masking polypeptide (MP) comprises about 80 to 240 amino acid residues.

23-24. (canceled)

25. The IL-15 prodrug according to claim 1, wherein;
(i) the masking polypeptide (MP) comprises the amino acid sequence of SEQ ID NO: 6; or
(ii) the masking polypeptide (MP) comprises the amino acid sequence of SEQ ID NO: 1; or
(iii) the masking polypeptide (MP) comprises the amino acid sequence of any one of SEQ ID NOs: 1-5, or a variant thereof having at least about 90% sequence identity to the amino acid sequence of any one of SEQ ID NOs: 1-5.

26-30. (canceled)

31. The IL-15 prodrug according to claim 1, wherein the prodrug comprises two monomers, and wherein in one monomer, the IL-15Rα or a functional fragment thereof (S) is linked to the first Fc domain, and in the other monomer, the IL-15 cytokine (I) is linked to the second Fc domain, and the masking polypeptide (MP) is linked to the IL-15 cytokine (I) through the cleavable moiety (CM).

32. The IL-15 prodrug according to claim 31, wherein:
(1) one monomer comprises the amino acid sequence of SEQ ID NO: 33 or a variant thereof having at least about 80% sequence identity to the amino acid sequence of SEQ ID NO: 33, and the other monomer comprises the amino acid sequence of SEQ ID NO: 32 or a variant thereof having at least about 80% sequence identity to the amino acid sequence SEQ ID NO: 32; or
(2) one monomer comprises the amino acid sequence of SEQ ID NO: 34 or a variant thereof having at least about 80% sequence identity to the amino acid sequence of SEQ ID NO: 34, and the other monomer comprises the amino acid sequence of SEQ ID NO: 32 or a variant thereof having at least about 80% sequence identity to the amino acid sequence SEQ ID NO: 32; or
(3) one monomer comprises the amino acid sequence of SEQ ID NO: 36 or a variant thereof having at least about 80% sequence identity to the amino acid sequence of SEQ ID NO: 36, and the other monomer comprises the amino acid sequence of SEQ ID NO: 32 or a variant thereof having at least about 80% sequence identity to the amino acid sequence SEQ ID NO: 32; or
(4) one monomer comprises the amino acid sequence of SEQ ID NO: 37 or a variant thereof having at least about 80% sequence identity to the amino acid sequence of SEQ ID NO: 37, and the other monomer comprises the amino acid sequence of SEQ ID NO: 32 or a variant thereof having at least about 80% sequence identity to the amino acid sequence SEQ ID NO: 32; or
(5) one monomer comprises the amino acid sequence of SEQ ID NO: 38 or a variant thereof having at least about 80% sequence identity to the amino acid sequence of SEQ ID NO: 38, and the other monomer comprises the amino acid sequence of SEQ ID NO: 32 or a variant thereof having at least about 80% sequence identity to the amino acid sequence SEQ ID NO: 32; or
(6) one monomer comprises the amino acid sequence of SEQ ID NO: 39 or a variant thereof having at least about 80% sequence identity to the amino acid sequence of SEQ ID NO: 39, and the other monomer comprises the amino acid sequence of SEQ ID NO: 32 or a variant thereof having at least about 80% sequence identity to the amino acid sequence SEQ ID NO: 32; or
(7) one monomer comprises the amino acid sequence of SEQ ID NO: 45 or a variant thereof having at least about 80% sequence identity to the amino acid sequence of SEQ ID NO: 45, and the other monomer comprises the amino acid sequence of SEQ ID NO: 46 or a variant thereof having at least about 80% sequence identity to the amino acid sequence SEQ ID NO: 46; or
(8) one monomer comprises the amino acid sequence of SEQ ID NO: 47 or a variant thereof having at least about 80% sequence identity to the amino acid sequence of SEQ ID NO: 47, and the other monomer comprises the amino acid sequence of SEQ ID NO: 40 or a variant thereof having at least about 80% sequence identity to the amino acid sequence SEQ ID NO: 40; or
(9) one monomer comprises the amino acid sequence of SEQ ID NO: 33 or a variant thereof having at least about 80% sequence identity to the amino acid sequence of SEQ ID NO: 33, and the other monomer comprises the amino acid sequence of SEQ ID NO: 40 or a variant thereof having at least about 80% sequence identity to the amino acid sequence SEQ ID NO: 40; or
(10) one monomer comprises the amino acid sequence of SEQ ID NO: 62 or a variant thereof having at least about 80% sequence identity to the amino acid sequence of SEQ ID NO: 62, and the other monomer comprises the amino acid sequence of SEQ ID NO: 63 or a variant thereof having at least about 80% sequence identity to the amino acid sequence SEQ ID NO: 63; or
(11) one monomer comprises the amino acid sequence of SEQ ID NO: 41 or a variant thereof having at least about 80% sequence identity to the amino acid sequence of SEQ ID NO: 41, and the other monomer comprises the amino acid sequence of SEQ ID NO: 63 or a variant thereof having at least about 80% sequence identity to the amino acid sequence SEQ ID NO: 63; or
(12) one monomer comprises the amino acid sequence of SEQ ID NO: 64 or a variant thereof having at least about 80% sequence identity to the amino acid sequence of SEQ ID NO: 64, and the other monomer comprises the amino acid sequence of SEQ ID NO: 65 or a variant thereof having at least about 80% sequence identity to the amino acid sequence SEQ ID NO: 65; or
(13) one monomer comprises the amino acid sequence of SEQ ID NO: 62 or a variant thereof having at least about 80% sequence identity to the amino acid sequence of SEQ ID NO: 62, and the other monomer comprises the amino acid sequence of SEQ ID NO: 65 or a variant thereof having at least about 80% sequence identity to the amino acid sequence SEQ ID NO: 65; or
(14) one monomer comprises the amino acid sequence of SEQ ID NO: 41 or a variant thereof having at least about 80% sequence identity to the amino acid sequence of SEQ ID NO: 41, and the other monomer comprises the amino acid sequence of SEQ ID NO: 65 or a variant thereof having at least about 80% sequence identity to the amino acid sequence SEQ ID NO: 65; or
(15) one monomer comprises the amino acid sequence of SEQ ID NO: 66 or a variant thereof having at least about 80% sequence identity to the amino acid sequence of SEQ ID NO: 66, and the other monomer comprises the amino acid sequence of SEQ ID NO: 65 or a variant thereof having at least about 80% sequence identity to the amino acid sequence SEQ ID NO: 65; or
(16) one monomer comprises the amino acid sequence of SEQ ID NO: 43 or a variant thereof having at least about 80% sequence identity to the amino acid sequence of SEQ ID NO: 43, and the other monomer comprises the amino acid sequence of SEQ ID NO: 46 or a variant thereof having at least about 80% sequence identity to the amino acid sequence SEQ ID NO: 46; or
(17) one monomer comprises the amino acid sequence of SEQ ID NO: 39 or a variant thereof having at least about 80% sequence identity to the amino acid sequence of SEQ ID NO: 39, and the other monomer comprises the amino acid sequence of SEQ ID NO: 40 or a variant thereof having at least about 80% sequence identity to the amino acid sequence SEQ ID NO: 40; or
(18) one monomer comprises the amino acid sequence of SEQ ID NO: 41 or a variant thereof having at least about 80% sequence identity to the amino acid sequence of SEQ ID NO: 41, and the other monomer comprises the amino acid sequence of SEQ ID NO: 87 or a variant thereof having at least about 80% sequence identity to the amino acid sequence SEQ ID NO: 87; or
(19) one monomer comprises the amino acid sequence of SEQ ID NO: 41 or a variant thereof having at least about 80% sequence identity to the amino acid sequence of SEQ ID NO: 41, and the other monomer comprises the amino acid sequence of SEQ ID NO: 91 or a variant thereof having at least about 80% sequence identity to the amino acid sequence SEQ ID NO: 91; or
(20) one monomer comprises the amino acid sequence of SEQ ID NO: 41 or a variant thereof having at least about 80% sequence identity to the amino acid sequence of SEQ ID NO: 41, and the other monomer comprises the amino acid sequence of SEQ ID NO: 93 or a variant thereof having at least about 80% sequence identity to the amino acid sequence SEQ ID NO: 93; or
(21) one monomer comprises the amino acid sequence of SEQ ID NO: 41 or a variant thereof having at least about 80% sequence identity to the amino acid sequence of SEQ ID NO: 41, and the other monomer comprises the amino acid sequence of SEQ ID NO: 94 or a variant thereof having at least about 80% sequence identity to the amino acid sequence SEQ ID NO: 94.

33-34. (canceled)

35. An isolated nucleic acid molecule that encodes the IL-15 prodrug of claim 1.

36. A vector comprising the isolated nucleic acid molecule of claim 35.

37. An isolated host cell comprising the vector of claim 36.

38. (canceled)

39. A pharmaceutical composition comprising the IL-15 prodrug of claim 1, and a pharmaceutically acceptable carrier moiety or excipients.

40. A method of treating a disease or condition in an individual in need thereof, comprising administering to the individual an effective amount of the IL-15 prodrug of claim 1, wherein the disease or condition is a cancer, an infectious disease or stimulating the immune system in a patient in need thereof, optionally, the cancer is selected from the group consisting of prostate cancer, colon cancer, renal carcinoma, melanoma, lung cancer, breast cancer, thyroid cancer, bladder cancer, gastric and esophageal cancer, pancreatic cancer, liver cancer, brain cancer, head and neck cancer, neuroblastoma, soft tissue carcinoma, lymphoma, leukemia, multiple myeloma, or any metastases therefrom.

41-42. (canceled)
